(19) United States
(12) Patent Application Publication
(10) Pub. No.: US 2023/0053013 A1
(43) Pub. Date: Feb. 16, 2023

(54) COMPOSITIONS AND METHODS FOR OPTOGENETIC IMMUNOTHERAPY

(71) Applicant: University of Massachusetts, Boston, MA (US)

(72) Inventors: Gang Han, Shrewsbury, MA (US); Yubin Zhou, Bellaire, TX (US)

(21) Appl. No.: 17/777,363

(22) PCT Filed: Nov. 24, 2020
(86) PCT No.: PCT/US20/61944

§ 371 Date: May 17, 2022

Related U.S. Application Data
(60) Provisional application No. 62/942,770, filed on Dec. 3, 2019.

(51) Int. Cl. C07K 14/725; C07K 14/705; C07K 16/28

(52) U.S. Cl. CPC C07K 14/7051; C07K 14/70517; C07K 16/2803; C07K 14/70578; C07K 2319/03; C07K 2319/02; C07K 2319/70; C07K 2317/622

(57) ABSTRACT

The invention provides novel light-switchable CAR T-cells that can be remotely controlled through NIR-light-converting upconvension nanoparticles, and related CAR T constructs, nanoparticles, compositions and methods thereof for optogenetic therapy.

### PRIORITY CLAIMS AND RELATED APPLICATIONS

This application claims the benefit of priority to U.S. Provisional Application No. 62/942,770, filed Dec. 3, 2019, the entire content of which is incorporated herein by reference for all purposes.

### STATEMENT REGARDING FEDERALLY SPONSORED RESEARCH OR DEVELOPMENT

This invention was made with government support under Grant no. CA232017, awarded by the National Institutes of Health. The Government has certain rights in the invention.

### TECHNICAL FIELDS OF THE INVENTION

The invention generally relates to immunotherapies and therapeutic methods. More particularly, the invention provides novel light-switchable chimeric antigen receptor T-cells that can be remotely controlled through MR-light-converting upconvension nanoparticles, and related photoswitchable chimeric antigen receptor constructs and upconvension nanoparticles, and pharmaceutical compositions and methods thereof for optogenetic therapy.

### BACKGROUND OF THE INVENTION

Cancer is a group of diseases involving abnormal cell growth with the potential to invade or spread to other parts of the body. As one of the deadliest threats to human health, more than 90 million people are believed to have cancer globally. In the U.S. alone, cancer affects nearly 1.3 million new patients each year, and is the second leading cause of death after cardiovascular disease, accounting for approximately 1 in 4 deaths. The most common types of cancer in males are lung cancer, prostate cancer, colorectal cancer and stomach cancer. In females, the most common types are breast cancer, colorectal cancer, lung cancer and cervical cancer. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult.

Chimeric antigen receptor (CAR) T-cell immunotherapy has demonstrated high potential for the elimination of tumors, particularly in patients with CD19-positive lymphoma and leukemia. (June, et al. 2018 Science 359, 1361-1365.) CAR-T cell therapy uses T cells engineered with CARs for cancer therapy. CARs (a.k.a., chimeric immunoreceptors, chimeric T cell receptors or artificial T cell receptors) are synthetic receptors engineered onto the surface of T cells, where they can engage specific tumor antigens in a major histocompatibility complex (MHC)-independent manner. (Chmielewski, et al. 2013 Front Immunol 4, 371.) Antigen recognition allows engineered T cells to be activated and subsequently perform their killing/effector activities toward tumor cells.

A typical FDA-approved second-generation CAR contains an antibody-derived single chain variable fragment (scFv) in the extracellular domain, a transmembrane domain, costimulatory signals (e.g., 4-1BB or CD28), and the intracellular signal transduction component known as the immunoreceptor tyrosine-based activation motifs (ITAMs) derived from CD3. The latter component constitutes an essential subunit of the T cell receptor required for T cell activation. (Sadelain, et al. 2013 Cancer Discov 3, 388-398.) Despite of the tremendous success of CAR T-cell therapy in cancer treatment, this type of immunotherapy imposes significant safety challenges due to the lack of control over the dose, location, and timing of T cell activity. (June, et al. 2018 Science 359, 1361-1365; Sadelain, et al. 2013 Cancer Discov 3, 388-398; Grupp, et al. 2013 N Engl J Med 368, 1509-1518; Maude, et al. 2018 N Engl J Med 378, 439-448.) This is most notably exemplified by the cytokine release syndrome (CRS) and the “on-target, off-tumor” cytotoxicity.

While the recently FDA approved CAR T-cell therapies (Kymriah and Yescarta) are designed to target CD19-positive tumors, they cannot discriminate between normal CD19⁺ cells and cancerous CD19⁺ cells. (Maude, et al. 2018 N Engl J Med 378, 439-448; Neelapu, et al. 2017 N Engl J Med 377, 2531-2544.) As such, CAR T-cells will likely attack normal cells or tissues, leading to B cell aplasia or even more devastating systemic consequences in certain patients, thereby posing limitations on the use of the current CAR T-cell therapy. (Kochenderfer, et al. 2012 Blood 119, 2709-2720; Magee, et al. 2014 Discov Med 18, 265-271.) Another prevalent side effect association with CAR T-cell therapy is CRS, which arises from uncontrolled release of inflammatory cytokines, including interferon-gamma, IL-6, IL-10 and granulocyte macrophage colony-stimulating factor. Depending on the degree of CRS severity, patients receiving CAR T-cell therapy may manifest one or more of the following clinical symptoms: high fever, malaise, fatigue, myalgia, nausea, anorexia, tachycardia/hypotension, capillary leak, cardiac dysfunction, renal impairment, hepatic failure, and disseminated intravascular coagulation.

Aside from hematological malignancies, encouraging progress has been made in the development of CAR T-cell therapies against solid tumors, such as those targeting the carcinoembryonic antigen in metastatic gastrointestinal malignancies, the EGFRvIII antigen in glioblastoma, and the ganglioside antigen in neuroblastoma. (Thistlethwaite, et al. 2017 Cancer Immunol Immunother 66, 1425-1436; Han, et al. 2015 Sci Rep 5, 11483; O'Rourke, et al. 2017 Sci Trans' Med 9; Prapa, et al. 2015 Oncotarget 6, 24884-24894; Rossig, et al. 2001 Int J Cancer 94, 228-236, doi:10.1002/ijc.1457; Richards, et al. 2018 Front Immunol 9, 2380; Newick, et al. 2017 Annu Rev Med 68, 139-152.)

CAR T-cell therapies for solid tumor, however, are far less successful because of three major barriers: the hostile tumor microenvironment, limited CAR T-cell trafficking and infiltration into tumor sites, and lack of strict specificity toward tumors. To overcome these limitations, several strategies have been proposed, including arming CAR-T cells with knockout of PD-1 expression to eliminate the suppressive tumor microenvironment, modulating chemokine receptors expression to promote trafficking and infiltration, as well as secretion of immunomodulatory cytokines by engineered CAR T-cells. Despite these efforts, CAR T-cell therapies for solid tumor are still under clinical trials and have not yet reached the milestone of FDA approval. (Thistlethwaite, et al. 2017 Cancer Immunol Immunother 66, 1425-1436; Han, et al. 2015 Sci Rep 5, 11483; Prapa, et al. 2015 Oncotarget 6, 24884-24894; Scarfo, et al. 2017 J Immunother Cancer 5, 28; Pule, et al. 2008 Nat Med 14, 1264-1270; D′Aloia, et al. 2018 Cell Death Dis 9, 282; Craddock, et al. 2010 J Immunother 33, 780-788; Chmielewski, et al. 2011 Cancer Res 71, 5697-5706.)

Although CAR T cell-based immunotherapy has demonstrated promising curative potential for cancer treatment, it lacks precise control over the location and duration of anti-tumor immune response. Intelligent CAR-T cell-based therapies with precise spatiotemporal control over therapeutic activities are urgently needed.

### SUMMARY OF THE INVENTION

The invention provides a revolutionary approach based on light-switchable CAR T-cell (“LiCAR”) that enables photo-tunable activation of therapeutic T cells to induce tumor cell killing both in vitro and in vivo. When coupled with imaging guided surgically removable upconversion nanoparticles (e.g., nanoplates) that have enhanced near infrared (NIR)-to-blue upconversion luminescence as miniatured deep tissue transducers, LiCAR enables precise spatiotemporal control over CAR T cell-mediated anti-tumor therapeutic activity. This remotely controllable nano-optogenetic device sets the stage for the exploration of optogenetic immunotherapy to deliver personalized anti-cancer therapy.

For example, a proof-of-principle study is disclosed herein on the targeting of the CD19 antigen in order to validate an intelligent nano-optogenetic CAR-T cell therapy, which address the key and urgent challenges for the FDA-approved CAR-T cells and promise to overcome major safety challenges associated with the existing immunotherapies

In one aspect, the invention generally relates to an isolated nucleic acid sequence or isolated nucleic acid sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a non-covalent dimer upon light induction, Component I and Component II together form a functional CAR.

In another aspect, the invention generally relates to a vector that comprises a nucleic acid sequence or nucleic acid sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a non-covalent dimer upon light induction, Component I and Component II together form a CAR.

In yet another aspect, the invention generally relates to a cell comprising a nucleic acid sequence or sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a CAR.

In yet another aspect, the invention generally relates to a pharmaceutical composition comprising a cell population comprising an anti-tumor effective amount of a cell disclosed herein.

In yet another aspect, the invention generally relates to an upconversion nanoparticle (UCNP) having a core-shell structure, which comprises: a core comprising β-NaYbF₄:Tm; and a shell comprising β-NaYF₄.

In yet another aspect, the invention generally relates to a pharmaceutical composition comprising a UCNP disclosed herein.

In yet another aspect, the invention generally relates to a pharmaceutical composition comprising a herein disclosed UCNP and a disclosed herein cell.

In yet another aspect, the invention generally relates to a method for activating a light sensitive biological agent or cell. The method comprises: administering to a subject in need thereof a UCNP and a light sensitive biological agent or cell; exciting the UCNP with an excitation light to cause the UCNP to emit a luminescence at a wavelength capable of sensitizing the biological agent or cell; and activating the light sensitive biological agent or cell.

In yet another aspect, the invention generally relates to a method for treating cancer in a subject. The method comprises: administering to the subject in need thereof a pharmaceutical composition comprising an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for treating cancer in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for providing anti-tumor immunity in a subject. The method comprises: administering to the subject in need thereof a pharmaceutical composition comprising an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for providing anti-tumor immunity in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for stimulating a T cell-mediated immune response to a cell population or tissue in a subject. The method comprises: administering to the subject in need thereof a pharmaceutical composition comprising an anti-tumor effective amount of a population of T cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for stimulating a T cell-mediated immune response to a cell population or tissue in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for generating a persisting population of genetically engineered T cells in a subject. The method comprises: administering to the subject in need thereof a pharmaceutical composition comprising an anti-tumor effective amount of a population of T cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for generating a persisting population of genetically engineered T cells in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

### BRIEF DESCRIPTION OF THE DRAWINGS

FIG. 1. Design of LiCAR for light-inducible assembly of functional chimeric antigen receptors (CARs). a, Design of photoactivatable CARs that are dually gated by tumor antigen (CD19) and light. Engineered CAR T-cells can only be switched on in the presence of light when engaged with cognate tumor cells. b, Optimized constructs used in the study (see FIG. 6 for the full list). Modular domains within a typical CAR were split into two polypeptides (constructs A+B or C+D), the functional assembly of which can only be achieved upon light-induced interaction using two optical dimerizers (CRY2/CIBN or LOV2-ssrA/sspB [or iLID] pairs) with different binding properties. Weaker versions of iLID (mutations R73Q or A58V in sspB; constructs D4.1-4.4) were used to reduce the degrees of LiCAR T-cell pre-activation. T cell activation was assessed using two independent assays: (i) NFAT-dependent luciferase (NFAT-Luc) reporter gene expression; and (ii) IL-2 production upon incubation with cognate antigen-bearing tumor cells (CD19⁺ Raji or Daudi lymphoma cells). The degree of activation was indicated by the darkness of the box on the right. c-h, Representative confocal images showing reversible recruitment of cytosolic Construct B or D (mCherry-tagged; magenta) toward the PM-resident Construct A (GFP-tagged; green; c) or Construct C (d-e) in response to two dark-light cycles (40 mW/cm²; 470 nm) in Hela cells. (f-h) The activation and deactivation kinetics were shown below the images. n=4 (panel f); n=36 (panel g); n=11 (panel h; mean±s.e.m.). Scale bar, 5 um. i, Quantification of NFAT-dependent luciferase (NFAT-Luc) reporter activity in Jurkat T cells. CAR, LiCAR, or defective LiCAR-transduced T cells engaged tumor cells bearing noncognate (open box; human CD19-negative K562 cells) or cognate antigens (red box; hCD19⁺ Raji cells) under dark (open box) or lit conditions (blue box). Blue light (40 mW/cm² at 470 nm) was applied for 20 min and then in pulsed cycles of 30 sec ON+100 sec OFF for 8 h. Defective LiCAR lacking the CD3ζTAM domain was used as negative control. n=3 (mean±s.e.m.). ****P<0.0001 compared to the dark group; n.s., not significant (two-tailed Student's t-test).

FIG. 2. Photo-tunable immune response enabled by LiCAR T-cells. a, Human Jurkat T cells were virally transduced to express WT CAR, LiCARs, or defective LiCAR, followed by functional assays to determine their therapeutic responses. b-c, Light-tunable responses of engineered CAR T-cells examined by (b) photo-inducible NFAT-dependent luciferase reporter activity and (c) IL-2 production in Jurkat T cells. n=3 (mean±s.e.m.). ON time of constant photostimulation was indicated at the bottom. *P<0.05; **P<0.01; ***P<0.001; ****P<0.0001 compared to the dark group or the lit groups; n.s., not significant (two-tailed Student's t-test). d-e, Determination of early T cell activation as reflected by upregulated CD69 expression on cell surface. Jurkat T cells transduced with conventional CAR, defective LiCAR, and LiCAR were engaged with hCD19³⁰ Daudi or hCD19⁻K562 cells under dark or lit conditions. n=3 (mean±s.e.m.). ****P<0.0001 compared to dark group; n.s., not significant (two-tailed Student's t-test). f, Primary human CD8⁺ T cells isolated from the peripheral blood of healthy donors were transduced to express WT CAR, LiCAR, or defective LiCAR, followed by functional assays (SYTOX Blue dead cell staining) to determine the tumor cell killing efficacy. The tumor killing results, quantified by SYTOX staining, are shown in the bar graph below the cartoon. n=2 (mean±range). **P<0.01 compared to dark group; n.s., not significant (two-tailed Student's t-test). g-h, Light-induced tumor cell killing assessed by flow cytometry (g) and time-lapse confocal imaging (h). Transduced human CD8⁺ T cells were engaged with hCD19⁻ K562 or hCD19⁺ Daudi lymphoma cells and kept in the dark or exposed to blue light. SYTOX blue was used to stain dying tumor cells with compromised plasma membranes. Shown in panel h are overlaid images of LiCAR-expressing human CD8⁺ T cells (green, C-GFP; red, D4-mCherry) engaged with Daudi cells (indicated by numbers). Red arrowheads, dying Daudi cells with positive SYTOX blue staining. Cells were either kept in the dark or exposed to blue light illumination (470 nm; 40 mW/cm²; 5 h). Scale bar, 5 μm.

FIG. 3. A nano-optogenetic strategy for selective destruction of melanoma using LiCAR T-cells. a, Quantification of IFN-γ produced by engineered mouse CD8⁺ T cells expressing LiCAR or defective LiCAR after co-culturing with melanoma cells (B16-OVA cells expressing human CD19 [B16-OVA-hCD19] or B16-OVA [as control]) at different effector: target (E:T) ratios. n=3 (mean±s.e.m.). ****P<0.0001 compared to the dark group; n.s., not significant (two-tailed Student's t-test). b, B16-OVA-hCD19 cell killing efficacy of mouse CD8⁺ T cells expressing WT CAR, LiCAR, or defective LiCAR at the indicated effector: target (E:T) ratios. n=4 (mean±s.e.m.). *P<0.05; ***P<0.001 compared to the dark group; n.s., not significant (two-tailed Student's t-test). c, Schematic of the core/shell structure of silica-coated upconversion nanoplates (UCNPs; βNaYbF4: 0.5% Tm@NaYF₄). d, Size measurement of silica coated β-NaYbF4:0.5% Tm@NaYF4 core-shell UCNPs by dynamic light scattering. The inset shows a representative transmission electron micrograph (Scale bar, 100 nm). e, The upconversion luminescence spectrum of synthesized UCNPs upon MR light stimulation at 980 nm. f, Schematic of the in vivo experimental setup. 2×10⁶ mouse CD8⁺ T cells expressing LiCAR and 150 μg of UCNPs were adoptively transferred into each tumor site of C57BL/6J mice 9 days after melanoma inoculation (B16-OVA or B16-OVA-hCD19). Mice were subjected to pulsed MR light stimulation for 8-9 days (980 nm at a power density of 250 mW/cm²; pulse of 20 sec ON, 5 minutes OFF; 2 h/day). At day 18 or 19, mice were euthanized for tumor isolation and phenotypic analyses. g, LiCAR CD8⁺ T-cells selectively destruct CD19-expressing melanoma in response to MR light illumination. Left, C57BL/6J mice were intradermally inoculated with 2.5×10⁵ B16-OVA-hCD19 in the left flank (red circle) and 2.5×10⁵ B16-OVA cells (CD19-negative tumor as control; blue circle) in the right flank. Two representatives mice are shown after treatment with 2×10⁶ LiCAR T-cells+150 μg UCNPs and exposed to the previously described MR pulses for 9 days. Middle, tumor sizes at the indicated time points were measured by a digital caliper with the tumor areas calculated in mm² (length×width). ***P<0.001 when compared to the CD19-negative B16-OVA group (paired two-tailed Student's t-test; n=5; mean s.e.m.). Right, isolated B16-OVA and B16-OVA-hCD19 tumors at day 19. Also see FIG. 16a. h, LiCAR T-cells permit MR light-inducible killing of B16-OVA-hCD19 melanoma in selected regions. Left, C57BL/6J mice were intradermally inoculated at both flanks with 3×10⁵B16-OVA-hCD19 cells. After injection with the 2×10⁶ LiCAR T-cells+150 μg UCNP mixture, the left flank (red circle) was exposed to MR pulses for 8 days, while the right side (blue circle) was protected from MR light using aluminum foil. Two representative mice are shown at day 18. Middle, tumor sizes measured at the indicated time points. Right, isolated B16-OVA-hCD19tumors with and without exposure to MR light at day 18. *P<0.05 when compared to the non-MR group (paired two-tailed Student's t-test; n=5; mean±s.e.m.). Note that more melanoma cells were injected so that we could collect sufficient tumor masses (without clearance as seen in panel h) for FACS analysis. FIG. 16b.

FIG. 4. mLiCAR T-cells reduce “on-target off-tumor” effects in a syngeneic mouse model of melanoma. a, Design of constructs to recognize mouse CD19 (mCD19) antigen overexpressed in B16 murine melanoma cells. The mCD19-recognizing scFv in WT mCAR or mLiCAR was derived a mouse mAb (clone 1D3). b, The engagement of 1D3 scFv to mCD19 antigen was quantified by NFAT-dependent luciferase (NFAT-Luc) reporter activity in Jurkat T cells. Jurkat T cells transduced with human CD19 (hCD19)-recognizing (WT hCAR) or mouse mCD19 antigen-recognizing constructs (WT mCAR, mLiCAR, or defective mLiCAR) were engaged with the corresponding tumor cells bearing noncognate (open box; B16-OVA cells) or the cognate antigens (B16-OVA-hCD19 for hCAR or B16-OVA-mCD19 for mCAR, mLiCAR, or defective mLiCAR groups) under dark (open box) or lit conditions (blue box). Blue light (40 mW/cm² at 470 nm) was applied for 20 min and then in pulsed cycles of 30 sec ON+100 sec OFF for 8 h. n=4 (mean±s.e.m.). ****P<0.0001 compared to dark group; n.s., not significant (two-tailed Student's t-test). c-d, WT mCAR- (c) or mLiCAR-expressing (d) murine CD8⁺ T-cells selectively destroy mCD19-expressing melanoma cells in vivo. mLiCAR T-cells exhibit MR-light dependent killing of B16-OVA-mCD19 tumor cells. Left, C57BL/6J mice were intradermally inoculated with 2.5×10⁵ B16-OVA cells in the left flank (as control without mCD19 overexpression, blue circle) and 2.5×10⁵ B16-OVA-mCD19 cells (red circle) in the right flank. Mice were treated with WT mCAR T-cells+150 μg UCNPs for 10 days (panel c), or 2×10⁶ mLiCAR T-cells+150 μg UCNPs with subsequent exposure to MR pulses for 10 days (panel d). Middle, Quantification of the tumor sizes. Tumor sizes at the endpoint after UCNP removal were measured by a digital caliper with the tumor areas calculated in mm² (length x width). *P<0.05 when compared to the the B16-OVA control group at day 19 (paired two-tailed Student's t-test; n=4 for panel c and n=3 for panel d; mean±s.e.m.). Right, Representative images of isolated B16-OVA and B16-OVA-mCD19 tumors with UCNPs at day 19. e, On-target off-tumor effects of mWT CAR and mLiCAR T-cells evaluated by the degree of B cell aplasia. Peripheral blood B cells from the WT mCAR or mLiCAR T-cell treated groups (as in panels c-d) inoculated with tumor cells were counted and compared on day 0 and day 3. B cells from peripheral blood of healthy mice were used as control. **P<0.01 (paired two-tailed Student's t-test; n=4; mean±s.e.m.) f, Representative H&E staining images of major organs isolated from mLiCAR T-cells/UCNP/NIR treated mice bearing tumors or healthy mice subcutaneously administered with 100 μl PBS. Scale bar, 100 μm.

FIG. 5. LiCAR T-cells mitigate cytokine release syndrome (CRS). a, Schematic of the CRS experimental setup. Raji tumor cells (3×10⁶) were injected (i.p.) into SCID-beige mice. After tumor growth for 3 weeks, WT CAR T cells/UCNPs or LiCAR T/UCNPs cells that could engage hCD19-expressing Raji cells were subsequently implanted to the tumor cell-injection sites. LiCAR (combination of C+D4.1)-treated mice were subjected to pulsed MR light stimulation for 3 days (980 nm at a power density of 250 mW/cm²; pulses of 20 sec ON, 5 minutes OFF; 2 h/day). Weight change was monitored every day. On day 0 (pre-CAR) and day 3, blood/serum was collected from the retro-orbital sinus by glass capillary from anesthetized mice. b, Weight change of WT CAR T-cells/UCNPs or LiCAR T-cells/UCNPs/NIR-treated mice bearing Raji tumors. Weight of each mouse was normalized to the starting point before CAR T-cell implantation. **P<0.01 when compared to the WT CAR T-cells group at 72 h (paired two-tailed Student's t-test; n=3; mean±s.e.m.) c, ELISA measurements of the serum levels of mIL-6 at 72h after WT CAR T-cells/UCNPs or LiCAR T-cells/UCNPs/NIR treatment or before CAR T cell injection (Pre-CAR) into the SCID-beige mice. *P<0.05 when compared to WT CAR T-cells group (paired two-tailed Student's t-test; n=3; mean±s.e.m.)

FIG. 6. Design and screening of CRY2- and LOV2-based LiCARs. (related to FIG. 1) a, Constructs used to screen and optimize the designed LiCARs (Components I+II). The inset showed confocal images of HeLa cells expressing the PM-embedded constructs A or C (without fluorescent tag) after non-permeabilized immunostaining with an anti-Myc antibody. For Component II expressed as a cytosolic protein, we also monitored light-inducible cytosol-to-PM translocation to confirm the photo-responsiveness of the optical dimerizer. b-d, Representative confocal images of HeLa cells expressing the indicated components before and after light illumination. The introduction of ER trafficking/export signals in Component I significantly improved PM targeting; whereas the addition of NES to Component II substantially reduced nuclear accumulation (b/c vs d). e-f, Representative confocal images of HeLa cells co-expressing constructs A+B3 or C+D4. Note that B3 and D4 contained the CD8 transmembrane domain (TM) and were thus embedded in the plasma membrane. Scale bar, 5 μm.

FIG. 7. Expression of engineered CAR components (A+B or C+D combinations) in human Jurkat T cells. (related to FIG. 1) a, Quantification of transduction efficiency in Jurkat T cells. Jurkat cells were retrovirally transduced with the indicated A+B or C+D combinations to assemble functional LiCARs. The expression of A/C (GFP⁺) or B/D (mCherry⁺) components was determined by flow cytometry. b, Immunoblot analysis of LiCAR components expression in Jurkat T cells. T cells were transduced with retroviruses encoding GFP-tagged WT CAR or LiCAR components (A-GFP+B-mCherry; or C-GFP+D-mCherry). Component I was probed with an anti-GFP antibody whereas Component II was probed with an anti-mCh antibody. GAPDH was used as a loading control.

FIG. 8. Optimizing the ratio of effector T cells to target tumor cells (E/T ratio) to evaluate the function of engineered CAR T-cells. (related to FIG. 2) a, Quantification of CD19 expression in target B cells. K562 cells showed negligible human CD19 (hCD19) expression whereas Raji or Daudi lymphoma cells showed over 98% positive staining for hCD19. b, NFAT-Luc activity of conventional CAR T-cells when co-cultured with different amounts of either hCD19⁻ K562 cells (open box) or hCD19⁺ Raji cells (red box). An E/T ratio of 1:3 was able to elicit the highest NFAT-dependent activity. n=3 (mean±s.e.m.). c, NFAT-Luc activity of WT CAR T-cells incubated with Raji cells at a ratio of 1:3 compared with that of non-transduced (negative control) T cells. n=3 (mean±s.e.m.).

FIG. 9. Early T cell activation reported by cell surface expression of CD69. (related to FIG. 2) a, Quantification of CD69 expression levels in Jurkat T cells expressing WT CAR, defective LiCAR (C+D5 lacking the CD3 subunit), or LiCAR (C+D4) before (open box) and after light illumination (blue box; 20 min and then 10 h with the pulse of 10 s ON+60 s OFF) after co-incubation with hCD19-negative K562 cells (open box) or hCD19-positive Raji cells (red box). b, Quantification of CD69 expression in Jurkat T cells co-cultured with hCD19-negative K562 (open box) or hCD19-positive Raji cells (red box). n=2 (mean±range).

FIG. 10. Expression of engineered CARs in human primary CD8⁺ T cells. (related to FIG. 2) a, Evaluation of the purity of CD8⁺ T cells isolated from PBMCs of healthy donors. Isolated T cells were stained with anti-CD8-APC. Non-stained CD8⁺ T cells were used as negative control to aid the gating of cell populations. b, Quantification of WT CAR (GFP-tagged), LiCAR (C-GFP+D4-mCh) or defective LiCAR (C-GFP+D5-mCh) expression in human CD8⁺ T cells. GFP-positive (for the WT CAR group) or double positive cells (for the LiCAR and the defective LiCAR groups) were used for functional assays. c, Representative confocal images of human CD8⁺ T-cells transduced with WT CAR (green, top panel) or the indicated CAR components (C-GFP, green; or D4/D5-mCherry, red; middle and bottom panels). d, Time-lapse imaging of tumor cells (Daudi) co-cultured with human CD8⁺ T cells (asterisk) expressing either WT CAR (GFP-tagged, top) in the dark or defective LiCAR (C-GFP+D5-mCh, bottom) exposed to blue light. Dying cells were made visible by SYTOX blue staining. WT CAR showed potent tumor killing ability in the absence of light; whereas defective CAR T-cells did not trigger tumor killing even under photostimulation.

FIG. 11. Expression of engineered CARs in mouse primary CD8⁺ T cells. (related to FIG. 3) a, Quantification of CD19 expression in melanoma cells (B16-OVA) and melanoma cells exogenously expressing human CD19 (B16-OVA-hCD19). b, Evaluation of the purity of CD8⁺ T cells isolated from mouse spleens. Isolated T cells were stained with an anti-mouse CD8a eFluor 450 antibody. Non-stained CD8⁺ T cells were used as negative control to aid the gating. c, Quantification of WT CAR (GFP-tagged), LiCAR (C-GFP+D4-mCh) or defective LiCAR (C-GFP+D5-mCh) expression in transduced mouse CD8⁺ T cells. GFP-positive (for the WT CAR group) or double positive cells (for the LiCAR and the defective LiCAR groups) were used for functional assays. d, Assessing the viability of murine melanoma cells expressing hCD19 (B16-OVA-hCD19) after co-culture with engineered mouse CD8⁺ T-cells. Mouse CD8 T cells expressing WT CAR, LiCAR or defective LiCAR were co-cultured with 1,000 pre-seeded B16-OVA-hCD19 cells at the indicated E:T ratios and incubated in the dark or exposed to blue light. Floating dead cells and CAR T-cells were washed away and viable tumor cells, which remained attached to the plate bottom, were made visible by DAPI staining under a confocal microscope. LiCAR T-cells effectively killed tumor cells under blue light. As the positive control, WT CAR T-cells showed light-independent killing of tumor cells. By contrast, defective LiCAR T-cells did not trigger tumor killing even under photo-illumination.

FIG. 12. The growth rates of B16-OVA and B16-OVA-hCD19 cells in vitro and in vivo. (related to FIG. 3) a, In vitro cell proliferation of B16-OVA-hCD19 and B16-OVA cells quantified using the WST-1 colorimetric assay. Absorbance at 450 nm was used as readout. n=6 (mean±s.e.m.) b, The growth curves of 5×10⁵ B16-OVA-hCD19 and B16-OVA melanoma cells after intradermal injection into the left and right flanks, respectively, of C57BL/6J mice. Tumor sizes at the indicated time points were measured by a digital caliper with the tumor areas calculated in mm² (length×width). No significant difference in tumor sizes were noted. P=0.686 when compared to the hCD19-negative B16-OVA group at day 19 (paired two-tailed Student's t-test; n=9; mean±s.e.m.)

FIG. 13. Optimization and characterization of synthesized UCNPs. (related to FIG. 3c-f) a, Comparison of the the core/shell structures of synthesized UCNPs (top, β-NaYF₄:30% Yb, 0.5% Tm@NaYF₄; bottom, β-NaYbF₄:0.5% Tm@NaYF4) and representative TEM images (right). Scale bar, 100 nm. b, Representative TEM images and the size distribution (height denoted as “H” and diameter denoted as “D”) of the NaYbF₄:0.5% Tm core nanoparticles (left), NaYbF₄:0.5% Tm@NaYF₄ core-shell nanoplates (middle), and silica-coated NaYbF₄:0.5% Tm@NaYF4 core-shell nanoplates (right). c, Comparison of the upconversion luminescence spectra of synthesized UCNPs upon MR light illumination at 980 nm (black, β-NaYF4:30% Yb,0.5% Tm@NaYF4; red, β-NaYbF₄:0.5% Tm@NaYF4 nanoplates). Their luminescence intensities were compared at the same condition with the same amounts of total lanthanide ions. d, Blue light emitting from the leftmost cuvette containing UCNPs (β-NaYbF₄:0.5% Tm@NaYF4) upon MR illumination. The UCNP-containing cuvette (leftmost) was placed next to the indicated numbers of H₂O-containing plastic cuvettes (labeled as 1, 2 and 3; top) or a cuvette containing dark ink (bottom). The MR light source (980 nm) was placed on the right. Pictures were taken in a dark room except for the leftmost images. The light intensity was strong enough to illuminate the background after penetrating through cuvettes. e, Injectable UCNPs emitted bright blue light locally at the injection site in vivo upon MR light stimulation (980 nm; 250 mW/cm²). Pictures were taken for the same mouse in the bright field without (left) or with MR light (middle), or in the dark field with MR light (right).

FIG. 14. In vivo biosafety and biocompatibility evaluation of silica-coated UCNPs. Scale bar, 100 μm. (related to FIGS. 3-5) a, B16-OVA-mCD19 cell viability assessed by an MTT assay upon incubation with different concentrations of UCNPs. b-c, Typical H&E staining images of major organs (heart, liver, spleen, lung, kidney and tumor) isolated from UCNPs (1 mg/ml, 150 μL) or PBS-injected (150 μL at tumor sites) tumor-bearing mice (b) and healthy mice (c). d-e, The percentage of total, M1 and M2 macrophages in the tumor (d) and spleen (e) isolated from UCNP- or PBS-injected mice.

FIG. 15. Effects of WT CAR T-cells and UCNPs on tumor growth. (Related to FIG. 3f-g) a, WT CAR-expressing CD8⁺ T-cells selectively destroy CD19-positive melanoma tumors without light stimulation. Left, C57BL/6J mice were intradermally inoculated with 2.5×10⁵ B16-OVA-hCD19 cells in the left flank (red circle) and 2.5×10⁵ B16-OVA cells (CD19-negative tumor as control; blue circle) in the right flank. Two representative mice are shown after treatment with WT CAR T-cells+UCNPs for 8 days. Middle, Tumor sizes at the indicated time points were measured by a digital caliper with the tumor areas calculated in mm² (length x width). *P<0.05 when compared to the CD19-negative B16-OVA group at day 18 (paired two tailed Student's t-test; n=4; mean±s.e.m.). Right, isolated B16-OVA and B16-OVA-hCD19 tumors at day 18. b, The growth curves of B16-OVA-CD19/UCNPs and B16-OVA/UCNPs upon MR light irradiation. Left, C57BL/6J mice were intradermally inoculated with 2.5×10⁵ B16-OVA-hCD19 cells in the left flank (red circle) and 2.5×10⁵ B16-OVA cells (CD19-negative tumor as control; blue circle) in the right flank. Two representative mice are shown after injection of UCNPs for 8 days without CAR T-cells under MR treatment. Middle, Tumor sizes at the indicated time points were measured by a digital caliper with the tumor areas calculated in mm² (length x width). No significant difference in tumor size were observed. P=0.93 when compared to the B16-OVA group at day 16 (paired two-tailed Student's t-test; n=3; mean±s.e.m.). Right, isolated B16-OVA and B16-OVA-hCD19 tumors at day 16. c, UCNPs did not affect tumor growth. C57BL/6J mice were intradermally inoculated with 2.5×10⁵ B16-OVA cells to each flank. Four representative mice were shown after injection with UCNPs to the right flank tumor. Top right, isolated B16-OVA and B16-OVA-UCNPs tumors before and after UCNP removals at day 18. Bottom left and middle, tumor sizes before tumor surgery (measured from outside the skin) and after UCNP removal, respectively, at day 18 were measured by a digital caliper with the tumor areas calculated in mm² (length×width). Bottom right, tumor weight after UCNPs removal at day 18. No significant difference in tumor sizes or weights were observed.

FIG. 16. Light-inducible selective killing of CD19⁺ solid tumors in vivo using LiCAR-expressing T cells. (related to FIG. 3f-h) a, CD8³⁰ LiCAR T-cells selectively destroy CD19-expressing melanoma in response to MR light illumination. Left, C57BL/6J mice were intradermally inoculated with 2.5×10⁵ B16-OVA-hCD19 cells in the left flank and 2.5×10⁵ B16-OVA cells (CD19-negative tumor as control) in the right flank. Two representative mice with opened tumor areas are shown after treatment with LiCAR T-cells+UCNPs and exposure to MR pulses for 9 days. Right, isolated B16-OVA/UCNPs and B16-OVA-hCD19/UCNPs tumors at day 19. Green arrow, tumor cells. Blue arrow, UCNPs injected to tumor sites. The tumor masses after UCNP removal were shown in FIG. 3g. b, LiCAR T-cells permit MR light-inducible killing of B16-OVA-hCD19 melanoma in selected regions. Left, C57BL/6J mice were intradermally inoculated at both flanks with 3×10⁵ B16-OVA-hCD19 cells. After injection with the LiCAR T-cells+UCNP mixture, the left flank was exposed to MR pulses for 8 days, while the right side was protected from MR light using aluminum foil. Two representative mice with opened tumors are shown at day 18. Right, isolated B16-OVA-hCD19/UCNPs tumors with and without MR at day 18. Green arrow, tumor cells. Blue arrow, UCNPs injected to tumor sites. The tumor masses after UCNP removal were shown in FIG. 3h.

FIG. 17. UCNPs are well confined within the injection site. (related to FIGS. 3-5) a, Visualizing UCNPs after s.c. injection into the tumor sites under the indicated conditions. Images were acquired in the same mouse under three conditions: bright field without MR light (left), bright field with MR light illumination (middle) at the injection site (red arrow), or in the dark room with MR light (right). Top, in situ images; middle, the UCNP-containing skin tissues were surgically exposed; bottom, after surgical removal. The MR excitation showed a pink color in the camera if blue emission was not detected. Zoomed-in view on the right (orange box): The UCNP-containing skin/tumor tissues (top) and well-confined UCNPs isolated from the tissue (bottom). b, UCNPs did not spread to other major organs within the experimental window. Major organs were isolated from the mouse shown in panel a and then subjected to MR light illumination. Only pink color was noted without blue emission, suggesting the absence of UCNPs in these tissues.

FIG. 18. Evolution of the stability of UCNPs in vivo up to 28 days. a, TEM images showing the morphology of UCNPs at different time points after injection in tumor-bearing mice (day 1, day 7 and day 14 [end point when tumor reaching the maximally allowed size by IACUC]) or the leg muscle of healthy mice (day 28). b, UCNPs showing bright emission under 980 nm laser excitation at 28 days after being injected into the leg muscle of mice. Other organs without UCNPs were used as the controls.

FIG. 19. Element analysis of the nanoparticle distribution in living tissues using an energy-dispersive X-ray spectroscopy (EDS) coupled SEM system. (Related to FIG. 3) a-g, The element analysis of the UCNP injected tumor, tumor-surrounding tissues, as well as other major organs (heart, liver, spleen, lung and kidney; as shown in FIG. 17) from the UCNP-injected mouse. Top, SEM images of the indicated tissues (left); the element mapping showing the major three elements of the sample (middle); and the corresponding backscattered electron images (right; Note: heavy atoms such as lanthanides gave strong signals, while light atoms remained dim in the image); Bottom, the energy-dispersive X-ray spectrum of the indicated tissue samples.

FIG. 20. LiCAR T-cells retention and expansion in vivo. (related to FIG. 3g-h) a, Schematic of the in vivo experimental setup for LiCAR T-cell isolation from mice bearing melanoma tumors. Left, B16-OVA-hCD19 inoculation, without LiCAR T-cells+UCNPs and MR treatment (as mock control); Right, B16-OVA hCD19 (left flank) and B16-OVA inoculation (right flank), with LiCAR T-cells+UCNPs and MR treatment. b, FACS analysis of LiCAR T-cells (GFP+/mCherry+) within the isolated tumor masses at day 19 (shown in FIG. 16a). LiCAR T-cells were mostly detected in the B16-OVA-hCD19 tumor, but were not readily detectable in the mock and B16-OVA tumors (n=five mice). c, Monitoring the off-tumor distribution of LiCAR T-cells by flow cytometry. Nominal GFP+/mCherry+signals were detected in the spleen and peripheral blood isolated from the B16-OVA-hCD19 group. d, FACS analysis of LiCAR T-cells within the isolated tumor masses at day 18 (shown in FIG. 16b). LiCAR T-cells were more abundantly detected in the tumors exposed to MR light (from four mice) when compared to the mock and the non-MR treated tumors (n=five mice), indicating more robust activation/expansion of LiCAR T-cells after MR-inducible reconstitution of functional CARs.

FIG. 21. Blue light did not induce statistically significant changes in tumor killing. (related to FIG. 3g-i) a, Cartoon illustrating the experimental setup. C57BL/6J mice were intradermally inoculated in the left flank with 3×10⁵ B16-OVA-hCD19 cells. After injection with the LiCAR T-cells+UCNP mixture, mice were either exposed to blue light (470 nm; 40 mW/cm², 2 hours per day) for 8 days, or kept in the dark. b, Three representative mice from each group. The lower panel showed mice with opened tumors at day 18. Green arrow, tumor injection sites. Blue arrow, UCNPs in the tumor sites. c, Measurements of tumor sizes at the indicated time points by a digital caliper. Tumor areas were calculated in mm² (length×width). P=0.25 when compared to the dark group at day 18 (paired two-tailed Student's t-test; mean±s.e.m.). d, Representative images of isolated B16-OVA-hCD19 tumors (shown in panel b) with and without blue light at day 18.

FIG. 22. Validation of melanoma cells expressing mouse CD19 (mCD19) antigen and quantification of B cell population in mCAR or mLiCAR-treated mice by flow cytometry. (related to FIG. 4) a, Quantification of mCD19 expression in melanoma cells (B16-OVA) and melanoma cells exogenously expressing mouse CD19 (B16-OVA-mCD19). b, Representative peripheral blood B cell populations from WT mCAR T-cells/UCNPs (FIG. 4c) or mLiCAR T-cells/UCNPs/NIR pulses (FIG. 4d)-treated mice bearing B16-OVA/B16-OVA-mCD19 tumors on day 0 and day 3. B cells from peripheral blood of healthy mice were used as a control for normal B cell counts. B cells isolated from spleens were stained with anti-CD19-APC (positive control) and non-stained B cells (negative control) were used to aid in the gating of the cell populations (top panels).

### Definitions

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “Chimeric Antigen Receptor” or alternatively a “CAR” refers to a recombinant polypeptide construct comprising at least an extracellular antigen binding domain, a transmembrane domain and a cytoplasmic signaling domain comprising a functional signaling domain derived from a stimulatory molecule as defined below. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from 4 1BB (i.e., CD137) and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

As used herein, a “signaling domain” is the functional portion of a protein which acts by transmitting information within the cell to regulate cellular activity via defined signaling pathways by generating second messengers or functioning as effectors by responding to such messengers.

The term “antibody,” as used herein, refers to an immunoglobulin molecule which specifically binds with an antigen. Antibodies can be polyclonal or monoclonal, multiple or single chain, or intact immunoglobulins, and may be derived from natural sources or from recombinant sources. Antibodies such as IgG are typically tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multispecific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of the CAR composition of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

An “antibody heavy chain,” as used herein, refers to the larger of the two types of polypeptide chains present in antibody molecules in their naturally occurring conformations, and which normally determines the class to which the antibody belongs

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

The term “antigen” or “Ag” as used herein is defined as a molecule that can provoke an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or can be macromolecules besides a polypeptide. Such a biological sample can include, but is not limited to a tissue sample, a tumor sample, a cell or fluid with other biological components.

The term “anti-tumor effect” as used herein, refers to a biological effect which can be manifested by various means, including but not limited to, a decrease in tumor volume, a decrease in the number of tumor cells, a decrease in the number of metastases, an increase in life expectancy, a decrease in tumor cell proliferation, a decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to whom it is later to be re-introduced into the individual.

As used herein, the term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

By the term “stimulation,” used in the context of CART, is meant a primary response induced by binding of a receptor complex (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” used in the context of CART, means a molecule expressed by a T cell that provide the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MEC molecule loaded with peptide (such as tumor antigen), and which leads to mediation of a T cell response, including, but not limited to, proliferation, activation, differentiation, and the like. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, CD278 (also known as “ICOS”) and CD66d.

An “antigen presenting cell,” as used herein, means an immune system cell such as an accessory cell (e.g., a B-cell, a dendritic cell, and the like) that displays foreign antigens complexed with major histocompatibility complexes (WIC's) on their surfaces. T-cells may recognize these complexes using their T-cell receptors (TCRs). APCs process antigens and present them to T-cells.

A “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18) and 4-1BB (CD137).

As used herein “4-1BB” is defined as member of the TNFR superfamily with an amino acid sequence provided as GenBank accno. AAA62478.2, or the equivalent residues from a non-human species, e.g., mouse, rodent, monkey, ape and the like; and a “4-1BB costimulatory domain” are defined amino acid residues 214-255 of GenBank accno. AAA62478.2, or the equivalent residues from a non-human species, e.g., mouse, rodent, monkey, ape and the like.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

A “transfer vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, polylysine compounds, liposomes, and the like. Examples of viral transfer vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared x100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994))

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “specifically binds,” as used herein with respect to an antibody, is meant an antibody or antibody fragment which recognizes and binds with a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DETAILED DESCRIPTION OF THE INVENTION

The invention is based in part on the discovery of a novel, highly efficient and robust photolysis platform methodology where

Disclosed herein is the engineering of light sensitivity into the chimeric antigen receptors to create light-switchable CAR (LiCAR) T-cells. LiCAR-expressing T-cells can selectively produce anti-tumor immune responses in the dual presence of tumor antigen and light. To demonstrate the feasibility of such wireless optogenetic intervention in vivo, we further combined LiCAR with surgically removable upconversion nanoplates (UCNPs) that have enhanced near infrared (NIR)-to-blue upconversion luminescence. The UCNPs act as miniature light transducers that allow inducible activation of CAR T-cells in living animals to occur upon stimulation with deep tissue-penetrable MR light. This MR light-tunable nano-optogenetic immunomodulation platform enables spatiotemporal control of CAR T-cell mediated cytotoxicity against both hematological malignancies and solid tumors with tailored doses and duration, thereby greatly mitigating side effects associated with the current immunotherapy.

For example, a proof-of-concept study is disclosed herein that focuses on targeting the CD19 antigen overexpressed in certain types of tumors. We have demonstrated the successful design of photoswitchable CARs to deliver dual input (antigen+photon)-gated immune response using engineered therapeutic T cells. LiCAR enables light control over the therapeutic activity of CAR T-cells by simply varying the duration of light illumination in vitro. Depending on the readouts and various optical dimerizers used in our studies, we observed some minor leakage in the dark with some LiCAR constructs (e.g., C+D4). This problem has been partially solved by using improved optical dimerizers with weaker affinities, and hence reduced interactions in the dark. Admittedly, the weaker versions (C+D4.1, or C+D4.3) cause less potent activation of re-assembled CAR T-cells, but have the advantage of minimized pre-activation to confer tight control over engineered CAR T-cells. Collectively, we provide herein a set of LiCAR tools tailored for different purposes. For potent activation, the combination of C+D4 is the best choice; whereas for more strict control, the C+D4.1 or C+D4.3 combinations will likely serve as better options. Worthy to note, the background activity of all three combinations was deemed as minor or negligible since they are unable to trigger cytokine secretion of T cells or its cytotoxic functions. Congruently, with these three options, we believe that stronger and weaker versions of LiCAR variants provide a more dynamic system for tunable control of engineered immune cells.

In conjunction with injectable and imaging guided post-treatment removable upconversion nanoplates, LiCAR permits time- and location-specific CAR T cell-mediated antitumor activity via deep tissue penetrable MR light in vivo. This hybrid strategy allows for the precise spatiotemporal regulation of the T cell-mediated immune response, as well as mitigating the side effects associated with immunotherapy, such as “on-target, off-tumor” cytotoxicity and CRS toxicity. Therefore, we anticipate that it can ultimately lead to the development of new generations of personalized optogenetic immunotherapy, where the timing, location, and dosage of T cell-mediated therapeutic activity can be tailored to the patients' needs.

In one aspect, the invention generally relates to an isolated nucleic acid sequence or isolated nucleic acid sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a CAR.

In certain embodiments of the isolated nucleic acid sequence or sequences, the first part of the optogenetic dimerizer pair is fused with one of the at least one first costimulatory domain.

In certain embodiments, the first part of the optogenetic dimerizer pair is fused with one of the at least one extracellular antigen binding domain.

In certain embodiments of the isolated nucleic acid sequence or sequences, the second part of the optogenetic dimerizer pair is fused with one of the at least one second costimulatory domain.

In certain embodiments, the second part of the optogenetic dimerizer pair is fused with the intracellular signaling domain.

Any suitable optogenetic dimerizer pairs may be employed. In certain embodiments, the optogenetic dimerizer pair may be selected from the group consisting of: CRY2/CIBN pair, LOV2-ssrA/sspB pair or its modified version using circularly permuted LOV2 (cpLOV2), pMag, CRY2/SPA1 pair, and CRY2/BIC1 pair. (Kawano, et al. 2015 Nature Comm., 6:6256; DOI: 10.1038/ncomms7256; SPA1 (suppressor of phyA-105) protein family, Gene ID: 819242; Protein BIC1, https://www.uniprot.org/uniprot/Q9LXJ1.)

In certain embodiments, the optogenetic dimerizer pair is the CRY2/CIBN pair.

In certain embodiments, the optogenetic dimerizer pair is the LOV2-ssrA/sspB pair.

The selection of the optogenetic dimerizer pair may be considered in view of the application at hand. In certain embodiments, the optogenetic dimerizer pair fuses upon excitation of a light having a wavelength in the range of about 400 nm to about 1,000 nm (e.g., about 500 nm to about 1,000 nm, about 600 nm to about 1,000 nm, about 700 nm to about 1,000 nm, about 800 nm to about 1,000 nm, about 400 nm to about 900 nm, about 400 nm to about 800 nm, about 400 nm to about 700 nm, about 400 nm to about 600 nm). In certain embodiments, the optogenetic dimerizer pair fuses upon excitation of a light having a wavelength in the range of about 450 nm to about 700 nm (e.g., about 500 nm to about 700 nm, about 550 nm to about 700 nm, about 600 nm to about 700 nm, about 450 nm to about 650 nm, about 450 nm to about 600 nm, about 450 nm to about 550 nm).

In the isolated nucleic acid sequence or sequences disclosed herein, the at least one extracellular antigen binding domain binds to an antigen expressed on a target cell (e.g., a tumor or cancer cell).

In certain embodiments, the at least one extracellular antigen binding domain binds to an antigen expressed on a tumor cell.

In certain embodiments, the at least one extracellular antigen binding domain binds to an antigen expressed on a cancer cell.

In certain embodiments, the tumor or cancer cell is that of a liquid tumor or cancer, e.g., selected from the group consisting of: leukemia and lymphoma. In certain embodiments, the liquid tumor or cancer is selected from the group consisting of: K562, Raji and Daudi cancer cells.

In certain embodiments, the tumor or cancer cell is that of a solid tumor or cancer, e.g., selected from the group consisting of: glioma, astrocytoma, skin cancer, breast cancer, prostate cancer, colorectal cancer, renal cancer, stomach cancer, bladder cancer, pancreatic cancer, hepatocarcinoma, lung cancer, endometrial cancer and thyroid cancer. In certain embodiments, the solid tumor or cancer is selected from the group consisting of: melanoma and glioblastoma.

In certain embodiments of the isolated nucleic acid sequence or sequences disclosed herein, the transmembrane domain comprising a transmembrane domain of a protein selected from the group consisting of the T-cell receptor (TCR) alpha chain, the TCR betachain, the TCR zeta chain, CD28, CD3 epsilon, CD45, CD4, CD5, CD8, CD9, CD16, CD22, CD33, CD37, CD64, CD80, CD86, CD134, CD137, CD154, or any combination thereof.

In certain embodiments of the isolated nucleic acid sequence or sequences disclosed herein, the at least one costimulatory domain comprising a functional signaling domain selected from the group consisting of OX40, CD70, CD27, CD28, CD5, ICAM-1, LFA-1 (CD11a/CD18), ICOS (CD278), DAP10, DAP12, 4-1BB (CD137), or any combination thereof.

In certain embodiments of the isolated nucleic acid sequence or sequences disclosed herein, the intracellular signaling domain comprising a functional domain selected from the group consisting of a 4-1BB (CD137); CD28, and CD3 zeta signaling domain, or a combination thereof.

In certain embodiments, the at least one extracellular antigen binding domain is connected to the transmembrane domain by a linker or spacer domain.

In certain embodiments of the isolated nucleic acid sequence or sequences disclosed herein, the at least one extracellular antigen binding domain consists of a single extracellular antigen binding domain. In certain embodiments of the isolated nucleic acid sequence or sequences disclosed herein, the at least one extracellular antigen binding domain comprise two or more (e.g., 2, 3, 4) extracellular antigen binding domains.

In certain embodiments, the isolated nucleic acid sequence or sequences consist of one isolated nucleic acid sequence comprising the first nucleic acid sequence encoding Component I and the second nucleic acid sequence encoding Component II.

In certain embodiments, the isolated nucleic acid sequence or sequences comprise a first isolated nucleic acid sequence comprising the first nucleic acid sequence encoding Component I and a second isolated nucleic acid sequence comprising the second nucleic acid sequence encoding Component II.

In another aspect, the invention generally relates to a vector that comprises a nucleic acid sequence or nucleic acid sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a CAR.

In certain embodiments of the vector disclosed herein, the first part of an optogenetic dimerizer pair is fused with one of the at least one first costimulatory domain.

In certain embodiments, the first part of an optogenetic dimerizer pair is fused with one of the at least one extracellular antigen binding domain.

In certain embodiments of the vector disclosed herein, the second part of the optogenetic dimerizer pair is fused with one of the at least one second costimulatory domain.

In certain embodiments, the second part of the optogenetic dimerizer pair is fused with the intracellular signaling domain.

As discussed herein, any suitable optogenetic dimerizer pairs may be employed. In certain embodiments, the optogenetic dimerizer pair is selected from the group consisting of: CRY2/CIBN pair, LOV2-ssrA/sspB pair or its modified version using circularly permuted LOV2 (cpLOV2), pMag, CRY2/SPA1 pair, and CRY2/BIC1 pair. In certain embodiments, the optogenetic dimerizer pair is the CRY2/CIBN pair. In certain embodiments, the optogenetic dimerizer pair is the LOV2-ssrA/sspB pair.

The selection of the optogenetic dimerizer pair may be considered in view of the application at hand. In certain embodiments, the optogenetic dimerizer pair fuses upon excitation of a light having a wavelength in the range of about 400 nm to about 1,000 nm (e.g., about 500 nm to about 1,000 nm, about 600 nm to about 1,000 nm, about 700 nm to about 1,000 nm, about 800 nm to about 1,000 nm, about 400 nm to about 900 nm, about 400 nm to about 800 nm, about 400 nm to about 700 nm, about 400 nm to about 600 nm). In certain embodiments, the optogenetic dimerizer pair fuses upon excitation of a light having a wavelength in the range of about 450 nm to about 700 nm (e.g., about 500 nm to about 700 nm, about 550 nm to about 700 nm, about 600 nm to about 700 nm, about 450 nm to about 650 nm, about 450 nm to about 600 nm, about 450 nm to about 550 nm).

In certain embodiments of the vector disclosed herein, the at least one extracellular antigen binding domain binds to an antigen expressed on a target cell. The target cell may be any suitable cell, e.g., is a tumor cell. In certain embodiments, the tumor cell is a cancer cell (carcinoma). The tumor or cancer cell can be a liquid tumor or cancer (e.g., selected from the group consisting of: leukemia and lymphoma) or a solid tumor or cancer (e.g., selected from the group consisting of: glioma, astrocytoma, skin cancer, breast cancer, prostate cancer, colorectal cancer, renal cancer, stomach cancer, bladder cancer, pancreatic cancer, hepatocarcinoma, lung cancer, endometrial cancer and thyroid cancer).

In certain embodiments of the vector disclosed herein, the transmembrane domain comprising a transmembrane domain of a protein selected from the group consisting of the T-cell receptor (TCR) alpha chain, the TCR betachain, the TCR zeta chain, CD28, CD3 epsilon, CD45, CD4, CD5, CD8, CD9, CD16, CD22, CD33, CD37, CD64, CD80, CD86, CD134, CD137, CD154, or any combination thereof.

In certain embodiments of the vector disclosed herein, the at least one costimulatory domain comprising a functional signaling domain selected from the group consisting of OX40, CD70, CD27, CD28, CD5, ICAM-1, LFA-1 (CD11a/CD18), ICOS (CD278), DAP10, DAP12, 4-1BB (CD137), or any combination thereof.

In certain embodiments, the intracellular signaling domain comprising a functional domain selected from the group consisting of a 4-1BB (CD137); CD28, and CD3 zeta signaling domain, or a combination thereof.

In certain embodiments, the at least one extracellular antigen binding domain is connected to the transmembrane domain by a linker or spacer domain.

In certain embodiments of the vector disclosed herein, the nucleic acid sequence or nucleic acid sequences consist of one isolated nucleic acid sequence comprising the first nucleic acid sequence encoding Component I and the second nucleic acid sequence encoding Component II. In certain embodiments, the nucleic acid sequence or nucleic acid sequences comprise a first isolated nucleic acid sequence comprising the first nucleic acid sequence encoding Component I and a second isolated nucleic acid sequence comprising the second nucleic acid sequence encoding Component II.

In yet another aspect, the invention generally relates to a cell comprising a nucleic acid sequence or sequences, which comprise: a first nucleic acid sequence encoding at least one extracellular antigen binding domain, a transmembrane domain, at least one first costimulatory domain, and a first part of an optogenetic dimerizer pair (collectively “Component I”); and a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a CAR.

In certain embodiments of the cell disclosed herein, the cell is selected from the group consisting of a T cell, a natural killer (NK) cell, a cytotoxic T lymphocyte (CTL), a regulatory T cell, and macrophage. In certain embodiments, the cell is a T cell. In certain embodiments, a natural killer (NK) cell. In certain embodiments, a cytotoxic T lymphocyte (CTL). In certain embodiments, a regulatory T cell.

In certain embodiments of the cell disclosed herein, the first part of an optogenetic dimerizer pair is fused with one of the at least one first costimulatory domain.

In certain embodiments, the first part of an optogenetic dimerizer pair is fused with one of the at least one extracellular antigen binding domain.

In certain embodiments of the cell disclosed herein, the second part of the optogenetic dimerizer pair is fused with one of the at least one second costimulatory domain.

In certain embodiments, the second part of the optogenetic dimerizer pair is fused with the intracellular signaling domain.

As discussed herein, any suitable optogenetic dimerizer pairs may be employed. In certain embodiments, the optogenetic dimerizer pair is selected from the group consisting of: CRY2/CIBN pair, LOV2-ssrA/sspB pair or its modified version using circularly permuted LOV2 (cpLOV2), pMag, CRY2/SPA1 pair, and CRY2/BIC1 pair. In certain embodiments, the optogenetic dimerizer pair is the CRY2/CIBN pair. In certain embodiments, the optogenetic dimerizer pair is the LOV2-ssrA/sspB pair.

The selection of the optogenetic dimerizer pair may be considered in view of the application at hand. In certain embodiments, the optogenetic dimerizer pair fuses upon excitation of a light having a wavelength in the range of about 400 nm to about 1,000 nm (e.g., about 500 nm to about 1,000 nm, about 600 nm to about 1,000 nm, about 700 nm to about 1,000 nm, about 800 nm to about 1,000 nm, about 400 nm to about 900 nm, about 400 nm to about 800 nm, about 400 nm to about 700 nm, about 400 nm to about 600 nm). In certain embodiments, the optogenetic dimerizer pair fuses upon excitation of a light having a wavelength in the range of about 450 nm to about 700 nm (e.g., about 500 nm to about 700 nm, about 550 nm to about 700 nm, about 600 nm to about 700 nm, about 450 nm to about 650 nm, about 450 nm to about 600 nm, about 450 nm to about 550 nm).

In certain embodiments of the vector disclosed herein, the at least one extracellular antigen binding domain binds to an antigen expressed on a target cell. The target cell may be any suitable cell, e.g., is a tumor cell. In certain embodiments, the tumor cell is a cancer cell (carcinoma). The tumor or cancer cell can be a liquid tumor or cancer (e.g., selected from the group consisting of: leukemia and lymphoma or a solid tumor or cancer (e.g., selected from the group consisting of: glioma, astrocytoma, skin cancer, breast cancer, prostate cancer, colorectal cancer, renal cancer, stomach cancer, bladder cancer, pancreatic cancer, hepatocarcinoma, lung cancer, endometrial cancer and thyroid cancer.

In certain embodiments of the vector disclosed herein, the transmembrane domain comprising a transmembrane domain of a protein selected from the group consisting of the T-cell receptor (TCR) alpha chain, the TCR betachain, the TCR zeta chain, CD28, CD3 epsilon, CD45, CD4, CD5, CD8, CD9, CD16, CD22, CD33, CD37, CD64, CD80, CD86, CD134, CD137, CD154, or any combination thereof.

In certain embodiments of the vector disclosed herein, the at least one costimulatory domain comprising a functional signaling domain selected from the group consisting of OX40, CD70, CD27, CD28, CD5, ICAM-1, LFA-1 (CD11a/CD18), ICOS (CD278), DAP10, DAP12, 4-1BB (CD137), or any combination thereof.

In certain embodiments, the intracellular signaling domain comprising a functional domain selected from the group consisting of a 4-1BB (CD137); CD28, and CD3 zeta signaling domain, or a combination thereof.

In certain embodiments, the at least one extracellular antigen binding domain is connected to the transmembrane domain by a linker or spacer domain.

In certain embodiments of the vector disclosed herein, the nucleic acid sequence or nucleic acid sequences consist of one isolated nucleic acid sequence comprising the first nucleic acid sequence encoding Component I and the second nucleic acid sequence encoding Component II. In certain embodiments, the nucleic acid sequence or nucleic acid sequences comprise a first isolated nucleic acid sequence comprising the first nucleic acid sequence encoding Component I and a second isolated nucleic acid sequence comprising the second nucleic acid sequence encoding Component II.

In yet another aspect, the invention generally relates to a pharmaceutical composition comprising a cell population comprising an anti-tumor effective amount of a cell disclosed herein.

In certain embodiments of the pharmaceutical composition disclosed herein, the cell population is that of a T cell.

In certain embodiments, the pharmaceutical composition is suitable for one or more of intramuscular, subcutaneous, intravenous, and intradermal injection.

In yet another aspect, the invention generally relates to a UCNP having a core-shell structure, which comprises: a core comprising β-NaYbF₄:Tm; and a shell comprising β-NaYF₄.

In certain embodiments of the UCNP disclosed herein, wherein the UCNP is hexagonal shaped. In certain embodiments, the UCNP is plate shaped.

In certain embodiments, the UCNP core is hexagonal-plate shaped. In certain embodiments, the hexagonal-plate shaped core has a dimension in the range of about 1 nm to about 5,000 nm (e.g., about 10 nm to about 5,000 nm, about 100 nm to about 5,000 nm, about 200 nm to about 5,000 nm, about 500 nm to about 5,000 nm, about 1,000 nm to about 5,000 nm, about 1 nm to about 1,000 nm, about 1 nm to about 500 nm, about 1 nm to about 300 nm, about 1 nm to about 100 nm, about 1 nm to about 50 nm, about 10 nm to about 1,000 nm, about 50 nm to about 500 nm, about 100 nm to about 500 nm) in diameter and about 1 nm to about 5,000 nm (e.g., about 10 nm to about 5,000 nm, about 100 nm to about 5,000 nm, about 200 nm to about 5,000 nm, about 500 nm to about 5,000 nm, about 1,000 nm to about 5,000 nm, about 1 nm to about 1,000 nm, about 1 nm to about 500 nm, about 1 nm to about 300 nm, about 1 nm to about 100 nm, about 1 nm to about 50 nm, about 10 nm to about 1,000 nm, about 50 nm to about 500 nm, about 100 nm to about 500 nm) in height.

In certain embodiments, the hexagonal-plate shaped core has a dimension in the range of about 1 nm to about 200 nm (e.g., about 5 nm to about 200 nm, about 10 nm to about 200 nm, about 20 nm to about 200 nm, about 50 nm to about 200 nm, about 100 nm to about 200 nm, about 1 nm to about 100 nm, about 1 nm to about 50 nm, about 1 nm to about 20 nm, about 1 nm to about 10 nm) in diameter and about 1 nm to about 200 nm (e.g., about 5 nm to about 200 nm, about 10 nm to about 200 nm, about 20 nm to about 200 nm, about 50 nm to about 200 nm, about 100 nm to about 200 nm, about 1 nm to about 100 nm, about 1 nm to about 50 nm, about 1 nm to about 20 nm, about 1 nm to about 10 nm) in height.

In certain embodiments, the shell comprising β-NaYF4is characterized by a thickness in the range of about 1 nm to about 1,000 nm (e.g., about 5 nm to about 1,000 nm, about 10 nm to about 1,000 nm, about 50 nm to about 1,000 nm, about 100 nm to about 1,000 nm, about 200 nm to about 1,000 nm, about 500 nm to about 1,000 nm, about 1 nm to about 500 nm , about 1 nm to about 200 nm, about 1 nm to about 100 nm, about 1 nm to about 50 nm, about 1 nm to about 20 nm). In certain embodiments, the shell comprising β-NaYF4is characterized by a thickness in the range of about 1 nm to about 50 nm (e.g., about 2 nm to about 50 nm, about 5 nm to about 50 nm, about 10 nm to about 50 nm, about 20 nm to about 50 nm, about 1 nm to about 20 nm, about 1 nm to about 10 nm, about 1 nm to about 5 nm, about 5 nm to about 20 nm).

In certain embodiments, the UCNP further comprises an outer shell of silica. In certain embodiments, the silica outer shell is characterized by a thickness in the range of about 1 nm to about 1,000 nm (e.g., 1 nm to about 1,000 nm, 5 nm to about 1,000 nm, 10 nm to about 1,000 nm, 50 nm to about 1,000 nm, 100 nm to about 1,000 nm, 500 nm to about 1,000 nm, 1 nm to about 500 nm, 1 nm to about 200 nm, 1 nm to about 100 nm, 1 nm to about 50 nm, 1 nm to about 20 nm, 1 nm to about 10 nm). In certain embodiments, the silica outer shell is characterized by a thickness in the range of about 1 nm to about 50 nm (e.g., about 2 nm to about 50 nm, about 5 nm to about 50 nm, about 10 nm to about 50 nm, about 20 nm to about 50 nm, about 1 nm to about 20 nm, about 1 nm to about 10 nm, about 1 nm to about 5 nm, about 5 nm to about 20 nm).

In certain embodiments of the UCNP disclosed herein, Yb accounts for a mol% from about 1% to about 99% (e.g., from about 10% to about 99%, from about 30% to about 99%, from about 50% to about 99%, from about 70% to about 99%, from about 80% to about 99%, from about 90% to about 99%, from about 95% to about 99%, from about 1% to about 90%, from about 1% to about 50%, from about 1% to about 30%, from about 1% to about 20%, from about 1% to about 10%, from about 1% to about 5%) of the core; and Tm accounts for a mol% from about 0.1% to about 99.9% (e.g., from about 10% to about 99.9%, from about 30% to about 99.9%, from about 50% to about 99.9%, from about 70% to about 99.9%, from about 80% to about 99.9%, from about 90% to about 99.9%, from about 95% to about 99.9%, from about 1% to about 95%, from about 1% to about 90%, from about 1% to about 80%, from about 1% to about 50%, from about 1% to about 30%, from about 1% to about 10%, from about 1% to about 5%) of the core. In certain embodiments, Yb accounts for a mol% from about 1% to about 90% of the core; and Tm accounts for a mol% from about 0.1% to about 90% of the core.

In certain embodiments, the UCNP is characterized by a constitutional excitation spectrum comprising a peak in the range of about 700 nm to about 1,100 nm (e.g., about 800 nm to about 1,100 nm, about 900 nm to about 1,100 nm, about 1,000 nm to about 1,100 nm, about 700 nm to about 1,000 nm, about 700 nm to about 900 nm, about 700 nm to about 800 nm, about 800 nm to about 1,000 nm). In certain embodiments, the UCNP is characterized by a constitutional excitation spectrum comprising a peak in the range of about 800 nm to about 1,000 nm.

In certain embodiments, the UCNP is characterized by an emission spectrum comprising a peak in the range of about 380 nm to about 700 nm (e.g., about 450 nm to about 700 nm, about 500 nm to about 700 nm, about 550 nm to about 700 nm, about 600 nm to about 700 nm, about 380 nm to about 650 nm, about 380 nm to about 600 nm, about 380 nm to about 550 nm, about 380 nm to about 500 nm). In certain embodiments, the UCNP is characterized by an emission spectrum comprising a peak in the range of about 380 nm to about 500 nm.

In certain embodiments, the UNCP is biocompatible.

In yet another aspect, the invention generally relates to a pharmaceutical composition comprising a UCNP disclosed herein.

In yet another aspect, the invention generally relates to a pharmaceutical composition comprising a herein disclosed UCNP and a disclosed herein cell.

In certain embodiments, the herein disclosed pharmaceutical composition is suitable for one or more of intramuscular, subcutaneous, intravenous, and intradermal injection.

In yet another aspect, the invention generally relates to a method for activating a light sensitive biological agent or cell. The method comprises: administering to a subject in need thereof a UCNP and a light sensitive biological agent or cell; exciting the UCNP with an excitation light to cause the UCNP to emit a luminescence at a wavelength capable of sensitizing the biological agent or cell; and activating the light sensitive biological agent or cell.

In certain embodiments of the method, the excitation light for the UCNP comprises a wavelength in the range of about 700 nm to about 1,100 nm (e.g., about 800 nm to about 1,100 nm, about 900 nm to about 1,100 nm, about 1,000 nm to about 1,100 nm, about 700 nm to about 1,000 nm, about 700 nm to about 900 nm, about 700 nm to about 800 nm, about 800 nm to about 1,000 nm), and the emission luminescence of the UCNP comprises a wavelength in the range of about 380 nm to about 700 nm (e.g., about 450 nm to about 700 nm, about 500 nm to about 700 nm, about 550 nm to about 700 nm, about 600 nm to about 700 nm, about 380 nm to about 650 nm, about 380 nm to about 600 nm, about 380 nm to about 550 nm, about 380 nm to about 500 nm). In certain embodiments of the method, the excitation light for the UCNP comprises a wavelength in the range of about 800 nm to about 1,000 nm, and the emission luminescence of the UCNP comprises a wavelength in the range of about 380 nm to about 500 nm.

In certain embodiments of the method, the light sensitive biological agent or cell is a herein disclosed T cell.

In yet another aspect, the invention generally relates to a method for treating cancer in a subject. The method comprises: administering to the subject in need thereof a pharmaceutical composition comprising an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for treating cancer in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for providing anti-tumor immunity in a subject. The method comprises: administering to the subject in need thereof a pharmaceutical composition comprising an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for providing anti-tumor immunity in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for stimulating a T cell-mediated immune response to a cell population or tissue in a subject. The method comprises: administering to the subject in need thereof a pharmaceutical composition comprising an anti-tumor effective amount of a population of T cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for stimulating a T cell-mediated immune response to a cell population or tissue in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for generating a persisting population of genetically engineered T cells in a subject. The method comprises: administering to the subject in need thereof a pharmaceutical composition comprising an anti-tumor effective amount of a population of T cells disclosed herein, and inducing fusion of the first part of the optogenetic dimerizer pair and the second part of the optogenetic dimerizer pair to form a fusion dimer, thereby forming a CAR by joining Component I and Component II.

In yet another aspect, the invention generally relates to a method for generating a persisting population of genetically engineered T cells in a subject. The method comprises: administering to the subject in need thereof a pharmaceutical composition comprising a UCNP disclosed herein and an anti-tumor effective amount of a population of cells disclosed herein, and inducing fusion of the first part of the optogenetic dimerizer pair and the second part of the optogenetic dimerizer pair to form a fusion dimer, thereby forming a CAR by joining Component I and Component II.

For a herein disclosed method, the referred to tumor or cancer may be a liquid tumor or cancer (e.g., selected from the group consisting of: leukemia and lymphoma or a solid tumor or cancer (e.g., selected from the group consisting of: glioma, astrocytoma, skin cancer, breast cancer, prostate cancer, colorectal cancer, renal cancer, stomach cancer, bladder cancer, pancreatic cancer, hepatocarcinoma, lung cancer, endometrial cancer and thyroid cancer).

For a herein disclosed method, inducing fusion of the first part of the optogenetic dimerizer pair and the second part of the optogenetic dimerizer pair to form a fusion dimer comprises applying a light beam comprising a wavelength in the range of about 400 nm to about 500 nm.

For a herein disclosed method, inducing fusion of the first part of the optogenetic dimerizer pair and the second part of the optogenetic dimerizer pair to form a fusion dimer comprises applying a light beam comprising a wavelength in the range of about 600 nm to about 1,100 nm.

### General Discussions

### Extracellular Domain

The target-specific binding element is referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. Examples of cell surface markers that may act as ligands for the antigen binding domain in the CAR include those associated with viral, bacterial and parasitic infections, autoimmune disease and cancer cells.

In one embodiment, the CAR can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens include, for example, a glioma-associated antigen, carcinoembryonic antigen (CEA), beta-human chorionic gonadotropin, alphafetoprotein (AFP), CD70, the leukocyte Ig-like receptor subfamily B (e.g., LILRB4 (John, et al. Molecular Therapy. 26(10):2487-2495), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and CD33. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

In one embodiment, the antigen binding domain portion of the CAR targets an antigen that includes but is not limited to CD19, CD20, CD22, ROR1, CD33, c-Met, PSMA, Glycolipid F77, EGFRvIII, GD-2, MY-ESO-1 TCR, MAGE A3 TCR, and the like.

### Transmembrane Domain

With respect to the transmembrane domain, the CAR comprises one or more transmembrane domains fused to the extracellular antigen binding domain of the CAR. The transmembrane domain may be derived either from a natural or from a synthetic source. Where the source is natural, the domain may be derived from any membrane-bound or transmembrane protein.

Transmembrane regions of particular use in the CARs described herein may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, mesothelin, CD33, CD37, CD64, CD80, CD86, CD134, CD137, CD154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain that naturally is associated with one of the domains in the CAR is used in addition to the transmembrane domains described herein.

### Spacer Domain

In the CAR, a spacer domain, also termed hinge domain, can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

### Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. The term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability. It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARs disclosed herein include those derived from TCR zeta (CD3 Zeta), FcR gamma, FcR beta, CD3 gamma, CD3 delta, CD3 epsilon, CD5, CD22, CD79a, CD79b, and CD66d.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

### Antibodies and Antigen Binding Fragments

One embodiment further provides a CAR, a T cell expressing a CAR, an antibody, or antigen binding domain or portion thereof, which specifically binds to one or more of the antigens disclosed herein. As used herein, a “T cell expressing a CAR,” or a “CAR T cell” means a T cell expressing a CAR, and has antigen specificity determined by, for example, the antibody-derived targeting domain of the CAR.

As used herein, an “antigen binding domain” can include an antibody and antigen binding fragments thereof. The term “antibody” is used herein in the broadest sense and encompasses various antibody structures, including but not limited to monoclonal antibodies, polyclonal antibodies, multi-specific antibodies (e.g., bispecific antibodies), and antigen binding fragments thereof, so long as they exhibit the desired antigen-binding activity. Non-limiting examples of antibodies include, for example, intact immunoglobulins and variants and fragments thereof known in the art that retain binding affinity for the antigen.

A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. In some examples, a monoclonal antibody is an antibody produced by a single clone of B lymphocytes or by a cell into which nucleic acid encoding the light and heavy variable regions of the antibody of a single antibody (or an antigen binding fragment thereof) have been transfected, or a progeny thereof. In some examples monoclonal antibodies are isolated from a subject. Monoclonal antibodies can have conservative amino acid substitutions which have substantially no effect on antigen binding or other immunoglobulin functions. Exemplary methods of production of monoclonal antibodies are known, for example, see Harlow & Lane, Antibodies, A Laboratory Manual, 2nd ed. Cold Spring Harbor Publications, New York (2013).

An “antigen binding fragment” is a portion of a full-length antibody that retains the ability to specifically recognize the cognate antigen, as well as various combinations of such portions. Non-limiting examples of antigen binding fragments include Fv, Fab, Fab′, Fab′-SH, F(ab′)2; diabodies; linear antibodies; single-chain antibody molecules (e.g. ScFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

A single-chain antibody (ScFv) is a genetically engineered molecule containing the VH and VL domains of one or more antibody(ies) linked by a suitable polypeptide linker as a genetically fused single chain molecule (see, for example, Bird et al., Science, 242:423 426, 1988; Huston et al., Proc. Natl. Acad. Sci., 85:5879 5883, 1988; Ahmad et al., Clin. Dev. Immunol., 2012, doi:10.1155/2012/980250; Marbry, IDrugs, 13:543-549, 2010). The intramolecular orientation of the VH-domain and the VL-domain in a ScFv, is typically not decisive for ScFvs. Thus, ScFvs with both possible arrangements (VH-domain-linker domain-VL-domain; VL-domain-linker domain-VH-domain) may be used.

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, Antibodies, A Laboratory Manual, 2nd ed. Cold Spring Harbor Publications, New York (2013); Hilyard et al., Protein Engineering: A practical approach (IRL Press 1992); Borrabeck, Antibody Engineering, 2d ed. (Oxford University Press 1995); each of which is incorporated herein by reference).

### Nucleotides, Vectors and Host Cells

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an embodiment of the invention, the nucleic acid may comprise a codon-modified nucleotide sequence that encodes the antigen binding domain of the inventive CAR. In another embodiment of the invention, the nucleic acid may comprise a codon-modified nucleotide sequence that encodes any of the CARs described herein (including functional portions and functional variants thereof).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., Molecular Cloning: A Laboratory Manual, 3rd ed., Cold Spring Harbor Press, Cold Spring Harbor, N.Y. 2001. The nucleic acids can be constructed based on chemical synthesis and/or enzymatic ligation reactions using procedures known in the art. See, for example, Sambrook et al., Molecular Cloning: A Laboratory Manual, 3rd ed., Cold Spring Harbor Press, Cold Spring Harbor, N.Y. 2001, and Ausubel et al., Current Protocols in Molecular Biology, Greene Publishing Associates and John Wiley & Sons, NY, 1994.

For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs. It is generally appreciated that conditions can be rendered more stringent by the addition of increasing amounts of formamide.

Also provided is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of a nucleic acid described herein.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses.

A number of transfection techniques are generally known in the art (see, e.g., Graham et al., Virology, 52: 456-467 (1973); Sambrook et al., Molecular Cloning: A Laboratory Manual, 3rd ed., Cold Spring Harbor Press, Cold Spring Harbor, N.Y. 2001; Davis et al., Basic Methods in Molecular Biology, Elsevier (1986); and Chu et al, Gene, 13: 97 (1981).

Transfection methods include calcium phosphate co-precipitation (see, e.g., Graham et al., Virology, 52: 456-467 (1973)), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al, Nature, 327: 70-73 (1987)).

In an embodiment, the recombinant expression vectors can be prepared using standard recombinant DNA techniques described in, for example, Sambrook et al., Molecular Cloning: A Laboratory Manual, 3rd ed., Cold Spring Harbor Press, Cold Spring Harbor, N.Y. 2001, and Ausubel et al., Current Protocols in Molecular Biology, Greene Publishing Associates and John Wiley & Sons, NY, 1994. Constructs of expression vectors, which are circular or linear, can be prepared to contain a replication system functional in a prokaryotic or eukaryotic host cell. Replication systems can be derived, e.g., from ColEl, 2μ, plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

### Pharmaceutical Compositions

Biopharmaceutical or biologics compositions (herein, “pharmaceutical compositions”) are provided herein for use in immunotherapy and/or cell therapy in a carrier (such as a pharmaceutically acceptable carrier). The compositions can be prepared in unit dosage forms for administration to a subject. The amount and timing of administration are at the discretion of the treating clinician to achieve the desired outcome. The pharmaceutical compositions can be formulated for systemic (such as intravenous) or local (such as intra-tumor) administration. The pharmaceutical compositions can be formulated for parenteral administration, such as intravenous administration.

The pharmaceutical compositions can be formulated to include a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents, adjuvant agents, and the like, for example, sodium acetate, sodium chloride, potassium chloride, calcium chloride, sodium lactate and the like.

The concentration of active agent(s) in a pharmaceutical composition can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

The following examples are meant to be illustrative of the practice of the invention and not limiting in any way.

### EXAMPLES

Exemplified herein is the successful design of photoswitchable CARs to deliver dual input (antigen+photon)-gated immune response using engineered therapeutic T cells. LiCAR enables strictly light control over the therapeutic activity of CAR T-cells by simply varying the duration of light illumination in vitro. Furthermore, in conjugation with injectable and imaging guided post-treatment removable upconversion nanoplates, LiCAR permits the time- and location-specific CAR T cell-mediated antitumor activity via deep tissue penetrable MR light in vivo. Because this strategy allows for the precise spatiotemporal regulation of the T cell-mediated immune response, we anticipate that it can ultimately lead to the development of new generations of personalized optogenetic immunotherapy, where the timing, location, and dosage of T cell-mediated therapeutic activity can be tailored to the patients' needs.

### Design and Optimization of LiCARs

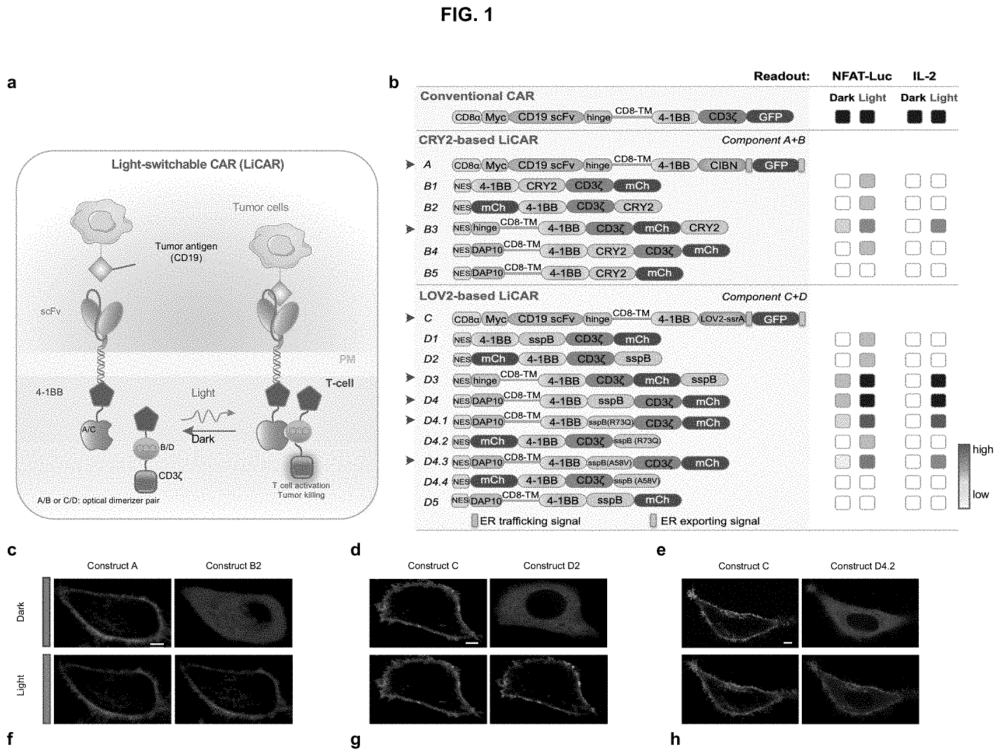

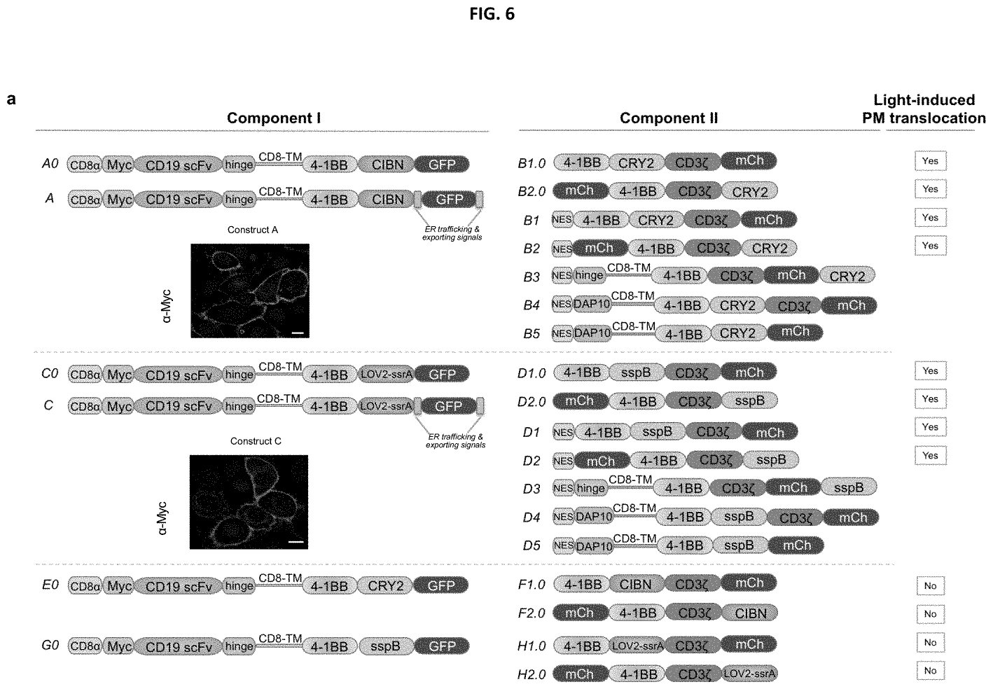

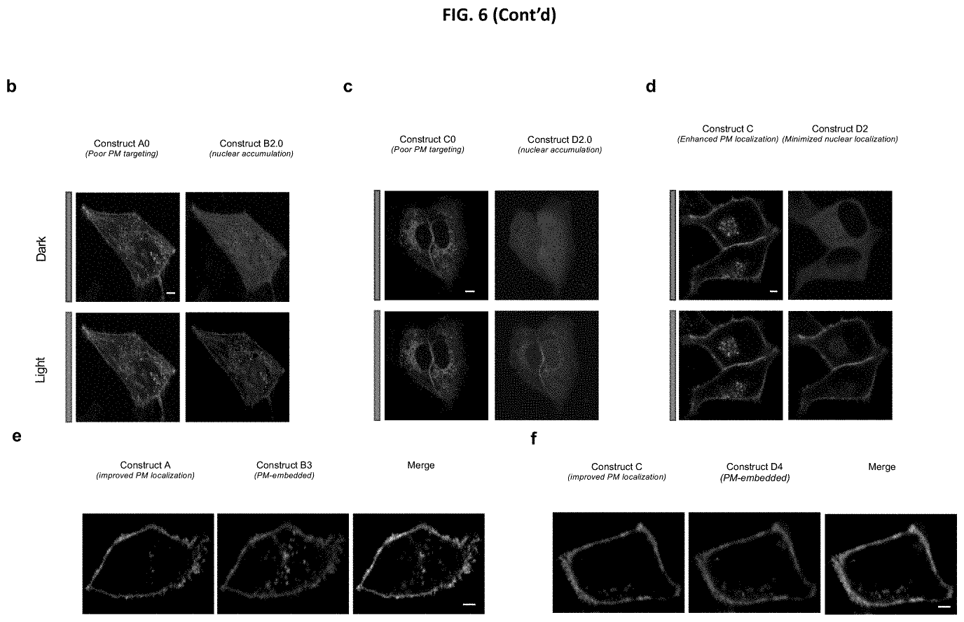

In our envisioned design, LiCARs were engineered by intracellularly splitting functional domains of CAR and installing photo-responsive modules into each half of a split CAR (FIG. 1a-b and FIG. 6a). T cell activation only occurs after the functional assembly of two components of the split CAR in the presence of blue light, thus conferring strict on-off spatiotemporal control over the anti-tumor immune response (FIG. 1a). To test our idea, we set out to design a series of candidate receptors for light-dependent assembly of functional CARs (A+B or C+D combinations; FIG. 1a-b and FIG. 6a). Two optical dimerizers with different activation and deactivation kinetics were utilized. The first pair was composed of the photolyase homology domain of Arabidopsis thaliana cryptochrome 2 (CRY2) and the N-terminal region of its photo-sensitive binding partner CIB1 (the CRY2/CIBN pair). The second system was termed iLID, in which a bacterial peptide ssrA was fused to the C-terminus of Avena sativa light-oxygen-voltage domain 2 (LOV2) and exhibited light-dependent interaction with its binding partner sspB (the LOV2-ssrA/sspB pair). Both optogenetic systems are operated by using flavin that is abundantly present in mammalian cells thus obviating the need to supply exogenous cofactors. (Kennedy 2010 Nat Methods 7, 973-975; Guntas, et al. 2015 Proc Natl Acad Sci USA 112, 112-117.)

Based on these elementary building blocks, we generated a series of hybrid constructs by fusing optical dimerizers with either the extracellular antigen-binding modules or the intracellular signal transduction modules from a conventional CAR (FIG. 6a). In a typical design, Component I of LiCAR shares several similar features with CAR, which bears a signal peptide from T cell surface glycoprotein CD8 alpha chain, a Myc tag to aid extracellular staining, an anti-CD19 scFv, CD8a hinge, the CD8 transmembrane domain, the co-stimulatory 4-1BB domain, and one part of the optical dimerizer pair. GFP was tagged to the C-terminus to aid visualization and detection of the protein expression. The cell surface expression and membrane topology of Component I was confirmed by the non-permeabilized staining of the transfected cells with an anti-Myc antibody (inset, FIG. 6a). The prototypical Component II construct comprises a co-stimulatory 4-1BB domain, the T cell activating ITAM fragment of CD3 subunit, and the complementary part of the optical dimerizer pair, as well as the mCherry (mCh) tag to aid in the visualization and flow cytometry analysis of protein expression.

By using HeLa cells as a model cellular system, we first examined the subcellular distribution of the designed constructs. We found that the first-generation Component I showed poor trafficking toward the plasma membrane (PM), whereas Component II tended to accumulate within the nuclei (constructs A0+B2.0 or C0+D2.0; FIG. 6b-c). To overcome these issues, we inserted ER trafficking and export signal peptides, derived from an inward rectifier potassium channel, into Component Ito enhance PM targeting (constructs A and C). (Ma, et al. 2001 Science 291, 316-319; Gradinaru, et al. 2010 Cell 141, 154-165.) In parallel, we appended a nuclear export signal (NES) from a cyclical AMP-dependent protein kinase inhibitor (PKIa) into Component II in order to minimize its nuclear entry (FIG. 1B-d, and FIG. 6d-f). (Wen, et al. 1995 Cell 82, 463-473.) When co-expressed in mammalian cells, in response to blue light illumination, these improved constructs (A+B2 or C+D2) exhibited light-dependent heteromerization, as reflected by the rapid cytosol-to-PM translocation of Component II (FIG. 1c-e). This process can be repeated multiple times by toggling the light switch (FIG. 1f-h). While the dissociation of the LOV2-based LiCAR complex occurred with a relatively fast half-life (t_{1/2, off}=33-37 s), the reversion of CRY2-CIBN1 binding took a much longer time (t_{1/2, off}=4-5 min)(FIG. 1f-h). The varying range of deactivation half-lives makes it possible to temporally control the duration of the elicited anti-tumor immune response.

### LiCAR Allows for Photo-Tunable and Antigen-Specific Activation of Engineered T Cells

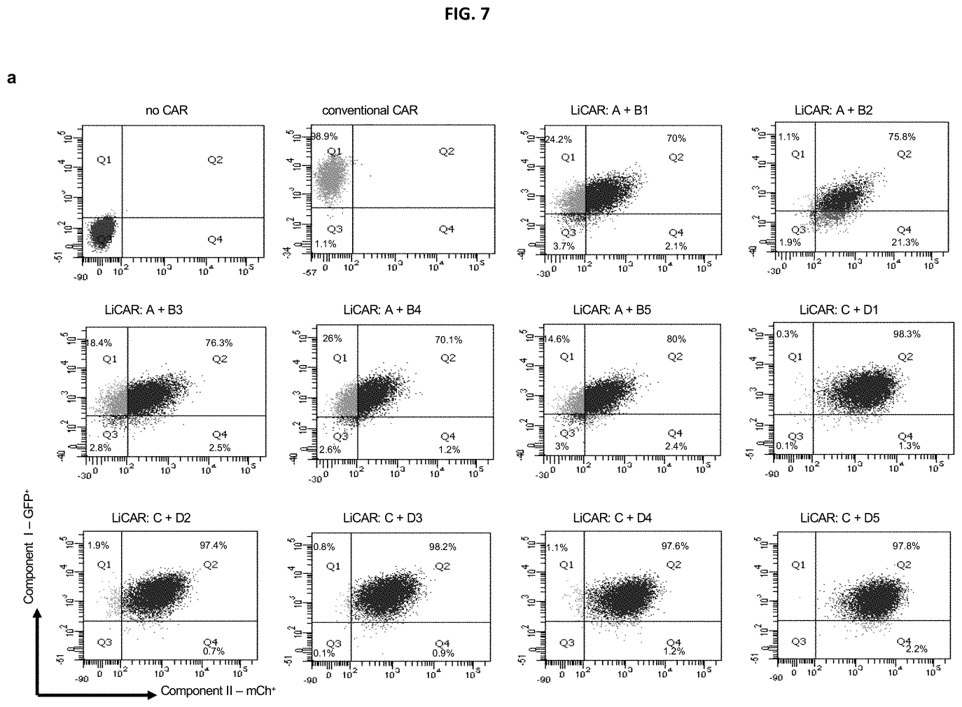

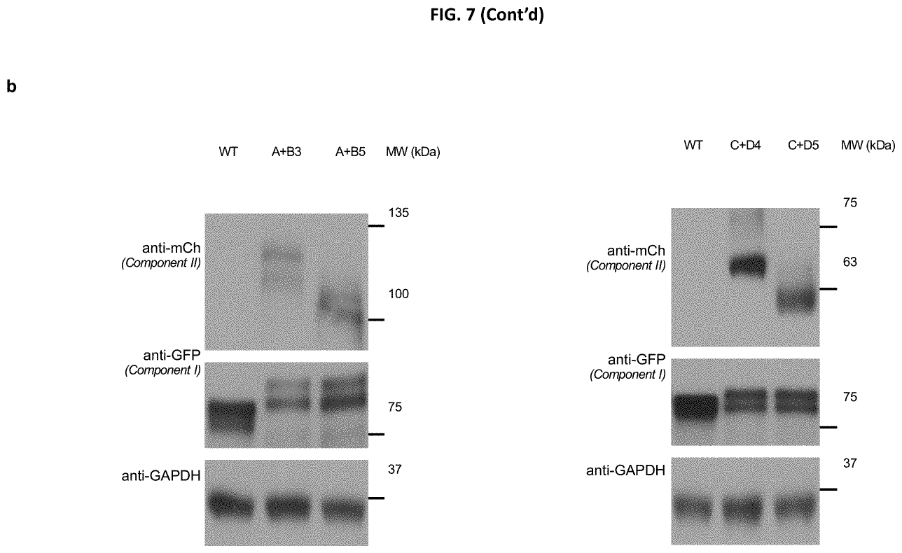

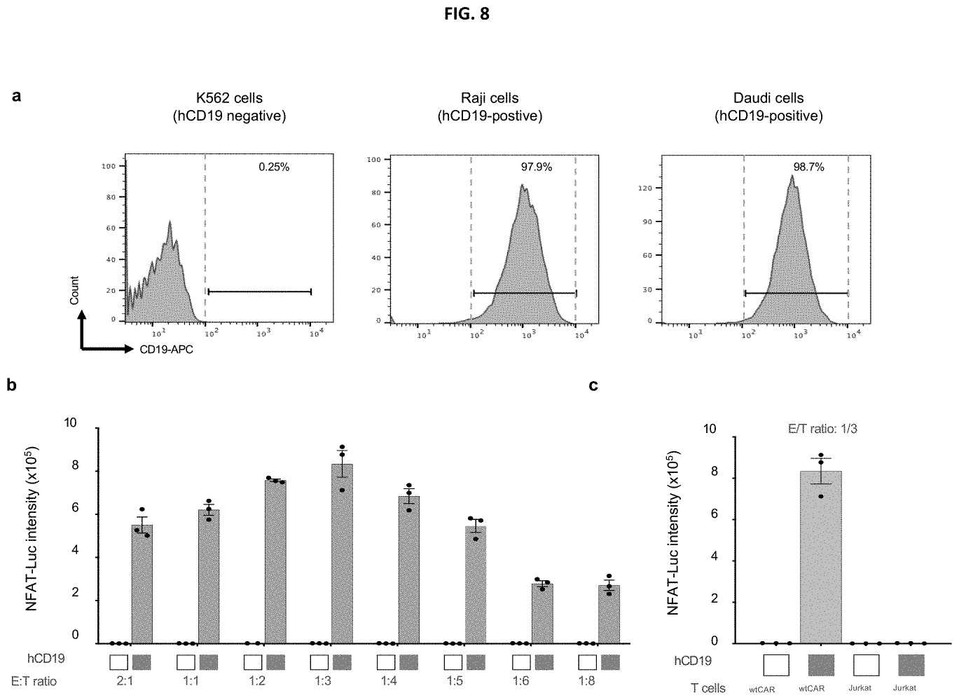

We next sought to screen combinations that would permit the light-induced functional assembly of LiCARs in T cells. We used the NFAT-dependent luciferase (NFAT-Luc) reporter assay as a convenient method to assess the degree of T cell activation. (He, et al. 2015 Elife 4.) In human Jurkat-Luc T cells co-transduced with retroviruses encoding the split CAR components, we were able to achieve a transduction efficiency of 70-80% for the CRY2-based LiCAR (A+B) and over 97% for the LOV2-based LiCAR (C+D; GFP as indicator for Component I and mCherry for Component II; FIG. 7a). The co-expression of both components at the protein level in T cells was further confirmed by immunoblotting (FIG. 7b). NFAT-Luc Jurkat T cells expressing engineered receptors were then engaged with human CD19 (hCD19)-negative K562 leukemia cells or hCD19-positive Raji lymphoma cells (FIG. 8a-c). In the dual presence of cognate tumor antigen (hCD19⁺ Raji lymphoma cells) and light stimulation, we detected a pronounced increase in NFAT-driven reporter bioluminescence, indicating the antigen/light-dependent activation of engineered T cells (A+B1, A+B2, C+D1, or C+D2; FIG. 1i). However, these prototypical LiCAR T-cells did not appear to be efficiently activated, as they only produced 16-21% of the maximal response seen in WT CAR T-cells (FIG. 1i). We reasoned that targeting both components to the PM might reduce the entropic cost of driving the cytosolic Component II to couple with the PM-resident Component I. Therefore, we continued to modify Component II by adding the transmembrane domain of CD8 (Constructs B3 or D3) and/or the homodimeric DAP10 ectodomain (B4 or D4), the latter of which was added to double the available numbers of CD3-ζITAMs in each assembled LiCAR complex in order to amplify the activation signals. (Wu, et al. 1999 Science 285, 730-732; Wu, et al. 2015 Science 350, aab4077; Irving, et al. 1993 J Exp Med 177, 1093-1103.)

We also reordered the positions of intracellular CAR components and optical dimerization modules, anticipating that we would be able to identify the best configuration for maximal light-inducible reconstitution of functional CARs (FIG. 1B and FIG. 6a). Ultimately, we identified the best combinations for both CRY2- and LOV2-based LiCARs, which led to 39% (A+B3) and 93% (C+D4; FIG. 1i) of the maximal response in the NFAT-Luc assay, respectively. As a stringent control, defective LiCAR T-cells (C+D5) lacking CD3ζ-ITAM in Component II did not show discernible antigen/light-dependent changes (right, FIG. 1i).

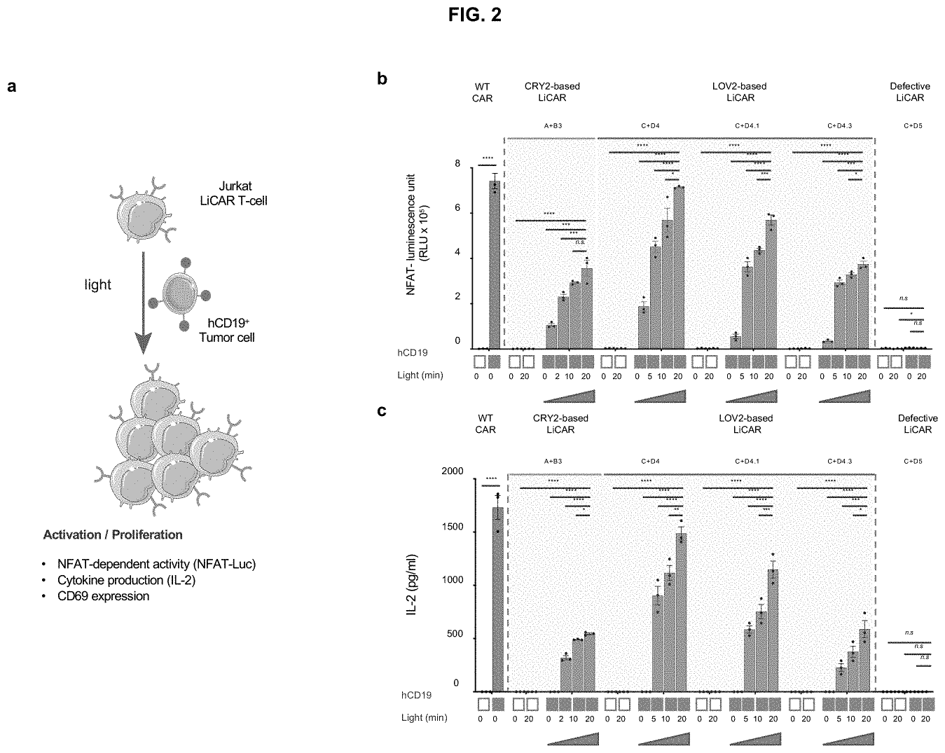

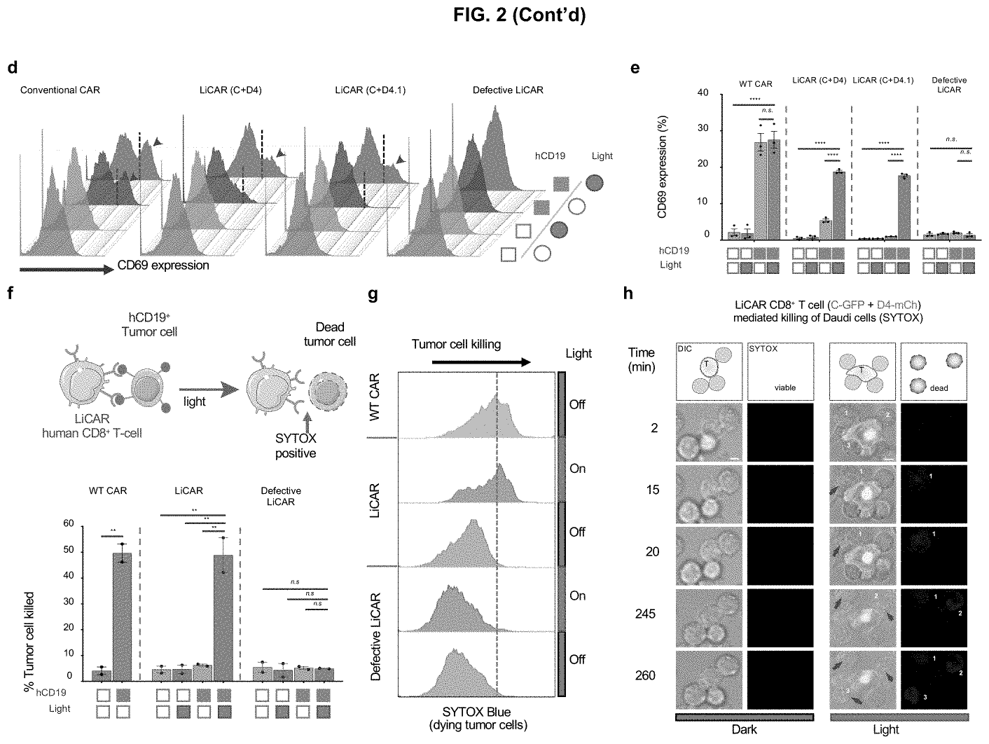

Nevertheless, these improved LiCAR T-cells (A+B3 or C+D4) displayed appreciable dark activity (i.e., pre-activation of NFAT-Luc in the dark; FIGS. 1i and 2a-b). This is probably due to two reasons: (i) the relatively strong binding affinity of iLiD (K_{d}: 4.7±0.7 μM (dark) vs 0.132±0.005 μM (lit)); (ii) the highly sensitive nature of the synthetic NFAT-Luc reporter, which bears multiple copies of NFAT response elements in the promoter. We therefore explored two additional weaker versions of iLID, in which wild type sspB was mutated to sspB-R73Q (Kd: 47±13 μM (dark) vs 0.8±0.1 μM (lit)) (FIG. 1B-i), or sspB-A58V (K_{d}: 56±6 μM (lit)) (FIG. 1b-i). (Guntas, et al. 2015. Proc Natl Acad Sci USA 112, 112-117; Zimmerman, et al. 2016 Biochemistry 55, 5264-5271.) We found that the use of sspB-R73Q and sspB-A58V led to a substantial reduction of background activation, but it also reduced the LiCAR activity to 71% (C+D4.1) and 51% (C+D4.3) of the maximal response in the NFAT-Luc assay, respectively (FIG. 1i). Next, we examined the antigen/light-dependent expression of IL-2—a naturally existing NFAT target gene—as a stringent and more physiologically relevant readout in T cells (FIG. 2c). In both the NFAT-Luc reporter assay and the IL-2 ELISA assay, LiCAR T-cells exhibited light-tunable activation of NFAT-dependent activity (FIG. 2b-c). Most importantly, we did not detect the pre-activation of IL-2 production in the dark for all the combinations of both CRY2- and LOV2-based LiCARs (FIG. 2c). Collectively, these data established the feasibility of using light to fine-tune the dosage of T cell activity.

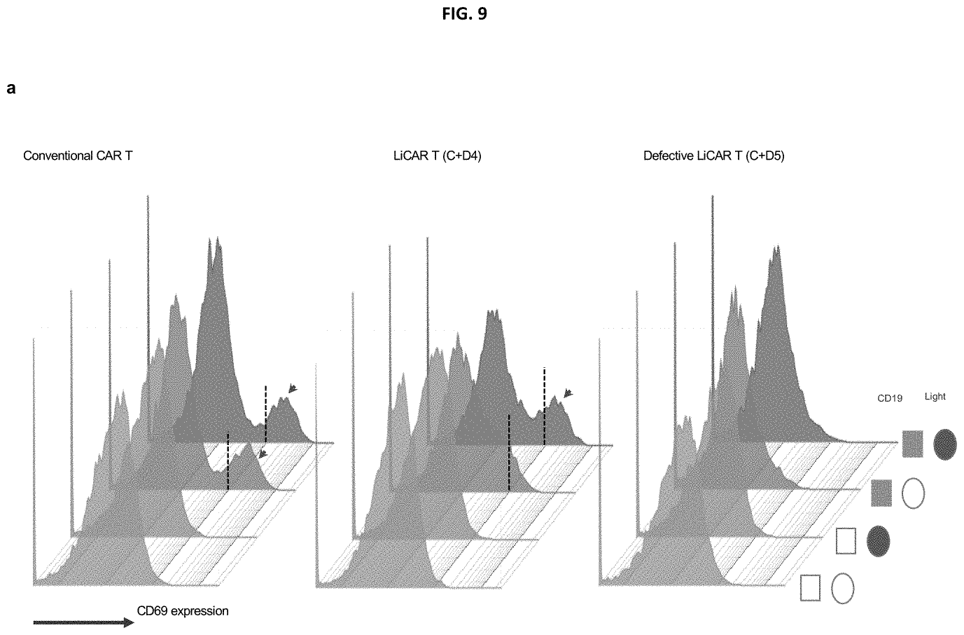

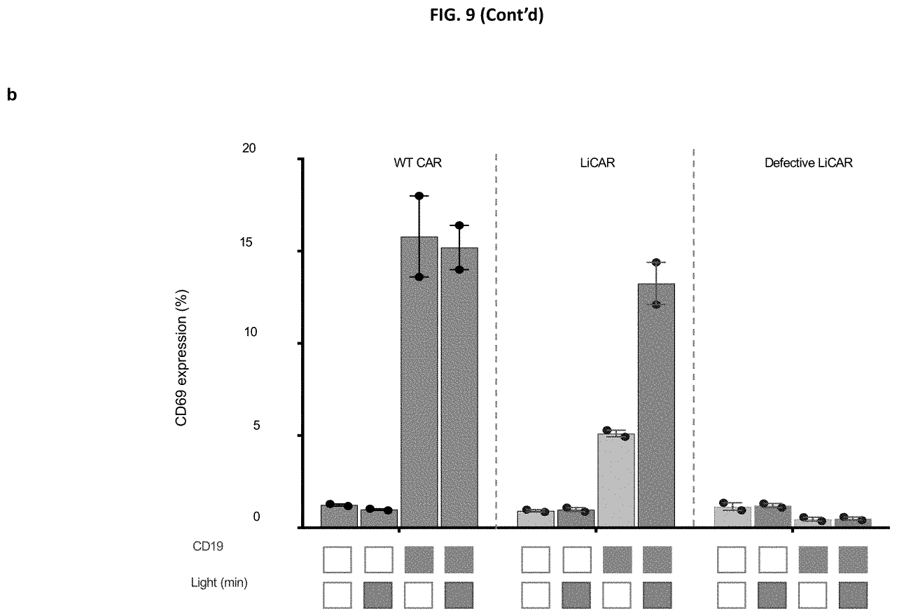

Notably, the LOV2-based LiCAR seemed superior to rival the CRY2-based version by photo-triggering T cell activation to an extent comparable to WT CAR T-cells (FIG. 2b-c). We, therefore, focused on the C+D4 or C+D4.1 combination for further functional characterization. Following T cell activation, we set out to evaluate an early event indicative of T cell activity by monitoring CD69 expression in Jurkat T cells (FIG. 2d-e). CD69 is up-regulated within 2-4 hours after T cell activation and is reported to be essential for lymphocyte retention in lymphoid organs. (Testi, et al. 1989 J Immunol 143, 1123-1128.) We compared the CD69 expression level in engineered Jurkat T cells that were engaged with either hCD19⁻ or hCD19⁺ tumor cells, before and after photo-stimulation. While conventional CAR T-cells only required the cognate hCD19 antigen to activate T cells and boost CD69 expression, engineered T cells expressing LiCAR showed a light-dependent increase in CD69 expression in the presence of hCD19⁺ tumor cells, including Daudi (FIG. 2d) and Raji lymphoma cells (FIG. 9). Again, the C+D4.1 combination using a weaker version of iLID showed a substantially lower background activity in the dark compared to the C+D4 constructs (FIG. 2d-e). As control, CD19⁻K562 cells did not induce any significant change in CD69 expression in either the dark or lit state. Taken together, these results validated the successful construction of LiCAR to achieve dual input-gated control of T cell activation in a photo-tunable manner.

### LiCAR T-Cells Enable Photo-Inducible Killing of Tumor Cells Ex Vivo

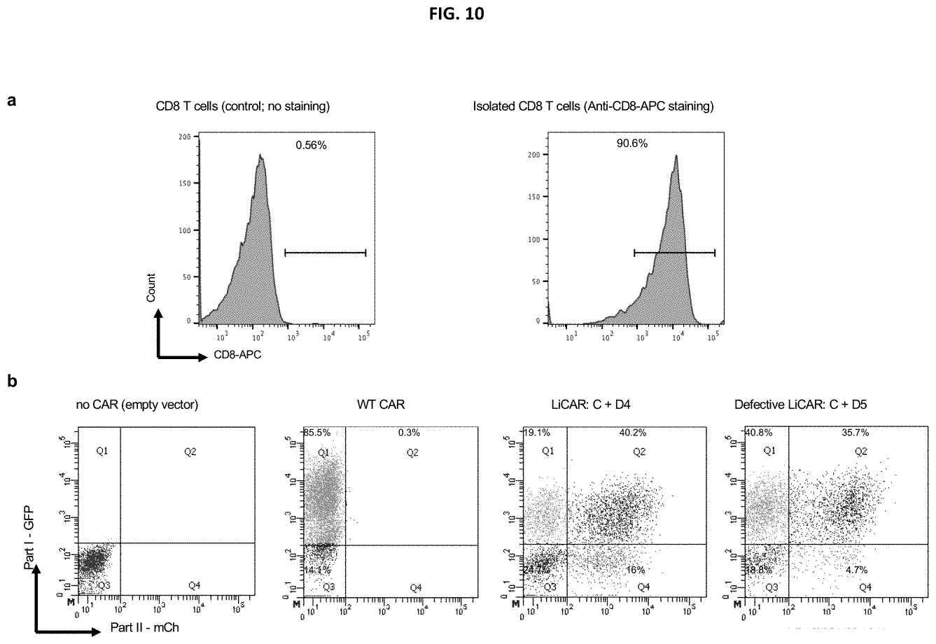

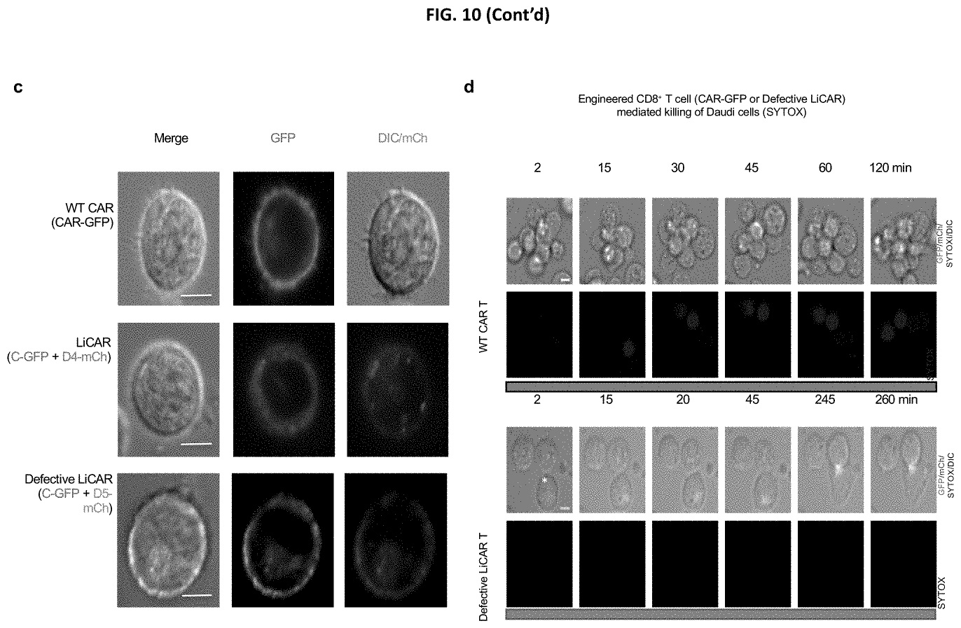

A hallmark representative of CAR T-cells is their ability to induce apoptosis in targeted tumor cells. Controlled activation of CAR T-cells to trigger inducible cytotoxicity against tumors is one of the most desirable features of smart immunotherapy. Hence, we moved on to test the LiCAR's ability to mediate the killing of cognate tumor cells (e.g., Daudi cells derived from Burkitt's lymphoma) when expressed in primary human CD8⁺ T cells (FIG. 2f-h). Both components of LiCAR were co-expressed in CD8⁺ T cells derived from peripheral blood mononuclear cells (PBMC), as confirmed by flow cytometry analysis and confocal imaging (FIG. 10a-c). Subsequently, the cytotoxic activity of engineered CD8⁺ T cells against co-cultured with K562 (hCD19⁻) or Daudi (hCD19⁺) cancer cells, was quantified by staining target cells with the SYTOX Blue dye, which only penetrates dead cells with a compromised plasma membrane. After overnight incubation in the presence of pulsed blue light illumination, we observed a light-dependent boost of tumor cell killing, as reflected in the rightward shift of the SYTOX Blue staining peak in the flow cytometric histograms (FIG. 2g). The degree of targeted tumor cell killing was comparable to that observed with WT CAR T-cells. As a stringent control, the killing of cognate tumor cells was not observed in the defective LiCAR group (FIG. 2f-g). Noncognate tumor cells (hCD19⁻K562 cells) survived throughout the assay, regardless of the presence of light. This attests to the high tumor antigen specificity of the designed LiCAR T-cells.

To better visualize the spatiotemporal features of CD8³⁰ LiCAR T-cells mediated killing in response to light illumination, we performed time-lapse confocal imaging of LiCAR T-cells mixed with hCD19⁺ Daudi cells (FIG. 2h). Engineered human CD8⁺ T cells expressing WT CAR or defective LiCAR were used as positive and negative controls, respectively (FIG. 10d). Again, SYTOX blue dye was added to monitor dying cells with compromised plasma membrane. Within 15 minutes of exposure to blue light, we started to observe the appearance of blue staining in Daudi cells engaged with LiCAR-expressing human CD8⁺ T cells (FIG. 2h), but not in those in the dark group (FIG. 2h). Collectively, LiCAR T-cells were able to inducibly mount anti-tumor cytotoxicity toward cognate target cells in the dual presence of tumor antigen and light ex vivo.

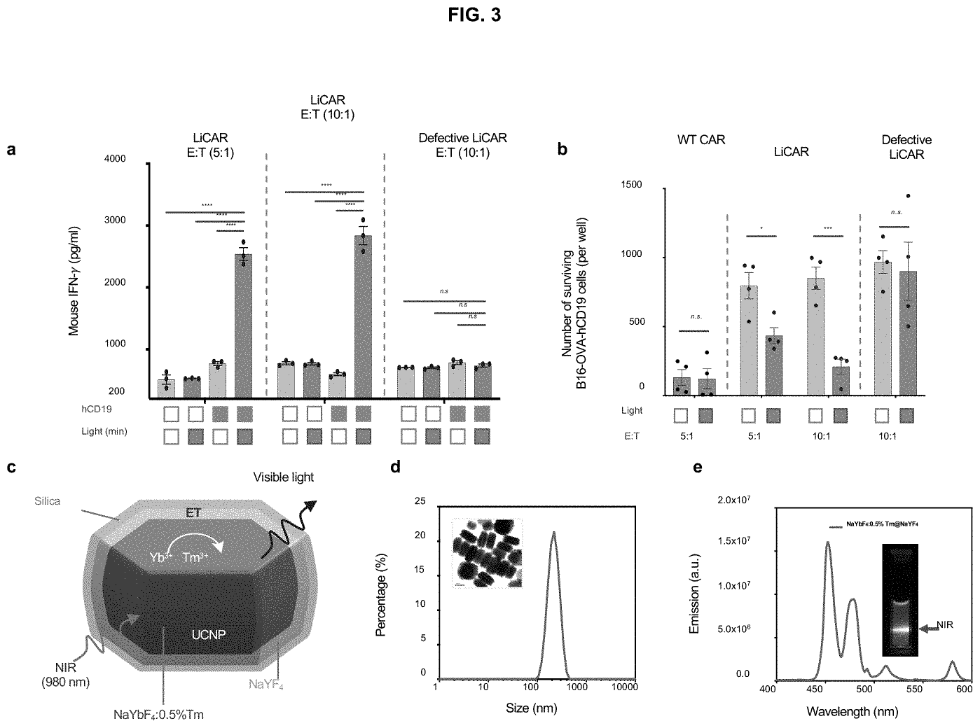

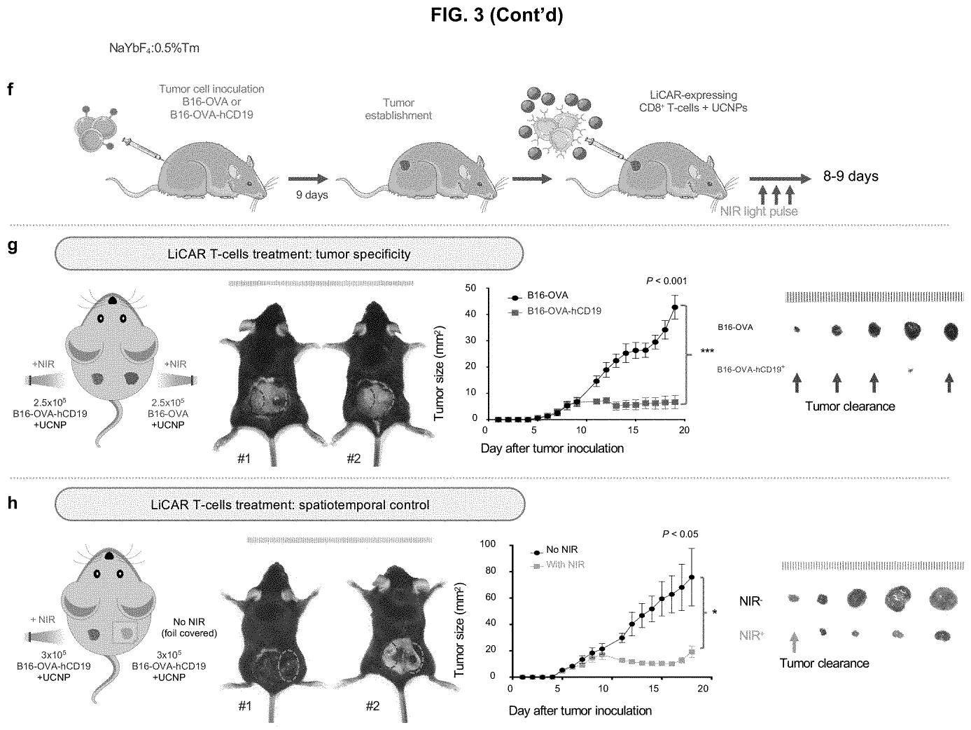

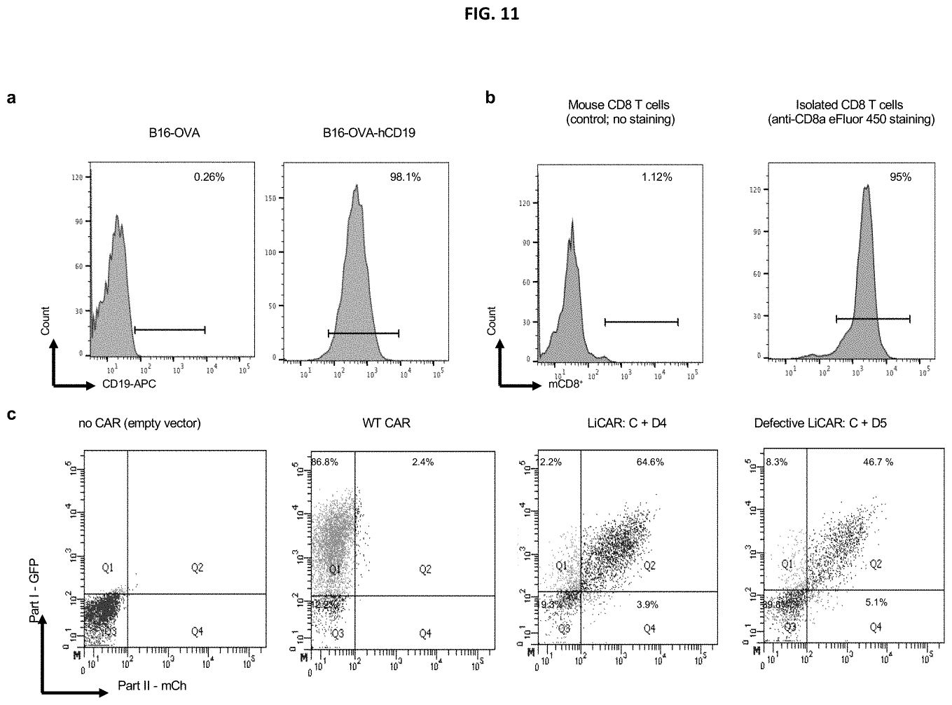

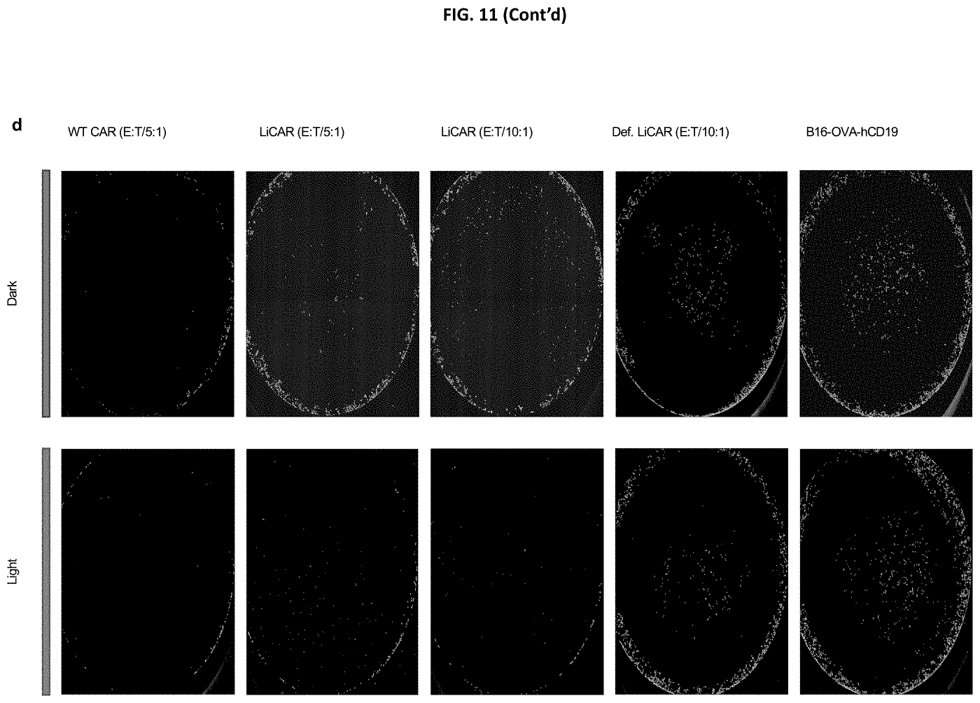

Next, we performed similar ex vivo co-culture experiments to examine whether engineered CD8⁺ T cells derived from mice would likewise trigger productive immune responses and evoke cytotoxicity against cancer cells upon blue light stimulation. To test this, we resorted to a mouse B16-OVA melanoma cell line stably expressing human CD19 (B16-OVA-hCD19) and used the B16-OVA melanoma cells as the CD19-negative control (FIG. 11a). (Chen, et al. 2019 Nature 567, 530-534.) WT CAR, LiCAR, and defective CAR constructs were individually transduced into mouse CD8⁺ T cells with their expression confirmed by flow cytometry (FIG. llb-c). Subsequently, we quantified the production of mouse IFN-γ, which is secreted by activated T cells and plays a critical role in regulating cytotoxic CD8⁺ T cell expansion in response to antigen recognition. (Maraskovsky, et al. 1989 J Immunol 143, 1210-1214.) Indeed, the engineered B16-OVA-hCD19 cells effectively activated LiCAR-expressing mouse CD8 T-cells, as reflected by the production of IFN-γ in the dual presence of CD19 and blue light, as reflected by the production of IFN-γ at both E:T ratio of 5:1 or 10:1 (FIG. 3a). However, there is certain amount of IFN-γ detected in all T cell groups co-cultured with B16-OVA tumor cells or in defective LiCAR T-cells engaged with either B16-OVA or B16-OVA-hCD19 cells (FIG. 3a). This is likely due to the pre-treatment of primary T cells with CD3/CD28 antibodies to make them amenable for viral transduction. LiCAR-expressing CD8⁺ T-cells showed photo-inducible killing of B16-OVA-hCD19 melanoma cells, with an E:T ratio of 10:1 showing the highest potency (FIG. 3b and FIG. 11d). By comparison, WT CAR T-cells induced tumor killing in a light-independent manner (FIG. 3b and FIG. 11d; left); whereas defective LiCAR T-cells co-cultured with either B16-OVA or B16-OVA-hCD19 cells showed no or negligible light-induced effects (FIG. 3b and FIG. 11d; right). Together, we have demonstrated the successful engineering of photoswitchable CAR T-cells from both humans and mice to kill cancer cells derived from hematological malignancies (e.g., lymphoma) and solid tumors (e.g., melanoma).

A hybrid nano-optogenetic platform for NIR light instructed tumor killing in vivo

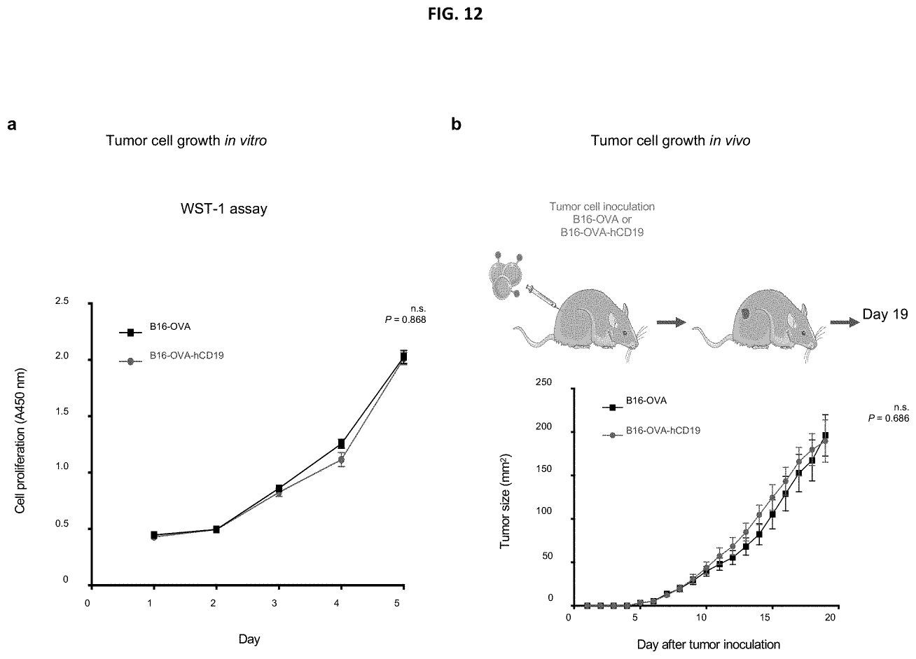

To further validate inducible and selective tumor killing mediated by LiCAR T-cells in vivo, we generated a syngeneic melanoma mouse model of CD19-positive solid tumors by inoculating rodent B16-OVA-hCD19 cells into the flanks of C57BL/6J mice (FIG. 12). (Chen, et al. 2019 Nature 567, 530-534.) Again, B16-OVA melanoma cells without hCD19 were used as negative control. These two cancer cell lines showed comparable growth rates both in vitro (FIG. 12a) and in vivo (FIG. 12b), thus ruling out the possibility that hCD19 per se might induce the differential growth of tumor cells.

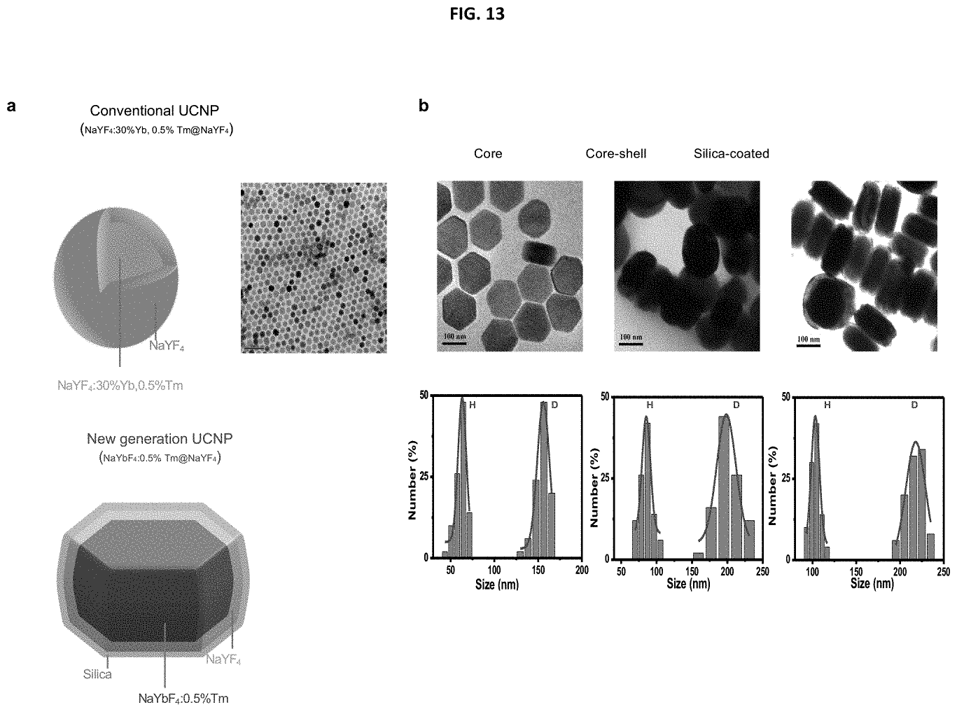

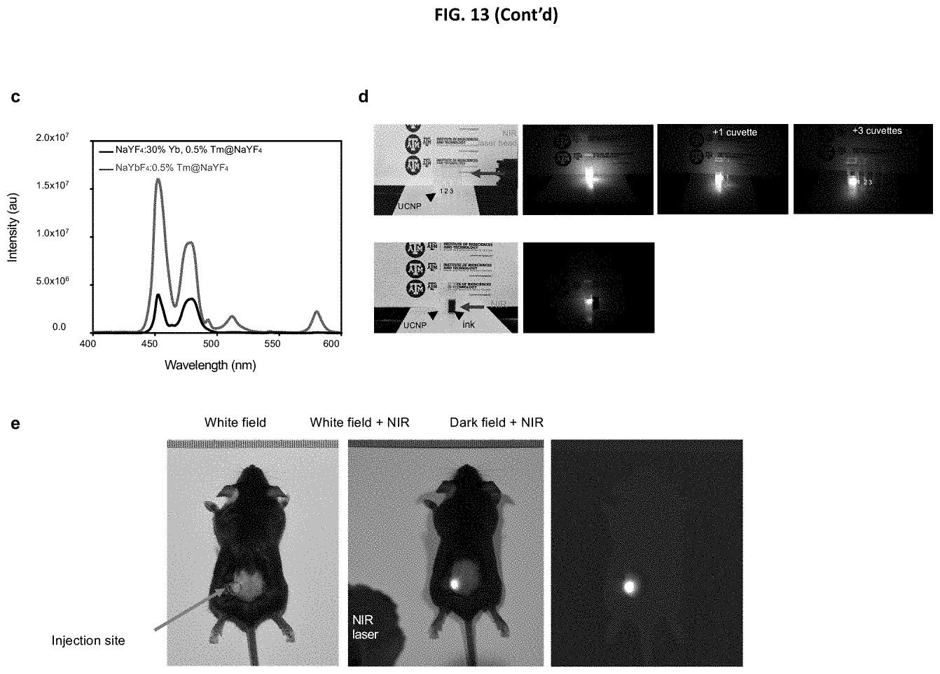

We then set out to combine LiCAR T-cells with MR light-activatable upconversion nanoparticles (UCNPs) to mitigate the tissue penetration issue associated with the in vivo application of blue light-activatable optogenetic tools. (Yu, et al. 2019 Adv Healthc Mater 8, e1801132.) We resorted to lanthanide-doped UCNPs as injectable nanoscale light transducers in order to enable wireless optogenetics, thereby obviating invasive procedures, such as implanting micro-LEDs or inserting fiber optics in tissues to deliver visible light. (Tan, et al. 2017 Trends Biotechnol 35, 215-226.) The most significant advantage of upconversion nanoparticles is their unusual inverse excitation and emission profiles: UCNPs are excited using low power, deep tissue-penetrating, near infrared radiation; but this low energy input can be efficiently converted to a higher energy output emission at diverse shorter wavelengths, including blue light (400-500 nm) that can in vivo activate LiCAR T-cells (FIG. 3c). We have previously employed this strategy to remotely control Ca²⁺ signaling and the immune system during dendritic cell-based immunotherapy. (He, et al. 2015 Elife 4.; Nguyen, et al. 2018 Cell Calcium 75, 79-88.) Here, to enhance upconversion luminescence intensity, we designed injectable hexagonal shaped upconversion nanoplates (UCNPs) with a core-shell composition of β-NaYbF₄:0.5% Tm@NaYF₄, in which Yb³⁺ serves as the sensitizer to accept excitation energy and Tm³⁺ serves as the emitter (FIG. 3c). Compared to the conventional ˜30 nm spherical β-NaYF4:30%Yb,0.5% Tm@NaYF4 core-shell UCNPs used previously, such UCNPs possessed an elevated Yb³⁺ concentration and enlarged nanoparticle size (FIG. 3d and FIG. 13a). (He, et al. 2015 Elife 4.) This nanoplate design is expected to be able to enhance upconversion luminescence. This is due to the prior knowledge that the increased amount of Yb³⁺ could enhance the energy absorption ability of the UCNPs and that surface defect-induced luminescence quench can be further significantly reduced in large nanoparticles. In particular, owing to the smaller ionic size of Yb³⁺ compared to Y³⁺, the replacement of Y³⁺ with Yb³⁺ in UCNPs synthesis suppressed the nucleation process and pronouncedly prolonged nanocrystal growth. (Huang, et al. 20151 J. Mater. Chem. C.) In this regard, the number of nanoparticles in the reaction solution will be less, but their size will be enlarged when using the same amount of lanthanide precursors. As a result, the synthesized β-NaYbF₄:0.5% Tm core possesses a hexagonal plate shape with an enlarged size (˜155 nm in diameter and ˜62 nm in height; FIG. 13b). The produced β-NaYbF4:0.5% Tm@NaYF₄ core-shell nanoplate displayed a hexagonal plate shape with a size of approximately 200 nm×85 nm (FIG. 13b). We observed that the resultant core/shell nanoplates via these two modifications (higher Yb' doping and larger size core/shell structure) indeed markedly boosted the brightness of upconversion luminescence brightness (FIG. 3e and FIG. 13c). When excited at 980 nm, the synthesized UCNPs exhibited intense emission peaks in the blue light range, with an intensity increased by 4.5-fold when compared to the conventional UCNPs with the same amount of total lanthanide ions (FIG. 13c). To further transfer these UCNPs from the organic solvent to an aqueous solution, a layer of silica shell was coated on the surface of the core-shell UCNPs, giving the final silica-coated core-shell nanoplate at a size of ˜217 nm (diameter)×˜103 nm (height) (FIG. 3d and FIG. 13b) or an apparent hydrodynamic radius of approximately 220 nm (FIG. 3d). When illuminated with a 980-nm laser, blue light emitting from the UCNP-containing cuvette in aqueous solutions can easily be seen by the naked eye, even after penetrating through multiple water-containing plastic cuvettes juxta-positioned to the UCNPs (FIG. 13d). When the silica-coated UCNPs were subcutaneously injected into mice, we detected bright blue light emission from the injection site following exposure to a brief pulse of NIR light illumination (FIG. 13e). Together, these data clearly established the feasibility of using our synthesized UCNPs to enable wireless optogenetics in living animals.

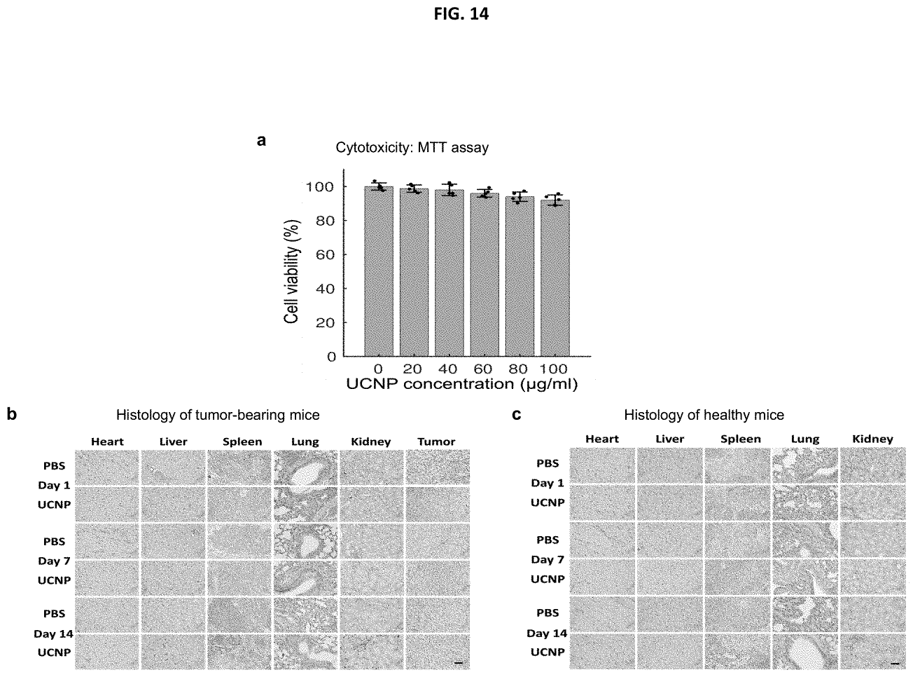

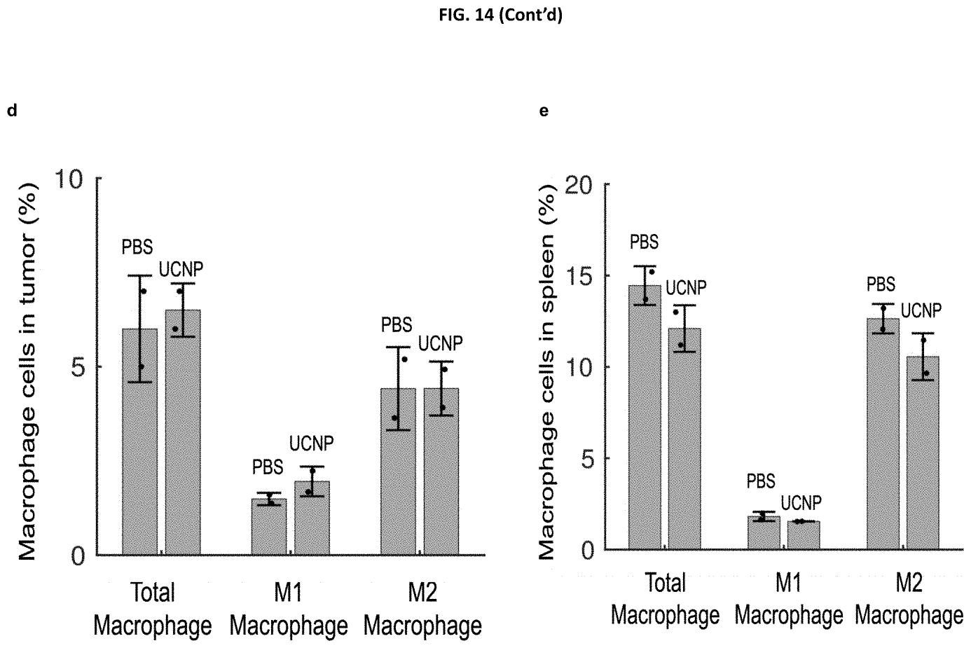

To examine their biocompatibility, we moved on to characterize the potential in vitro cytotoxicity and in vivo biosafety of silica-coated UCNPs. In the cytotoxicity study, B16-OVA-hCD19 cells were incubated with culture media containing UCNPs at increasing concentrations (0 μg/ml-100 μg/ml). Using an MTT assay, we found that the cell viability was insignificantly affected by the UCNPs. With up to 100 μg/ml UCNPs in the cell culture solution, the cell viability remained at more than 90%, thus indicating no overt cytotoxicity of the UCNPs (FIG. 14a). Furthermore, we investigated the in vivo toxicity of UCNPs through histopathological studies and blood sample analyses. In both studies, tumor-bearing mice were injected with UCNPs (1 mg/ml, 150 μL; or an effective concentration of 35 μg/ml within a tumor with a diameter of 2-cm) and sacrificed at different times (1 day, 7 days and 14 days) after UCNP-injection. PBS-injected mice at the tumor sites were used as the control. By comparing H&E stained images of the major organs from UCNP- or PBS-injected mice, we found that neither group displayed noticeable organ damage or inflammatory lesions (FIG. 14b-c), suggesting negligible toxicity of UCNPs to major organs including heart, liver, spleen, lung, and kidney. In the blood analyses, UCNP-injected mice showed similar parameters compared to the PBS-injected group (Table S1), with both falling within the normal range. In particular, the white blood cells of both groups were at the similar level (Table S1), which indicates that no noticeable immune responses were elicited by the UCNPs. In addition to the tumor-bearing mice, the in vivo biosafety studies were also performed on healthy mice, which also showed no obvious toxicity induced by the UCNPs at this dosage (FIG. 14c). Because macrophages are capable of phagocytosing foreign bodies, we further examined whether silica-coated UCNPs could cause macrophage dysfunction by comparing macrophage numbers and polarization in PBS- or UCNP-injected tumor bearing mice at 14 days after injection. We found no significant difference in the total, M1, or M2 subpopulations of macrophages (isolated from spleen or tumor) between the two tested groups (FIG. 14d-e), indicating that UCNPs did not lead to aberrant changes in macrophage function. Together, these data clearly establish the excellent biocompatibility of our synthesized silica-coated UCNPs in living animals.

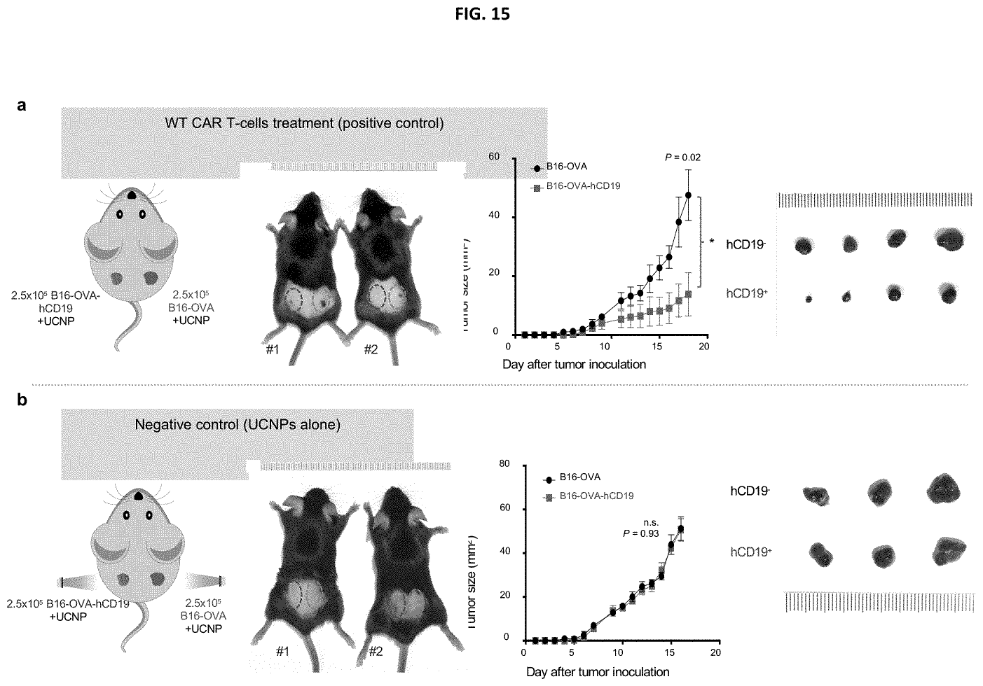

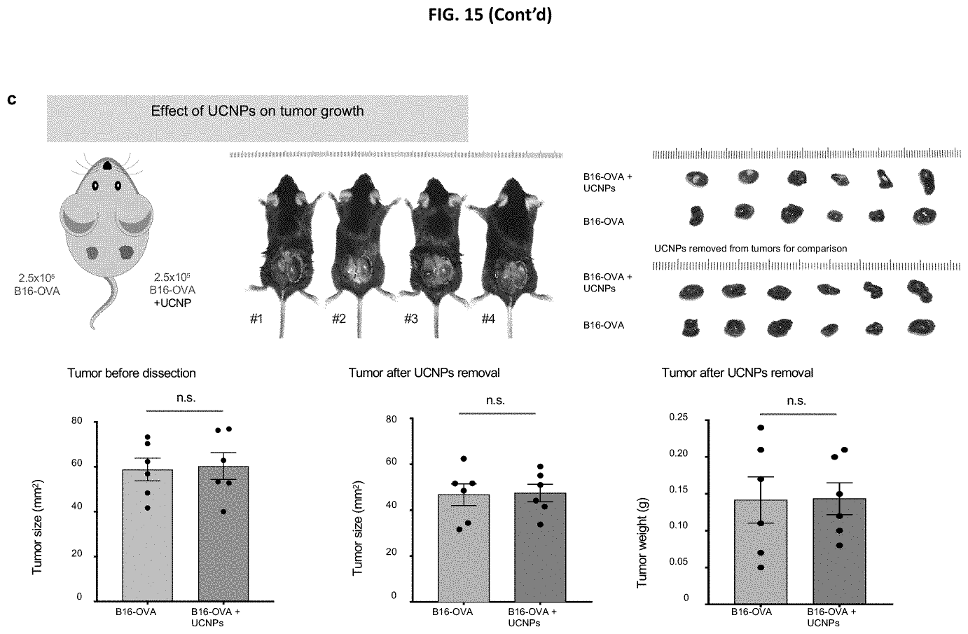

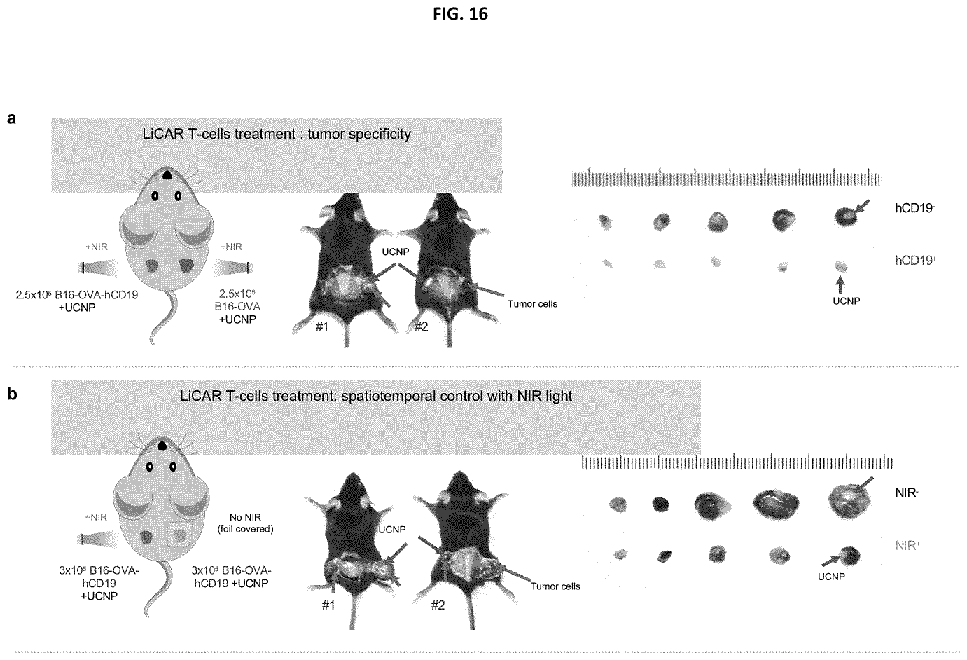

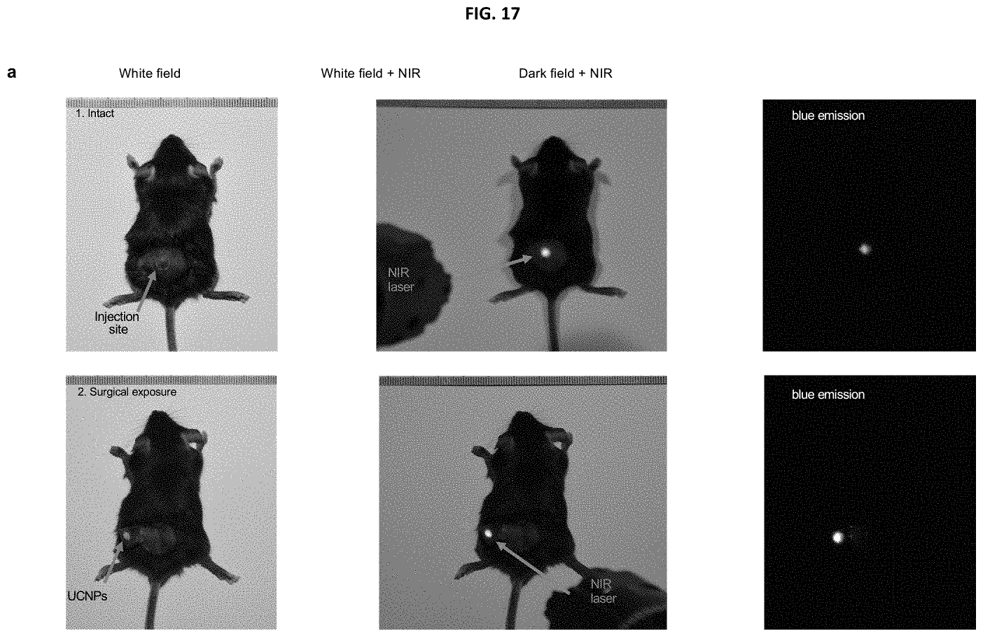

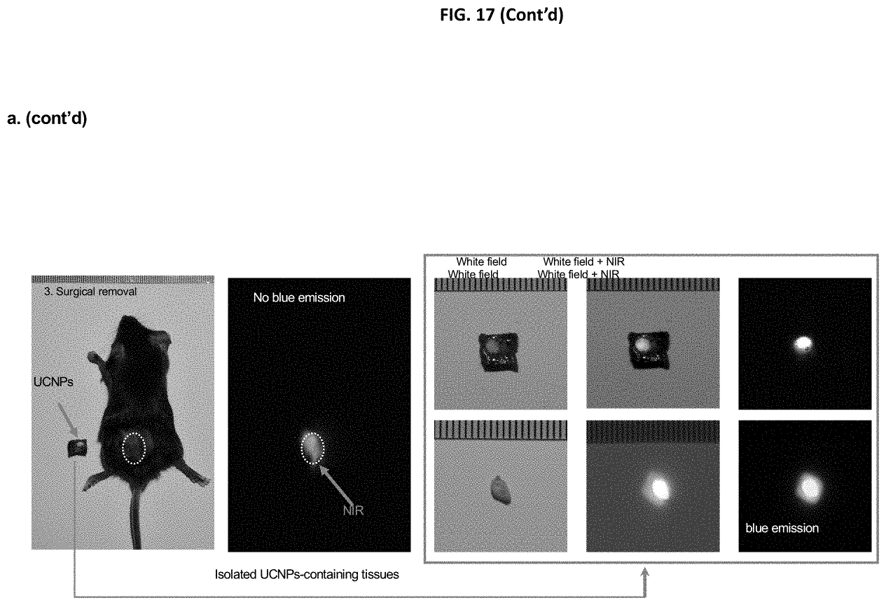

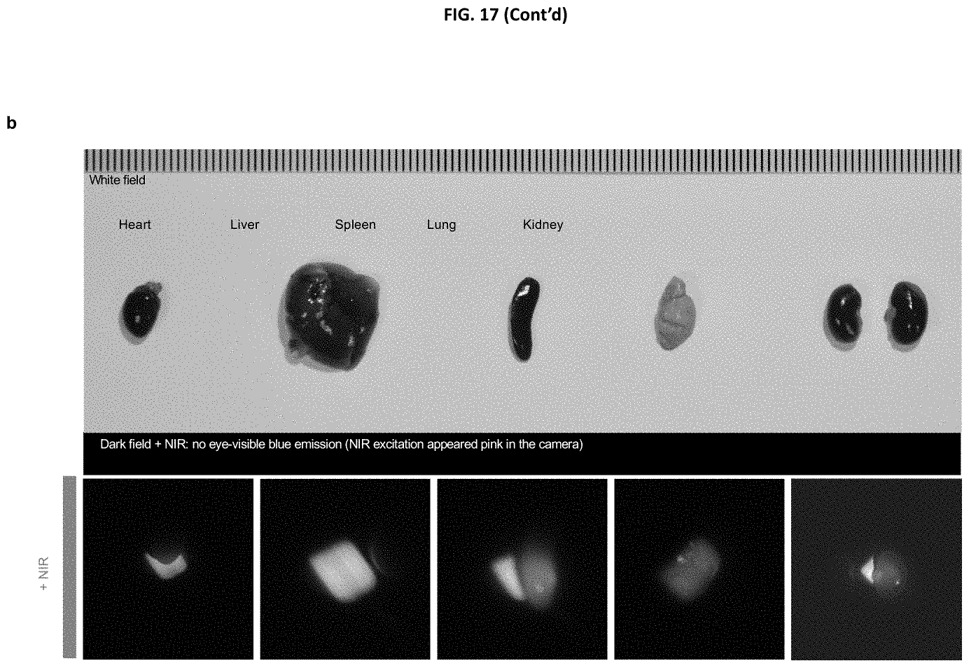

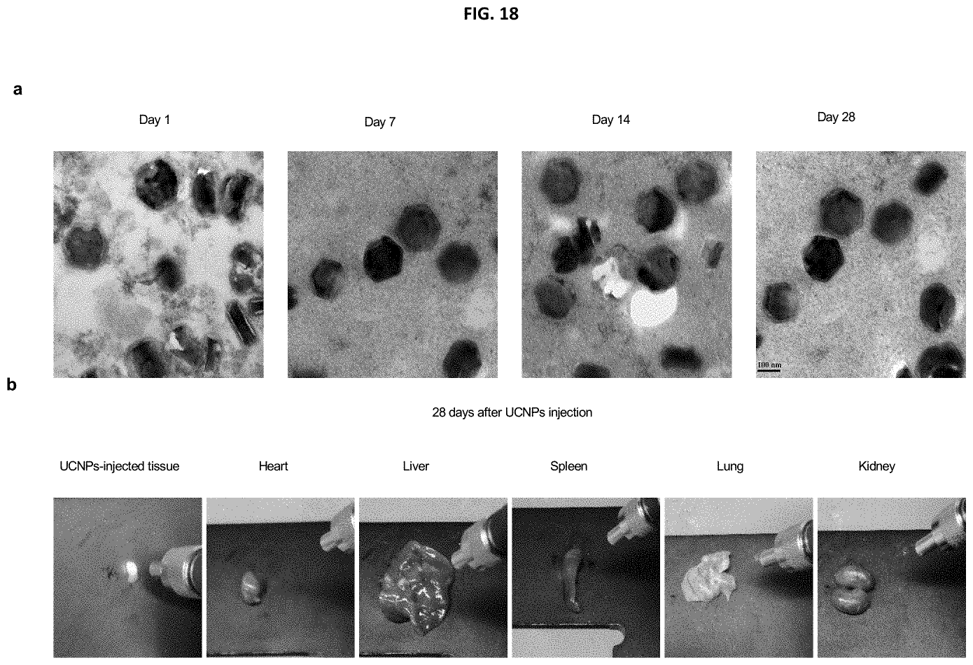

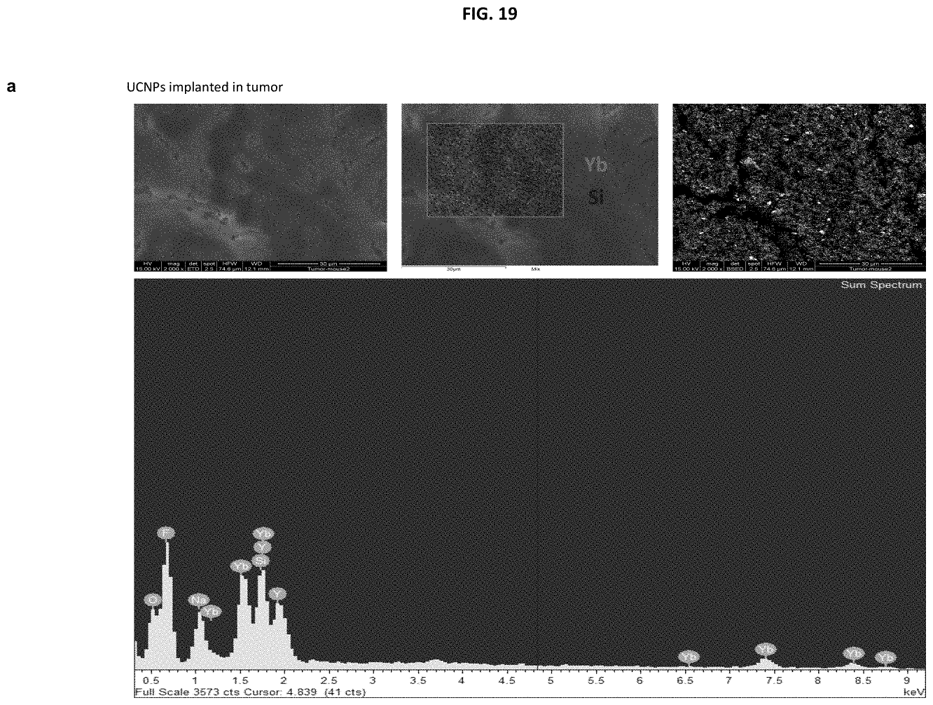

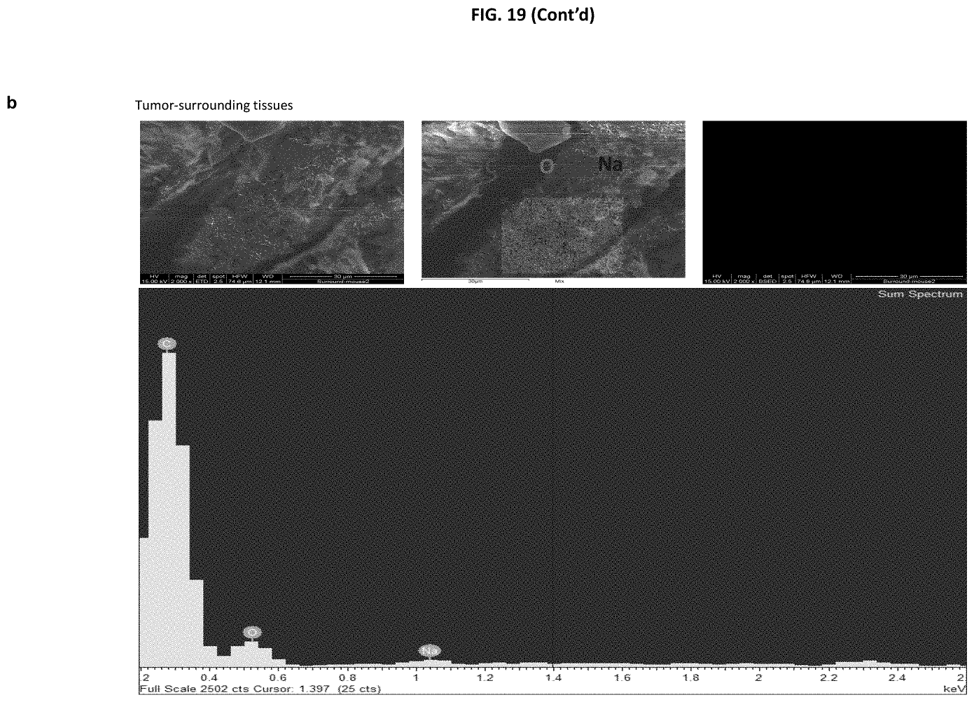

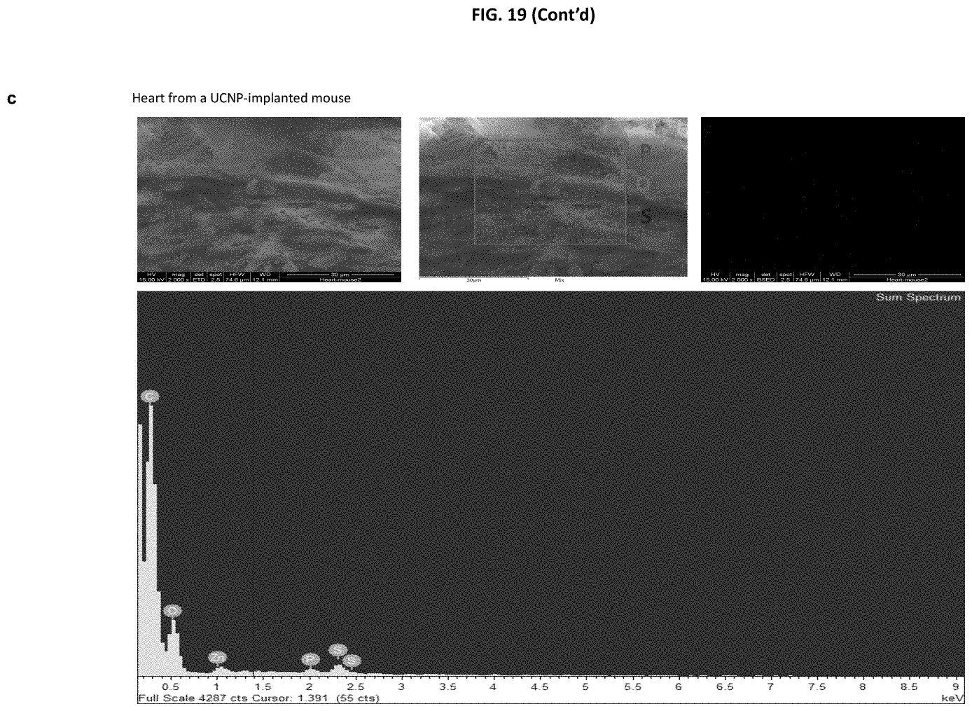

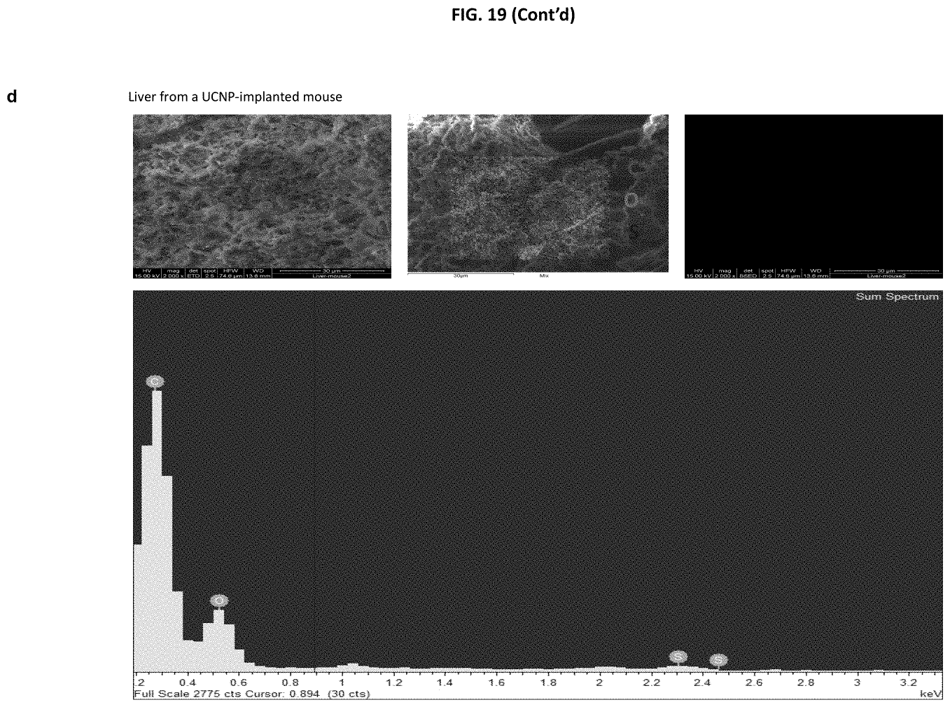

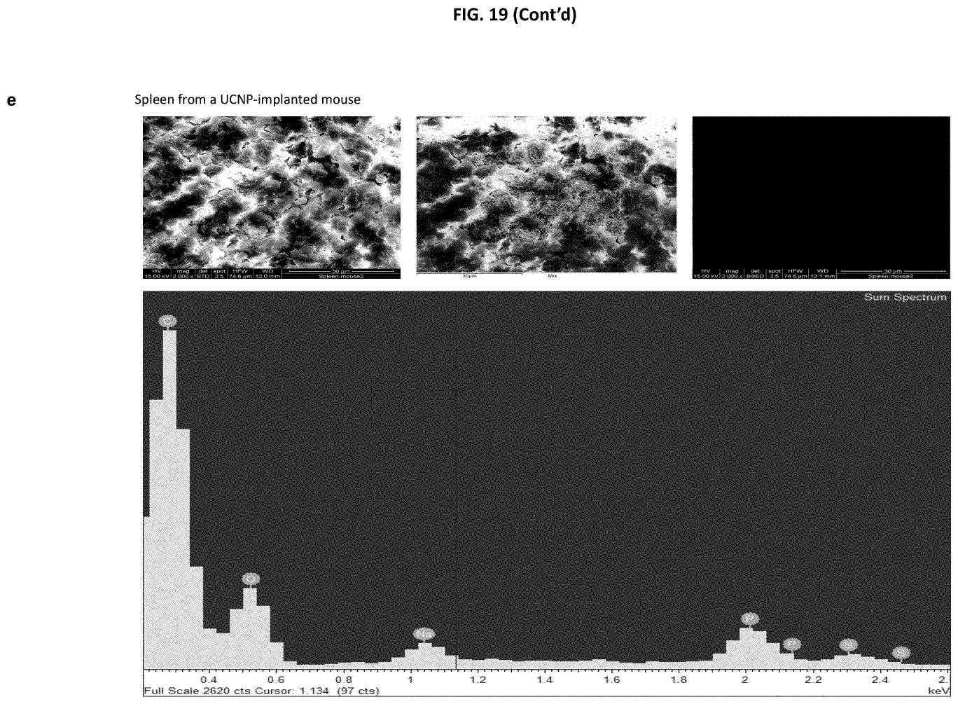

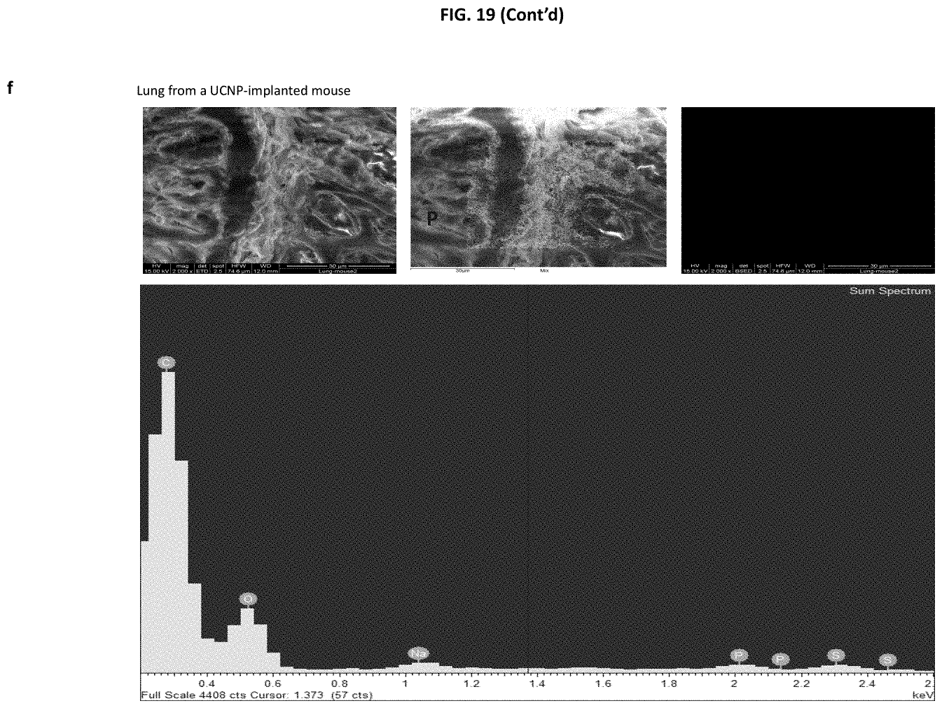

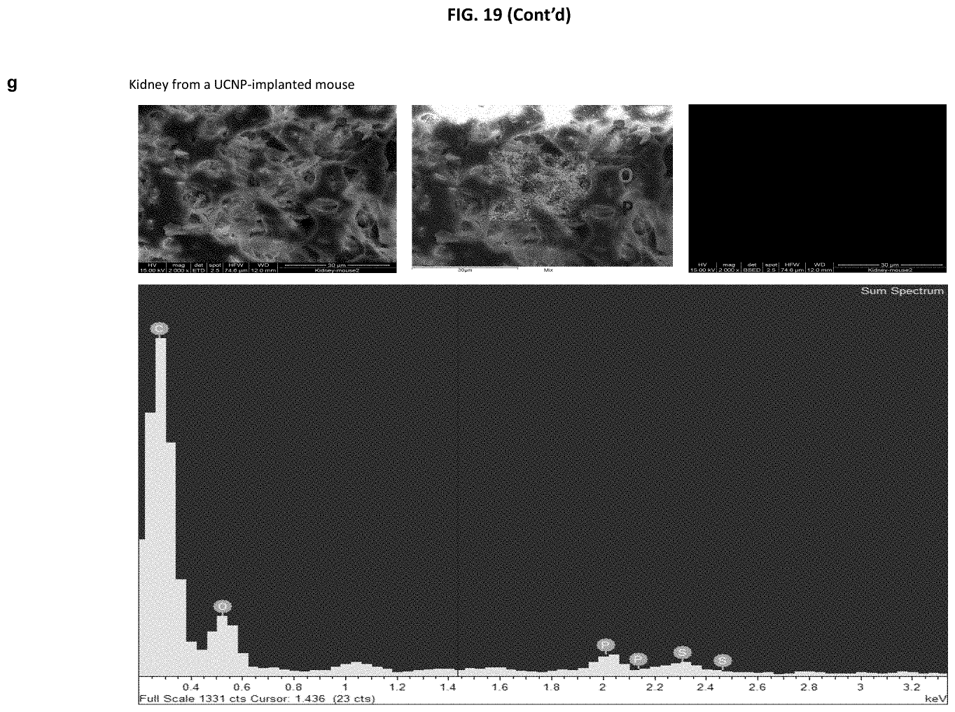

Next, in order to assess the ability of anti-CD19 LiCAR-transduced T cells to eliminate antigen-specific melanoma masses (on-target effect), we conducted in vivo experiments with intradermal melanoma models, in which tumor cells bearing the non-cognate antigen (B16-OVA) and cognate antigen (B16-OVA-hCD19) were implanted into each flank of the same C57BL/6J mice (FIG. 3f-h). After 9 days of tumor establishment, the tumor sites were injected with a mixture composed of engineered WT CAR (the positive control) or LiCAR CD8⁺ T cells (2×10⁶) and 150 μg of UCNPs, in the presence or the absence of pulsed MR light stimulation for 8-9 subsequent days (980 nm at a power density of 250 mW/cm²; 2 h/day; in the pulses of 20 sec ON+5 min OFF). As the positive control, WT CAR T-cells exhibited tumor antigen-specificity by killing tumors made of B16-OVA-hCD19 cells, but they did not kill B16-OVA cells that lack hCD19 (FIG. 14a). As the stringent negative control, the MR light-treated groups injected with UCNPs alone without LiCAR T-cells did not seem to affect the overall tumor growth (FIG. 15b-c). These results ruled out the possible complications from UCNPs and MR light per se. For the LiCAR T-cell treated group, MR light stimulation was found to substantially suppress B16-OVA-hCD19 tumor growth in the left flank, while CD19-negative B16-OVA tumors on the right flank remained largely unaffected (FIG. 3g and FIG. 16a). It is noteworthy that the injected UCNPs appeared to be well confined at the injection site or within the tumor mass and thus could be surgically removed after the treatment via upconverting emission imaging guidance (FIG. 17a). Locally injected UCNPs remained structurally intact (FIG. 16a) and did not seem to spread to other major organs, including the tumor-adjacent tissue, heart, liver, spleen, lungs, and kidney (FIG. 17). Furthermore, these UCNPs were found to be stable throughout the time window of the treatment. We monitored UCNPs through TEM at different time points (day 1, day 7, and day 14) and found that the UCNPs were clearly observable with no obvious morphological changes through 28 days (FIG. 18a; Note: longer-term in vivo study was performed in intramuscular UCNP-injected healthy mice). In addition, under 980 nm laser excitation, the UCNP emissions were clearly observable (FIG. 18b). In contrast, no appreciable UCNP emission was detected from other major organs in the same mouse. Hence, the UCNPs used in our study remained stable in vivo, both physically and optically, for at least 28 days. These findings also indicate that there is no observable nanoparticle leakage from the injected site in tumor to other organs. To more rigorously rule out the potential trace leakage of the nanoparticle to the surrounding tissue and major organs, we performed a systematic element analysis of the nanoparticle distribution in living tissues by using an energy-dispersive X-ray spectroscopy (EDS) coupled SEM system. The lanthanide elements (Y and Yb) as well as Si of the silica shell were only detectable in the UCNP-injected tumor (FIG. 19a). In all the other tissues/organs, including the tumor surrounding tissues (FIG. 19b), heart, liver, spleen, lung, and kidney (FIG. 19c-g), we did not detect lanthanide elements or Si derived from the injected UCNPs. The backscattered electron (BSE) images, which could differentiate heavy atoms (i.e., lanthanide elements) from light atoms, also agreed with the element mapping results: only the UCNP-injected tumor (FIG. 19a, right), but not other surrounding tissue or major organs (FIG. 19b-g, right), was clearly visible due to the presence of Ln³⁺. As summarized in Table S2, the composition of elements in the UCNP-injected tumor largely agreed with the composition of silica-coated NaYbF₄:Tm@NaYF4 UCNPs. By contrast, other tissues were mainly composed of the physiologically-relevant elements, such as O, C, S, and P. Together, these results firmly established that UCNPs tend to be accumulated in the tumor after injection and could be removed by imaging guided surgery without appreciable leakage to the surrounding tissues and other major organs. Nonetheless, the removal of UNCPs is optional given its excellent biocompatibility in living tissues.

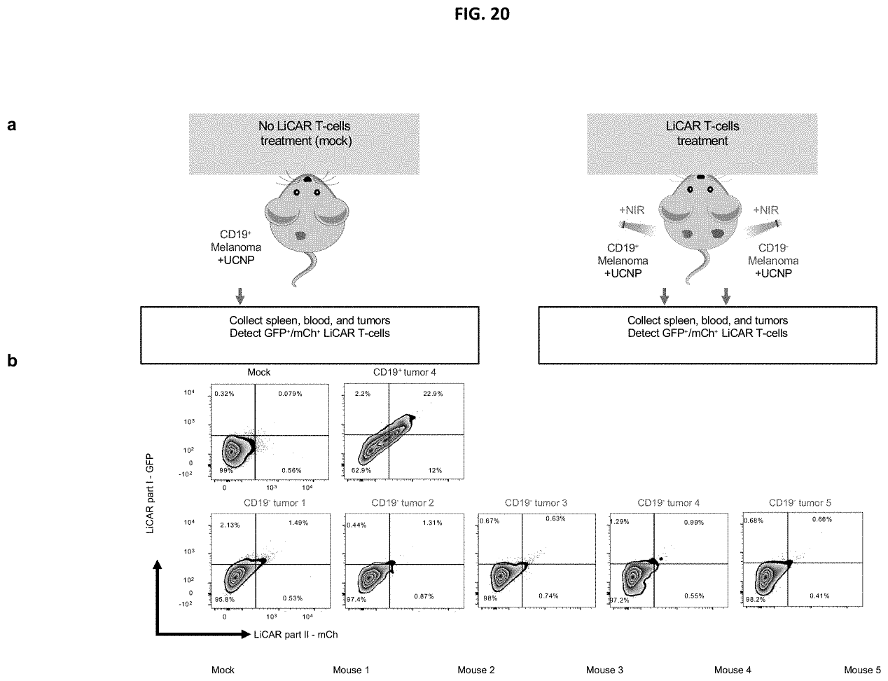

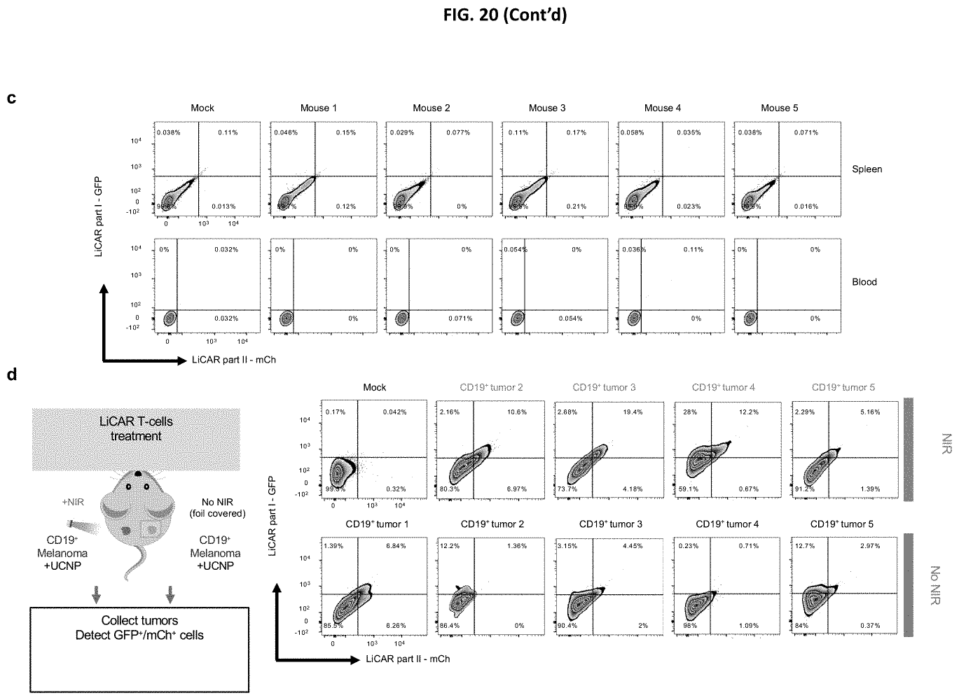

Because the engineered LiCAR T-cells expressed both GFP- and mCherry-tagged components, we further analyzed the population of surviving and/or locally expanded LiCAR T-cells within tumor masses 9 days after injection by flow cytometry (FIG. 20). As anticipated, a significant portion of dual-colored LiCAR T-cells was detected within the CD19⁺ tumors, but not in those formed by CD19-negative B16-OVA cells (FIG. 20a-b). In addition, we did not observe a significant presence of LiCAR T-cells in the peripheral lymphoid organs (such as the spleen) or in the peripheral blood at the endpoint (FIG. 20c). There are two possible explanations for this finding: First, the locally-injected, MR light-activated LiCAR T-cells could not efficiently migrate to distal tissues. Alternatively, in the absence of NIR light and cognate tumor antigens, the extra-tumorous LiCAR T cells were not activated and may be eliminated after 9 days after injection. Regardless, limited extra-tumoral distribution of LiCAR T-cells could be beneficial for the spatial control of therapeutic activity. Taken together, we have established a robust nano-optogenetic platform to temporally control LiCAR T-cells in order to achieve tumor-specific immune responses in vivo.

To demonstrate the spatiotemporal control of LiCAR T-cells to kill tumors in vivo, we implanted B16-OVA-hCD19 cells into each flank of C57BL/6J mice. After tumor formation at day 9, both tumor sites were injected with a mixture of CD8³⁰ LiCAR T-cells and UCNPs, followed by exposure to pulsed MR light on the left side or shielded from light with aluminum foil on the right side (FIG. 3h and FIG. 16b). We found that tumor regression or clearance was only observed in the MR light-treated side, but not in the site shielded from light (FIG. 3h and FIG. 16b). Within the tumor masses, we detected a higher amount of viable LiCAR T-cells, as reflected by a stronger intensity of the GFP/mCherry double-positive population at day 9 following MR light stimulation (FIG. 20d). Together, our data have firmly established the feasibility of spatiotemporal control over engineered T cell activation and selective tumor killing only at the desired tumor sites.

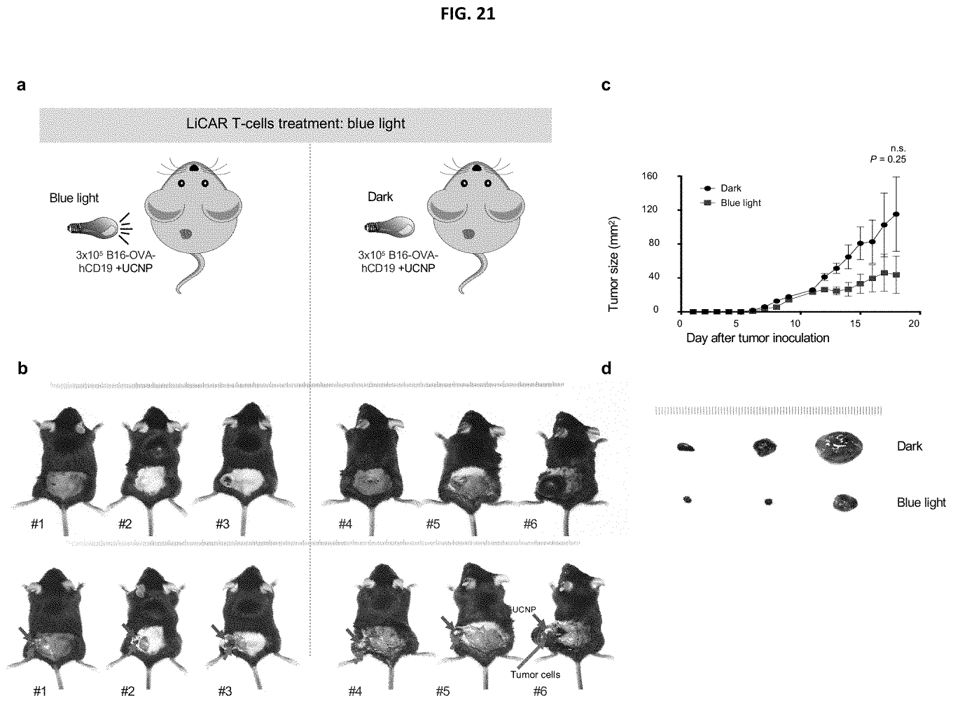

In parallel, we performed similar experiments under blue light illumination. We did not detect a statistically significant difference between the dark and lit groups (FIG. 21). This finding is consistent with the tacit notion that blue light can penetrate no more than 1 mm in living tissues, thereby failing to effectively activate LiCAR T-cells within the tumor sites. (Barolet, et al. 2008 Semin Cutan Med Surg 27, 227-238.) However, this bottleneck has been overcome by taking our nano-optogenetic immunomodulation approach using the injectable and removable enhanced MR-to-blue emission UCNP method.

LiCAR T-Cells Mitigate Side-effects Associated with Immunotherapy

We have demonstrated that our LiCAR system can be controlled with high spatiotemporal precision in targeting tumors both in vitro and in vivo. Therefore, we anticipated that the LiCAR system could mitigate the side-effects caused by constitutively activated CAR T-cells, as most prominently known as “on-target, off-tumor”cytotoxicity and cytokine release syndrome (CRS).

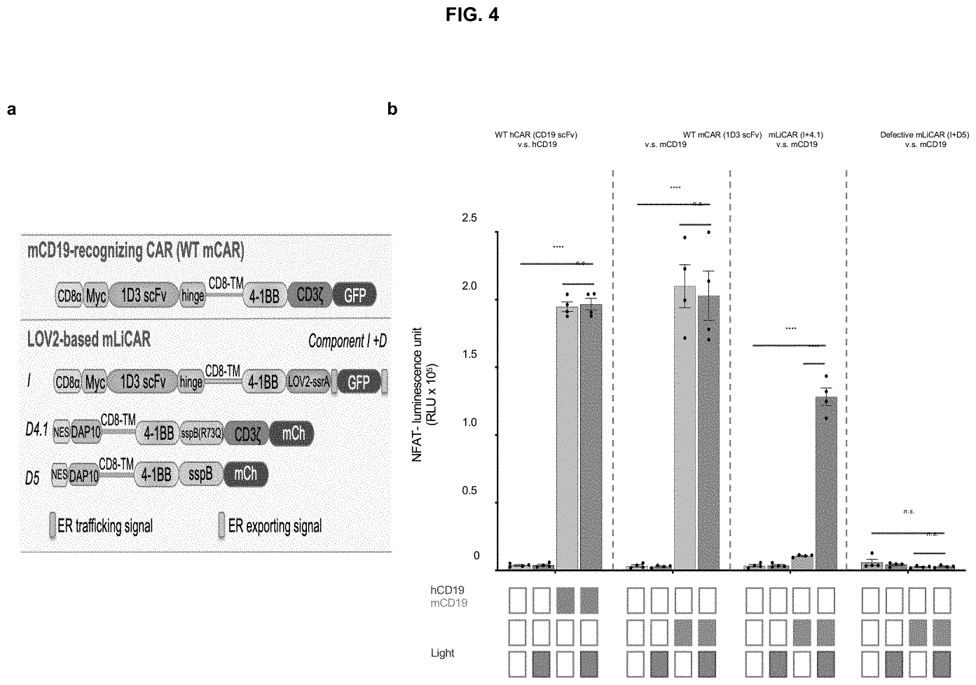

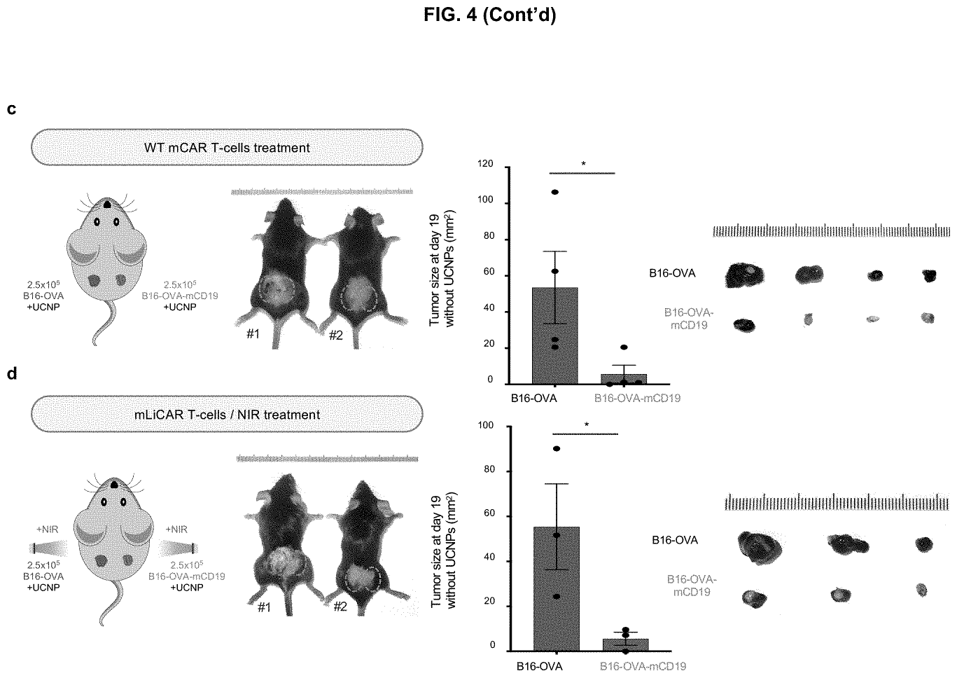

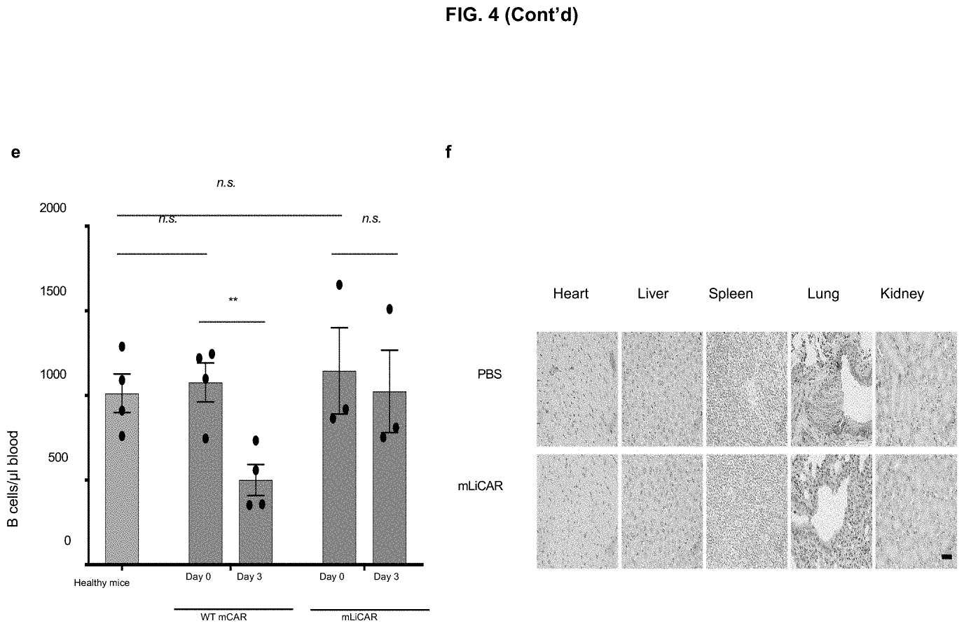

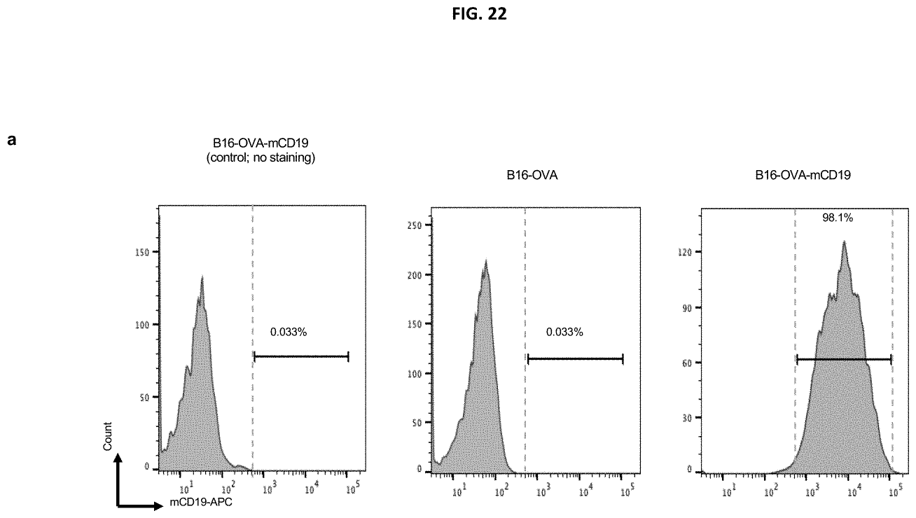

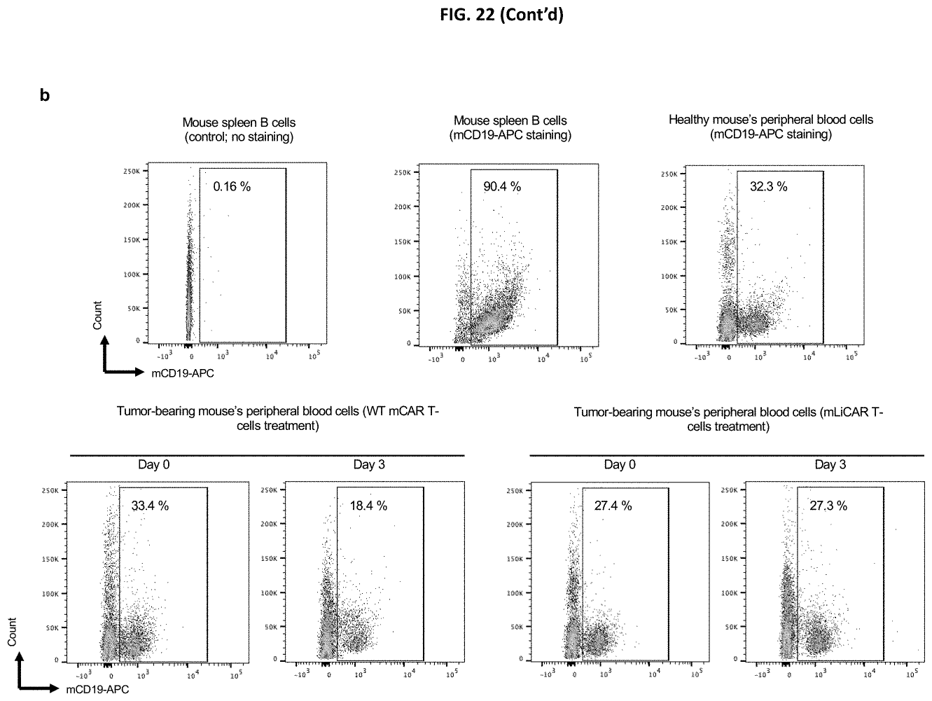

To more rigorously evaluate the “on-target, off-tumor” side effects of LiCAR compared to the conventional CAR in a syngeneic mouse model of tumor, we designed a new set of mouse-specific LiCARs (mLiCARs; FIG. 4a), in which the hCD19-recognizing scFv was replaced by a mouse version derived from a mouse anti-CD19 mAb (1D3 ScFv)³⁹. In order to examine whether 1D3 ScFv can recognize the mouse-specific CD19 (mCD19) antigen as well as to assess the degree of T cell activation, we again used the NFAT-Luc reporter assay (FIG. 4b). NFAT-Luc Jurkat T cells expressing engineered receptors (WT mCAR or mLiCAR) were engaged with B16-OVA melanoma cells or mCD19-positive B16-OVA-mCD19 cells (FIG. 22a). WT mCAR T-cells only required the presence of mCD19 to be activated and the resultant bioluminescence level was comparable to that triggered by human CD19-targeting WT CAR T-cells (engaged by B16-OVA-hCD19 cells), suggesting that mCAR constructs can efficiently recognize mCD19Ag (FIG. 9b). In the dual presence of the cognate tumor antigen from B16-OVA-mCD19 cells and light stimulation, we detected an increase in bioluminescence, indicating the antigen/light-dependent activation of engineered mLiCAR T cells (combination I+D4.1; FIG. 9b). Again, defective mLiCAR T-cells lacking CD3-ITAMs did not show detectable antigen/light-dependent changes (I+D5; FIG. 4b).

Next, similar to the human LiCAR system, we assessed the ability of anti-mCD19 WT CAR (mCAR) or LiCAR-transduced (mLiCAR) T cells to kill tumor cells in vivo using the same melanoma model, in which tumor cells bearing the non-cognate antigen (B16-OVA) or the cognate antigen (B16-OVA-mCD19) were inoculated into each flank of the same C57BL/6J mouse (FIG. 4c-d). After 9 days of tumor establishment, the tumor sites were injected with either WT mCAR T-cells/UCNPs (FIG. 4c) or mLiCAR CD8⁺ T cells/UCNPs (FIG. 4d). Pulsed MR light stimulation for 10 days (980 nm at a power density of 250 mW/cm²; 2 h/day; pulses of 20 sec ON+5 min OFF) was applied to the mLiCAR group only. Both WT mCAR and mLiCAR T-cells exhibited tumor antigen-specificity by suppressing the growth of tumors composed of B16-OVA-mCD19 cells, but not the B16-OVA cells (FIG. 4c-d), thus validating the on-target effects of mLiCAR T-cells.

B cell aplasia (a low number or absence of B cells) is one of the most frequent life-threatening side effects associated with CD19 CAR T-cell therapy due to the nonspecific expression of CD19 on both B cell malignant clones and nonpathogenic B cells. This “on-target/off-tumor” side-effect results in destruction of B cells and hence low numbers of B-cells in the blood, which often requires a combination treatment with empiric immunoglobulin replacement. (Kansagra, et al. 2019 Biol Blood Marrow Transplant 25, e76-e85.) In order to evaluate the degree of B cell aplasia, we compared the number of B cells in the peripheral blood obtained from the WT mCAR or mLiCAR-treated groups (as shown in FIG. 4c-d) at the day prior to T cell implantation (day 0) and 3 days thereafter (FIG. 4e). The results showed that mWT CAR T-cell treatment triggered B cell aplasia after 3 days by reducing both the B cell number (FIG. 4e) and the B cell percentage in the peripheral blood (FIG. 22b), while this phenomenon was not observed in the mLiCAR group. These findings suggest that the UCNP/photo-controllable LiCAR system could attenuate undesired “on-target, off-tumor” toxicity. Additionally, we also collected the major organs including the heart, liver, and kidney, and performed H&E staining to evaluate pathohistological changes. The result here suggested that mLiCAR T-cells did not attack these major organs during our experimental window (FIG. 4f).

Another prevalent adverse effect following CAR T-cell treatment is the initiation of a cytokine storm associated with uncontrolled immune responses, known as CRS, which is characterized by severe clinical syndromes, including fever, hypotension, neurological changes, multi-organ failure, potentially leading to death. (Dai, et al. 2016 J Natl Cancer Inst 108.) A dramatic elevation of IL-6 is one of the critical hallmarks of CRS, along with elevated granulocyte macrophage colony-stimulating factor (GMCSF), IFN-γ and IL-10. (Lee, et al. 2015 Lancet 385, 517-528; Maude, et al. 2014 N Engl. Med 371, 1507-1517.)

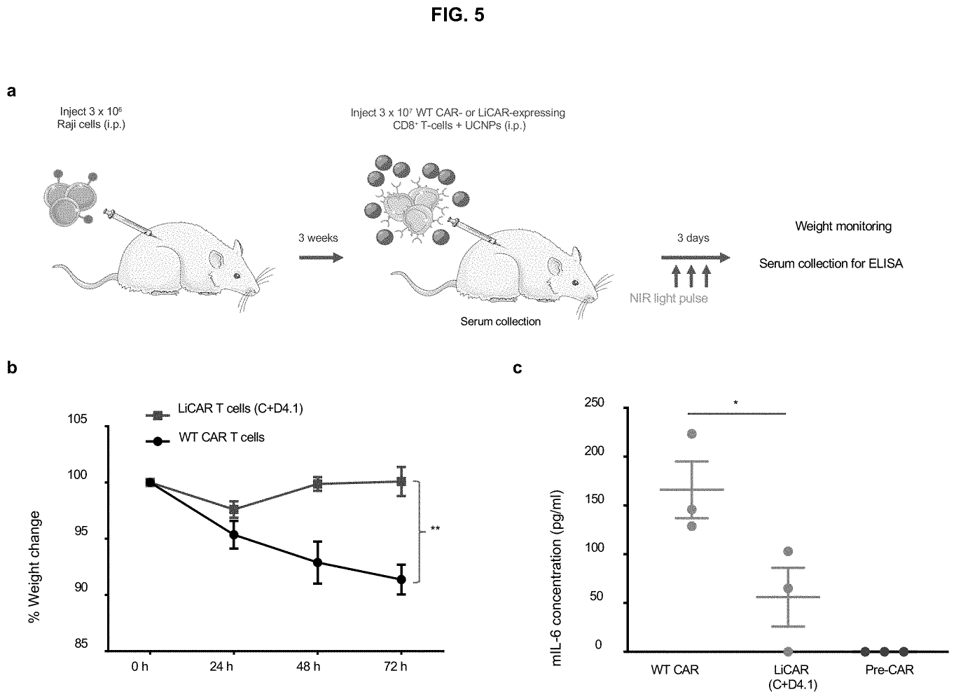

To evaluate the extent of CRS in our system, we adapted a well-established xenograft model of CAR T-cell-induced CRS in SCID-Beige mice recently developed by Giavridis et al., which has been shown to recapitulate major CRS hallmarks seen in the clinic. (Giavridis, et al. 2018 Nat Med 24, 731-738.) We injected (i.p.) large numbers of Raji tumor cells (3×10⁶) and allowed tumor growth for 3 weeks. Thereafter, a large amount of WT CAR-T cells or LiCAR T/UCNPs cells (3×10⁷ cells) were implanted into the tumor sites to elicit CRS, with acute weight loss and IL-6 production as two independent readouts (FIG. 5a). LiCAR-treated mice were subjected to pulsed MR light stimulation for 3 days (980 nm at a power density of 250 mW/cm²; pulse of 20 sec ON, 5 minutes OFF; 2 h/day). In addition to monitoring the weights for 3 consecutive days, sera were collected on day 0 and day 3 and subjected to serum cytokine analysis (mIL-6). The results showed that after 3 days, the mice treated with WT CAR T cells experienced significant weight lost (FIG. 5b). Furthermore, the level of mIL6 was higher in the tumor-bearing mice injected with WT CAR T-cells compared to those injected with LiCAR T/UCNPs under pulsed MR light (FIG. 5c), suggesting that the LiCAR system indeed mitigated the cytokine release syndrome.

### Experimental

### Molecular Cloning and Plasmid Construction

To construct plasmids encoding Component I (A0, C0, E0, and G0), gBlocks gene fragments were synthesized by Integrated DNA Technologies (Coralville, Lowa, USA) to generate the following cDNA sequences: CD8 signal peptide, Myc tag, anti-CD19 scFv, CD8-alpha transmembrane domain, hinge region, and 4-1BB. The synthetic block was inserted between the Nhel-Xhol restriction sites into the mCerulean N1 vector (#27795; Addgene, Watertown, Mass., USA). The optical dimerization components, including CIBN (#60553; Addgene), LOV2-ssrA (#60413; Addgene), CRY2PHR (#89877; Addgene), sspB (#60410; Addgene) were first amplified via standard PCR with KOD Hot Start DNA polymerase (EMD Millipore, Burlington, Mass., USA), and then inserted downstream of 4-1BB between the Xhol-Xmal sites. Two linkers, GSGSGEF and GSGSGSGS, were introduced before and after the dimerization modules, respectively. EGFP was amplified from the hRIP3-eGFP vector (#41387; Addgene) and then inserted between the Agel-Notl restriction sites to replace mCerulean in the backbone vector. For Component I constructs, we generated versions with or without eGFP. To facilitate PM localization, Component I was further modified by inserting (i) a short ER export sequence to the N-terminus and (ii) an ER trafficking signal between the Agel and MluI sites in front of eGFP in the constructs A0 and C0 (FIG. 6a).

To construct plasmids encoding Component II (constructs B1.0/D1.0), we sequentially introduced the 4-1BB, sspB and human CD3ζ intracellular chain derived from gBlocks gene fragments and inserted them between Kpnl, EcoRI, HindIII, and BamHI restriction sites upstream of mCherry in the pmCherry2-N1 vector (#54517; Addgene). CRY2PHR (#89877; Addgene) was used to replace sspB to generate CRY2-based constructs. To create B2.0/D2.0 constructs, we rearranged the intracellular CAR components, and optical dimerization molecules with mCherry, 4-1BB, CD3ζ, and sspB/CRY2 were sequentially inserted into BamHI, XhoI, EcoRI, and Apal sites of pcDNA3.1 (+). The plasma membrane (PM)-tethered Component II constructs (B3/D3) were designed by fusing CD8-alpha hinge and transmembrane domain upstream of the 4-1BB module in the reorganized CAR constructs (4-1BB, CD3ζ, mCherry, and CRY2/sspB). B4/D4 were generated by placing DAP10 (gBlock gene fragment) and the CD8-alpha transmembrane domain upstream of 4-1BB in the B1.0/D1.0 constructs between the XhoI and Kpnl restriction sites. The defective LiCAR constructs (B5/D5) were generated from B4/D4 by removing the T cell-activating CD3 component. To reduce undesired nuclear accumulation, an NES sequence was inserted to the N-terminus of Component II constructs between the NheI and Xhol sites. Constructs D4.1 and D4.2 were generated by replacing sspB from construct D4 and D2 by sspB (R73Q) (#60420; Addgene) between the EcoRI/HindIII and EcoRI/ApaI sites, respectively. Constructs D4.3 and D4.4 were generated by introducing the point mutation A58V into sspB in the parental constructs D4 and D2 by using the QuikChange Lightning Multi Site-Directed Mutagenesis Kit (Agilent Technologies, Santa Clara, Calif., USA) following the manufacturer's instructions.

For viral transduction, the pMSGV1 retroviral vector (#107227; Addgene) and MIGR1 (#27490; Addgene) were used as the backbones into which all the optimized CAR or LiCAR components were inserted between two modified restriction enzyme sites, HpaI and PacI. The helper plasmids including the gag/pol viral packaging vector (#14887; Addgene), a modified vector encoding the amphotropic envelope glycoprotein RD114 (#17576; Addgene), and packaging vector pcl-Eco (#12371; Addgene) were acquired from Addgene.

### Mammalian Cell Culture, Transfection and Fluorescence Microscopy

An NFAT-dependent luciferase reporter (NFAT-Luc) Jurkat cell line was used to examine NFAT-dependent gene transcription as previously described. (He, et al. 2015 Near-infrared photoactivatable control of Ca(2+) signaling and optogenetic immunomodulation. Elife 4.) Human cancer cell lines (K562 myelogenous leukemia cells (#CCL-243), Daudi (#CCL-213) and Raji cell lymphoblasts (#CCL-86)) were purchased from the American Type Culture Collection (ATCC, Manassas, Va., USA), and cultured in Roswell Park Memorial Institute (RPMI 1640) medium with L-glutamine (#MT10040CV, Thermo Fisher Scientific, Waltham, Mass., USA) supplemented with 10% FBS, 100 units/ml penicillin and 100 μg/mL streptomycin (Gibco, Big Cabin, Okla., USA). B16-OVA and the B16-OVA mouse melanoma cell lines transduced with an amphotropic virus containing human CD19 (hCD19) (gifts from Dr. Anjana Rao, La Jolla Institute for Immunology) were maintained in Dulbecco's Modified Eagle Medium (DMEM) (#MT10013CV, Thermo Fisher Scientific) supplemented with 10% FBS, 100 units/ml penicillin and 100 μg/mL streptomycin (Gibco). The CD19 level of each cell line was quantified by staining the cell surface with a monoclonal antibody against CD19 (APC-conjugated; #17-0199-42; eBioscience, San Diego, Calif., USA; FIG. 8a) in FACS buffer (PBS, 2% BSA, and 2 mM EDTA) at 4° C. for 30 min. The stained cells were washed three times with FACS buffer, and the levels of fluorescence protein were determined by using the LSRII flow cytometer (BD Biosciences, San Jose, Calif., USA). Cells were sampled at a medium flow rate, and at least 10,000 cells were counted for each condition. FACSDiva8.0 (BD Biosciences) and FlowJo software v10.5.3 (TreeStar, Ashland, Oreg., USA) were used to analyze the data (APC³⁰ cell populations).

HeLa cells from ATCC (#CCL-2) were maintained in Dulbecco's Modified Eagle Medium (DMEM) (#MT10013CV, Thermo Fisher Scientific) supplemented with 10% FBS, 100 units/ml penicillin and 100 μg/mL streptomycin (Gibco) at 37° C. in a humidified atmosphere under 5% CO₂. For confocal imaging, 10⁵ cultured cells were seeded on 35 mm glass-bottom dishes. After 24 h, LiCAR components I (1,000 ng) and II (600 ng) were co-transfected into HeLa cells using Lipofectamine 3000 (Life Technologies; Carlsbad, Calif., USA) according to the manufacturer's instructions. Confocal imaging was performed at 24 h post-transfection by using an inverted Nikon Eclipse Ti-E microscope customized with Nikon A1R+ confocal laser sources (405/488/561/640 nm). A 488-nm laser was used to excite GFP, which also served as an internal light source for photo-stimulation in some cases, and a 561-nm laser was used to excite mCherry fluorescence. The cells were subjected to two dark-light cycles, in which each cycle had fifteen repetitions of 1-s stimulation and either 3-min of image acquisition for LOV2-based constructs or 6 minutes for CRY2-based constructs. At least four GFP- or mCherry-positive cells were selected to calculate the cytosolic fluorescence at selected areas before and after photo-stimulation (F/F₀).

### Isolation and Culture of Primary Human T Cells

Peripheral blood mononuclear cells (PBMCs) were collected from blood samples of healthy blood donors through the Gulf Coast Regional Blood Center (Houston, Tex., USA) by density gradient centrifugation using the Ficoll-Paque Plus media (#GE17-1440-02; Sigma, St. Louis, Mo., USA). PBMCs were washed three times with sterile PBS and resuspended at a concentration of 1×10⁶ cells/ml in PBS supplemented with 2% BSA and 2 mM EDTA. CD8⁺ T cells from PBMCs were enriched by magnetic cell sorting, using negative selection kits (#130-096-495; Miltenyi Biotec, Auburn, California, USA). The purity of the CD8⁺ cell populations was determined using flow cytometry by staining with APC-conjugated anti-human CD8 (#344721; Biolegend). CD8⁺ T cells were cultured in RPMI L-glutamine medium supplemented with 10% FBS, 1X NEAA (non-essential amino acid medium; #1939940; Gibco), 1 mM sodium pyruvate (#13-115E; Lonza, Houston, Tex., USA), 10 mM HEPES (#15630-080; Gibco), 0.55 μM 2-mercaptoethanol (#21985023; Gibco), 100 units/ml penicillin, and 100 μg/mL streptomycin. Recombinant human IL-2 (#PHC0027; Gibco) was used at a final concentration of 100 IU/mL for culturing CD8⁺ cells.

### Isolation and Culture of Mouse T Cells

Spleens were excised from 6-12 week-old C57BL/6 mice and put in 5 ml of FACS buffer. Each spleen was then transferred to the 100 p.m cell strainer atop a 35 mm plate containing 2 ml of FACS buffer. The plunger of a small syringe was used to crush the spleen in the strainer. The cell suspension from the plate beneath was collected and briefly centrifuged. Red blood cells were removed from the cell pellets by resuspension in 1 ml of ACK lysis buffer (#10-548E; Lonza) for 1 minute. After that, AKC buffer was diluted by adding 9 ml FACS buffer. The cells were then harvested, washed one more time, and resuspended at a concentration of 1×10⁶ cells/ml in FACS buffer. Mouse spleen CD8⁺ T cells were enriched by magnetic cell sorting using negative selection kits (#130-104-075; Miltenyi Biotec). The purity of the CD8⁺ cell populations was determined using flow cytometry by staining with anti-mouse CD8a eFluor 450 (#48-0081-82; eBioscience). Mouse CD8⁺ T cells were cultured in TexMACS media (#130-097-196; Miltenyi Biotec) supplemented with 10% FBS, 100 units/ml penicillin, and 100 μg/mL streptomycin. 400 μg/mL anti-mouse CD3 (#50-139-2707, Fisher Scientific), 400 μg/mL anti-mouse CD28 (#50-562-020, Fisher Scientific), and 100 IU/mL recombinant mouse IL-2 (#PMC0025; Gibco) was freshly added to the medium freshly each time used.

### Viral Transduction of Jurkat T Cells and Primary T Cells

Retroviruses encoding the conventional CAR or engineered CARs were packaged in HEK293T cells (#CRL-3216; ATCC) transfected with the corresponding retroviral vector pMSGV1, the gag/pol viral packaging vectors (#14887; Addgene) and a modified vector encoding the amphotropic envelope glycoprotein RD114 (#17576; Addgene) using the iMFectin DNA Transfection Reagent (#17200-101; Gendepot, Katy, Tex., USA). For mouse T cell transduction, the retroviruses were packed in Plate-E cells (#RV-101; Cell Biolabs, San Diego, Calif., USA) transfected with the packaging vector pcl-Eco (#12371; Addgene) and MIGR1-WT CAR, MIGR1-LiCAR, or MIGR1-defective CAR constructs. The supernatant containing packaged viruses was collected twice at 48 hours and 72 hours after transfection.

Cultured Jurkat T cells at a concentration of 10⁵ cells/ml were co-incubated with 2 ml of the 0.45 μM filtered virus supernatant and 10 μg/ml of polybrene per well in a 12-well plate format. Plates containing cultured cells were centrifuged at 2000×g for 2 hours at 32° C. and incubated 2 more hours at 37° C. The viral supernatant was removed and replaced with fresh RPMI medium supplemented with 10% FBS, 100 units/ml penicillin and 100 μg/mL streptomycin. Cells were repeatedly transduced four times to yield a higher transduction efficiency.

For human primary T cells, the virus-containing supernatants were first concentrated using the Amicon Ultra-15 Centrifugal Filter (#UFC903024; Millipore Sigma, St. Louis, Missouri, USA). Before performing T cell transduction, human primary CD8⁺ T cells were activated for 48 hours using the Dynabeads Human T-Activator CD3/CD28 system (#11132D; Thermo Fisher Scientific) at a bead-to-cell ratio of 1:1. Retroviral transduction of primary T cells was performed by mixing concentrated viruses and RetroNectin (#T100B; Takara Bio, Mountain View, Calif., USA) with T cells according to the manufacturer's instructions.

For mouse primary T cells, CD8⁺ T cells were activated overnight in IgG (H+L) goat anti-Hamster (#PI31115, Fisher Scientific)-coated plates and soluble anti-mouse CD3 (#50-139-2707, Fisher Scientific) and anti-mouse CD28 (#50-562-020, Fisher Scientific) in mouse T cell culture medium. Mouse T cells were transduced with 2 ml of 0.45-μM filtered virus supernatant and 10 _{K}g/ml of polybrene per well in a 12-well plate. Both human and mouse primary T cells were repeatedly transduced by centrifugation two times to achieve efficient transduction. Expression of transgenes was confirmed by detecting fluorescent reporters using an LSRII flow cytometer (BD Biosciences).

### In Vitro Quantification of NFAT-Luciferase (NFAT-Luc) Reporter Activity of Jurkat-Luc T Cells

Jurkat T cells expressing conventional CARs, LiCAR, or defective LiCAR (10⁵ cells/well) were co-cultured with cognate CD19-positive Raji cells or non-cognate CD19-negative K562 cells at indicated effector T cell: target cell (E/T) ratios or at the ratio of 1:3 in a 96-well flat-bottom microplate (#E17073EF; Greiner Bio-one, Monroe, N.C., USA). Plates with cells were incubated at 37° C. in a humidified atmosphere under 5% CO₂ and either kept in the dark or subjected to photostimulation (470 nm at a power density of 40 mW/cm²) for 1 to 25 minutes and then with pulsed blue light (10-30 sec ON, 100 sec OFF) for up to 8 hours. Cell pellets were then harvested, and luciferase activity was assayed by using the Dual Luciferase Reporter Assay System (Promega, Fitchburg, Wis., USA) on the Cytation 5 luminescence microplate reader (BioTek, Winooski, Vt., USA). Data plots were generated by using the Prism version 8.0.0 software (GraphPad, San Diego, Calif., USA).

### ELISA Measurements of Cytokine Production

Jurkat cells expressing conventional CARs, LiCAR, or defective LiCAR (10⁵ cells/well) were mixed with either CD19⁺ Raji cells or CD19⁻K562 cells at an E/T ratio of 1:3, while mouse CD8 T cells transduced with viruses encoding CAR constructs were co-cultured with B16-OVA or B16-OVA-hCD19 at the indicated E:T ratios. The cells were incubated in the dark or exposed to blue light (470 nm at a power density of 40 mW/cm²) continuously for 1 to 25 minutes and then to pulsed blue light (10-30 sec ON, 100 sec OFF) for up to 12 hours. Cell supernatants were collected and analyzed with BD OptEIA Human IL-2 ELISA Set (#555190; BD Biosciences, San Jose, Calif., USA) or mouse IFN-γ ELISA set (#88-7314-22, Invitrogen, Carlsbad, Calif., USA) according to the manufacturer's instructions. Briefly, one day before collecting cell supernatant, a 96-well flat-bottom microplate (#E17073HT; Greiner Bio-one) was coated with human anti-IL-2 or mouse anti-IFN-γ antibody (at a dilution of 1:1,000 in PBS) at 4° C. overnight. On day 2, the plate was washed with 200 μl PBS/0.05% Tween 20 and then blocked with 1% BSA/PBS for 2 h at room temperature. Cell supernatants were diluted 1:10 times in 1% BSA/PBS before being added into the plate wells. A series of cytokine standard dilutions were applied to obtain a standard curve. The plate was then incubated at 4° C. overnight. The next day, 100 μl of biotin-conjugated detection antibody (1:1,000 in 1% BSA/PBS) was added and incubated with cell supernatants for 1 hour at RT. The plate was then washed and incubated with HRP streptavidin (1:3000 in 1% BSA/PBS) at RT for 30 min. After a final wash, each well was incubated with 100 μl of the tetramethyl-benzidine substrate solution (#34028, Thermo Fisher Scientific). 50 μl of 2.5N H₂SO4 (#35348, Honeywell Fluka, Mexico City, Mexico, USA) was added to each well to stop the reaction. For mIL-6 quantification, mouse blood sera were collected and analyzed with an IL-6 Mouse ELISA Kit (#KMC0062, Invitrogen, Carlsbad, Calif., USA) according to the manufacturer's instructions. The absorbance of each well was measured at 450 nm using the Cytation 5 luminescence microplate reader (BioTek). The concentration of samples was calculated based on the standard curve, and data were replotted with the Prism software (GraphPad).

### Flow Cytometry Analysis on CD69 Surface Expression in T Cells

Jurkat T cells transduced with conventional CARs, LiCAR, or defective LiCAR (10⁵ cells/well) were co-cultured with either CD19-negative (K562) or CD19-positive (Daudi/Raji) target tumor cells at a ratio of 1:3 in 96-well flat-bottom microplates (#E17073EF; Greiner Bio-one). The light stimulation was given for 20 min at a power density of 40 mW/cm² and then with pulsed blue light (10 sec ON, 60 sec OFF) for 10 h. After incubation, cells were washed and stained with an Alexa Fluor 700 conjugated anti-human CD69 antibody (#310922; Biolegend) at 4° C. for 30 min in FACS buffer. Cells were washed three times in PBS and then analyzed by a BD LSRII cytometer (BD Biosciences). Data were analyzed using the FACSDiva8.0 (BD Biosciences) and FlowJo software v10.5.3 (TreeStar).

### Tumor Cell Killing Assays

Human CD8⁺ T cells expressing conventional CARs, LiCAR, or defective LiCAR and the cognate CD19⁺ Daudi target cells used in this assay were maintained with a viability of over 97%. The effector T cells were co-cultured with target cells at an E/T ratio ranging from 1:1 to 1:3 using a CD8 T cell medium in 96-well plates. Cells were evenly distributed into two plates, with one plate shielded from light as the control in the dark state and the other subjected to photostimulation (470 nm, power density of 40 mW/cm²) for 20 min every 2 hours for the first 8 h, followed by pulsed blue light (10 s ON, 60 s OFF) for 16 h.

The mixture of human CD8⁺ T and Daudi cells was then harvested and stained with the SYTOX Blue dye (#S11348; Invitrogen) at a final concentration of 100 nM for 15 min at 4° C. in FACS buffer. The cells were then washed twice and resuspended in FACS buffer and subjected to flow cytometry analysis by using a BD LSRII cytometer or a BD FACSAria sorter (BD Biosciences). The FlowJo software v10.5.3 (TreeStar) was used to calculate the death rate of targeted B cells (gated on GFP⁻, mCh⁻, and SYTOX⁺).

For time-lapse fluorescence microscopy, engineered CD8⁺ T-cells were immobilized on 35-mm glass-bottom dishes by using 0.1 mg/ml poly-L-lysine (#2840311; EMD Millipore). The CD19⁺ Daudi cells and SYTOX Blue (100 nM) were subsequently added to the well in T cell culture media. Fluorescence images were acquired at 37° C. and 5% CO₂ in a humidified atmosphere at 40x magnification. The time-lapse recording lasted for about 5 hours at an interval of 2 min under blue light illumination (blue LED at 470 nm with a power density of 40 mW/cm²). The tumor cell killing activity of LiCAR T-cells (GFP⁺, mCh⁺) was monitored by their engagement with Daudi cells to induce target cell death, which was made visible by the SYTOX Blue nucleic acid staining dye.

For ex vivo murine melanoma cells killing assay, B16-OVA-hCD19 cells were seeded onto 96 well glass bottom plates (#655892; Greiner Bio-one) with 10³ cells per well. Cells were incubated overnight for attachment. Mouse CD8⁺ T cells expressing CAR constructs were added to pre-seeded B16-OVA-hCD19 cells at the indicated E/T ratios. One plate was kept in the dark and another plate was subjected to blue light illumination (470 nm with a power density of 40 mW/cm²) for 20 min every 2 hours for the first 8 h, followed by pulsed blue light (10 s ON, 60 s OFF) for 16 h. The plates were then washed three times with PBS to remove unattached T cells and dead B16-OVA-hCD19 cells. The surviving B16-OVA-hCD19 cells, which remained attached to the plate bottom, were visualized by DAPI staining. A high-content confocal imaging system (In cell Analyzer 6000; GE Healthcare Life Sciences, Chicago, Ill., USA) was used to capture the images of each well. Cell numbers were quantified by using the IN Cell Developer Toolbox version 1.9 (GE Healthcare). Data plots were generated by using the Prism version 8.0.0 software (GraphPad).

Synthesis of β-NaYbF₄:0.5% Tm @NaYF₄ Core-Shell UCNPs

The β-NaYbF₄:0.5% Tm@NaYF₄UCNPs were prepared by a three-step thermolysis method. In the first step, CF₃COONa (0.50 mmol), Yb(CF₃COO)₃ (0.4975 mmol) and Tm(CF₃COO)₃ (0.0025 mmol) precursors were mixed with oleic acid (5 mmol), oleyamine (5 mmol), and 1-octadecene (10 mmol) in a two-neck round bottom flask. The mixture was heated to 110° C. to form a transparent solution followed by 10 min of degassing. Then the mixture was heated to 300° C. at a rate of 15° C./min under dry argon flow, and maintained at 300° C. for 30 min to form the α-NaYbF4:0.5% Tm intermediate UCNPs. After the mixture cooled down to room temperature, the α-NaYbF₄:0.5% Tm intermediate UCNPs were collected by centrifugal washing with excessive ethanol (7500 g, 30 min). In the second step, the α-NaYbF4:0.5% Tm intermediate UCNPs were redispersed into oleic acid (10 mmol) and 1-octadecene (10 mmol) together with CF₃COONa (0.5 mmol) in a new two-neck round bottom flask. After degassing at 110° C. for 10 min, this flask was heated to 325° C. at a rate of 15° C/min under dry argon flow and maintained at 325° C. for 30 min to complete the phase transfer from α to β. After the mixture cooled to room temperature, the β-NaYbF₄:0.5% Tm core UCNPs were collected by precipitation with an equal volume of ethanol followed by centrifugation (7500×g, 30 min). The β-NaYbF₄:0.5% Tm core UCNPs were stored in hexane (10 mL). In the third step, the as-synthesized β-NaYbF₄:0.5% Tm core UCNPs served as cores for the epitaxial growth of core-shell UCNPs. Typically, a hexane stock solution of β-NaYbF₄:0.5% Tm core UCNPs was transferred into a two-neck round bottom flask, and the hexane was sequentially evaporated by heating. CF₃COONa (0.25 mmol) and Y(CF₃COO)₃ (0.25 mmol) were introduced as UCNP shell precursors with oleic acid (10 mmol) and 1-octadecene (10 mmol). After 10 min of degassing at 110° C., the flask was heated to 325° C. at a rate of 15° C./min under dry argon flow, and maintained at 325° C. for 30 min to complete the shell crystal growth. After the mixture cooled to room temperature, the β-NaYbF₄:0.5% Tm@NaYF₄ core-shell UCNPs were collected by precipitation with an equal volume of ethanol followed by centrifugation (7500×g, 30 min). β-NaYbF₄:0.5% Tm@NaYF₄ core-shell UCNPs were stored in hexane (10 mL). The control sample of β-NaYF₄:30%Yb,0.5% Tm@NaYF₄ core-shell UCNPs were synthesized similarly, except for changing the amount of Ln(CF₃COO)₃ according to the stoichiometric ratio.

### Synthesis of Silica Coated Core-Shell UCNPs

The silica shell was coated onto the core-shell UCNPs via a modified Stober method. In a typical process, 4 mL of the core-shell UCNP hexane solution was added to 21 mL hexane in a 50 mL one-neck round bottom flask. 1.5 mL of Igepal CO-520 was added to the solution which was kept in a water bath while sonicated for 2 minutes. 160 μL of ammonia was added to the solution. After 30 minutes of stirring, add 80 μL of TEOS (Tetraethoxysilane) was added to the solution. After 2 days of stirring, the silica-coated core-shell UCNPs were collected by precipitated with an equal volume of ethanol and centrifugation afterward (7500×g, 30 min) for 3 times. This silica-coated core-shell UCNPs were stored in water (20 mL).

### Cell Proliferation Assay

Cell proliferation was quantified by using the WST-1 colorimetric assay (#05015944001; Sigma) according to the manufacturer's instructions. Briefly, on day 1, B16-OVA and B16-OVA-hCD19 cells were seeded in a 96-well plate (10³ cells per well) in 200 μl of DMEM medium supplemented with 10% FBS, 100 unit/ml penicillin and 100 μg/mL streptomycin. After incubation for the indicated times, 20 μl of WST-1 was added to each well and incubated at 37° C., 5% CO₂ for 2 h. The cell plates were shaken for 1 min on a shaker. The absorbance of each well against a background control (medium with WST-1 only) was measured at 450 nm using a Cytation 5 luminescence microplate reader (BioTek). Data plots were generated by using the Prism version 8.0.0 software (GraphPad).

### Mouse Syngeneic Models of Melanoma

All animal studies were approved by the Institutional Animal Care and Use Committee of Texas A&M University Institute of Biosciences and Technology. On day 0, 6-12-week-old C57BL/6J mice (either sex) were inoculated intradermally with 2.5-5×10⁵ B16-OVA and B16-OVA-hCD19 or B16-OVA-mCD19 cells depending on each experiment. When the tumors became visible, their sizes were measured with a digital caliper every day, and the tumor area was calculated in millimeters (length x width). The tumors were allowed to grow for 8 additional days after inoculation. On day 9, 2×10⁶ mouse CD8 T cells expressing CAR constructs and 150 μg of UCNP were co-injected into each tumor. From day 10, LiCAR-treated mice (hLiCAR or mLiCAR) were subjected to pulsed near-infrared light treatment (980 nm at a power density of 250 mW/cm²; pulse: 20 sec ON, 5 minutes OFF for 2 hours per day). On day 16 to 19, tumors were collected from euthanized mice. For the analysis of hLiCAR T cells residing within the tumors, tumors were collected, perfused in PBS, cut into small pieces and enzymatically digested with 5 ug/ml of Liberase TL (#298569; Roche, Basel, Switzerland) for 1 hour at 37° C. Tumor cells were then filtered by using a 100 μm cell strainer. Cells were washed twice in PBS to remove cell debris and resuspended in a FACS buffer. The number of adoptively transferred hLiCAR T cells was determined by detecting hLiCAR fluorescence protein using the LSRII flow cytometer (BD Biosciences). FACSDiva8.0 (BD Biosciences) and FlowJo software v10.5.3 (TreeStar, Ashland, Oreg., USA) were used to analyze the data (gated on the GFP⁺/mCh⁺ population). For the analysis of hLiCAR T-cells residing within the spleen or blood, spleen cells were isolated by crushing the spleen on the strainer as described above while blood cells were collected from retro-orbital sinus by glass capillary from anesthetized mice. Spleen and blood cells were then treated with ACK lysis buffer (#10-548E; Lonza) to remove red blood cells. The spleen and blood cells were washed twice before performing flow cytometry analyses.

### Mouse B Cell Quantification

WT mCAR T or mLiCAR T-cells/UCNPs were implanted into C57BL/6 mice bearing B16-OVA or B16-OVA-mCD19 tumors. mLiCAR-transferred mice were subjected to pulsed near-infrared light treatment (980 nm at a power density of 250 mW/cm²; pulse: 20 sec ON, 5 minutes OFF for 2 hours per day). On day 0 and day 3, 200 μl of blood was collected from the retro-orbital sinus by glass capillary or from tail-clip from anesthetized mice. RBC was then removed by using ACK lysis buffer (#10-548E; Lonza). Total cell counts in peripheral blood were determined by using a TC20 Automated Cell Counter (Biorad, CA, USA). The percentage of B cells in the total cell population was quantified using the LSRII flow cytometer (BD Biosciences) after staining cells with a monoclonal antibody against mCD19 (APC-conjugated; #17-0193-82; eBioscience) in FACS buffer at 4° C. for 30 min. The stained cells were washed three times with FACS buffer and were sampled at a medium flow rate with 10,000 cells counted. FACSDiva8.0 (BD Biosciences) and FlowJo software v10.5.3 (TreeStar, Ashland, Oreg., USA) were used to analyze the data (APC⁺ cell populations). Based on the percentage of B cells in the cell population, we calculate the amount of B cells per μl blood.

Xenogeneic models using SCID-Beige mice to assess CRS

We used a recently well-adopted murine model using SCID-Beige mice to evaluate CRS in vivo². Briefly, on day 0, 3×10⁶ Raji cells were intraperitoneally injected into 6- to 8-week-old female C.B-Igh-1b/GbmsTac-Prkdc^{scid}-Lyst^{bg} N7 mice (Taconic Biosciences, New York, USA). After 3 weeks, mice were divided into three groups: one group was injected intraperitoneally with 3×10⁷ WT CAR-expressing CD8 T-cells, while another group was injected with 3×10⁷ LiCAR-expressing CD8 T cells. The control group was treated with PBS only. 3 days after injection, LiCAR-transferred mice were subjected to pulsed near-infrared light treatment (980 nm at a power density of 250 mW/cm²; pulse: 20 sec ON, 5 minutes OFF for 2 hours per day). All the mice were weighed every day to evaluate the weight change. On day 0 and day 3, blood cells were collected from the retro-orbital sinus by glass capillary or by tail-clip from anesthetized mice. Blood was left to clot for 30 min at room temperature and then centrifuged 6000×g at 4° C. for 10 min. Serum was collected and kept frozen at −80° C. until analysis. mIL-6 in the serum was detected using ELISA (#KMC0061, Invitrogen) following the manufacturer's instructions.

### Element Analysis of the UCNP Distribution in Mouse Tissues

Element mapping was performed in the Electron Microscopy Facility at the University of Massachusetts Medical School. Dissected mouse tissues were fixed by immersion in 4% paraformaldehyde for 2 days at 4° C. and then kept frozen. After thawing the tissues, a second fixation was performed with 2.5% glutaraldehyde in 0.1.M Na Cacodylate buffer (ph 7.2) for 30 min at room temperature. The fixed samples were then washed three times in the same fixation buffer. Following the third wash, the samples were dehydrated through a graded series of ethanol (10, 30, 50, 70, 85, 95% for 20 min each) to three changes of 100% ethanol, and then they underwent critical point drying in liquid CO₂. The dried mouse tissues were then cut to expose the inside, and they were mounted onto aluminum stubs with carbon tape making sure that the exposed surfaces were facing up. All the samples were then carbon coated with 3 nm of carbon to ensure conductivity. The specimens were examined using an FEI Quanta 200 FEG MK II scanning electron microscope at 15 Kv accelerating voltage under two modalities, secondary electron and backscattered imaging. The samples were also examined using an EDS system (Oxford Link Inca 350 x-ray spectrometer) to determine the element distributions in the tissues. The element spectrum, element mapping, and atomic ratio of each element were presented as the raw data obtained in the INCA EDS system without further manipulation.

### Cytotoxicity Assessment of UCNPs

B16-OVA-hCD19 cells were cultured in Dulbecco's modified Eagle's medium (DMEM) containing 10% fetal bovine serum (FBS), 100 μg/ml streptomycin and 100 U/ml penicillin at 37° C. in a humidified incubator containing 5% CO2 and 95% air. The medium was replenished every other day and the cells were subcultured after reaching confluence. Cell viability was examined by using a well-established MTT assay. In brief, B16-OVA-hCD19 cells were plated in a 96-well plate. After 12 h, the nanoparticles were added at different concentrations (0, 20, 40, 60, 80, 100 μg mL⁻¹). The cells were incubated another 24 h under 5% CO2 at 37° C. MTT solution (5.0 mg mL-1, 50 μL) was added to every well and left for 4 h. The old cell culture medium was removed carefully and 200 μL DMSO was added to every well. A microplate reader (Bio-Rad) was used to record the absorption at 595 nm. Cell viability (%)=OD value test/OD value control×100%.

### In Vivo Assessment of Potential UCNP Toxicity

For histological analysis, on the 1st, 7th and 14th day after injection of UCNPs (1 mg/ml, 150 μL), mice were sacrificed and major organs (heart, liver, spleen, lung, kidney, and tumor) were dissected for H&E staining. Mice injected with 150 μl PBS were used as the control. In parallel, before the mice were euthanatized, blood samples (approximately 0.5 ml) were collected for blood panel analyses and blood chemistry tests.

### Flow Cytometry Analysis of Macrophages

To quantify the population of macrophages within tumor sites or spleens, tumor tissue or spleens were minced into small pieces, treated with 1 mg/ml collagenase I (Gibco) for 1 h at 37° C. and grounded using the rubber end of a 10-ml syringe (BD Biosciences). Cells were filtered through nylon mesh filters (Corning, cell strainer, 70 μm nylon). The single cells were collected by centrifugation (800×g, 5 min), and blood cells in the tumor tissue were eliminated by cold NH₄Cl lysis. The cell suspensions were washed in cold PBS containing 2% FBS. The dispersed cells were stained with fluorescence-labeled antibodies FITC-anti-F4/80 (total macrophages) and APC-anti-CD86 (M1 macrophages) or PE/Cy7-anti-CD206 (M2 macrophages) following the manufacturer's instructions. All antibodies were diluted 200 times. Flow cytometric analyses were performed on an LSRFortessa (BD Biosciences) and analyzed using FlowJo Software (Tree Star).

### In Vivo Evaluation of UCNP Stability

150 μL UCNPs (1 mg/ml) were injected into the tumors in tumor-bearing mice or the leg muscles of healthy mice. On the 1st, 7th, 14th and 28th day after UCNP injection, mice were sacrificed, and the UCNP-injected tumor or muscle was isolated and fixed with 4% paraformaldehyde (PFA)/0.25% glutaraldehyde in 0.1 M sodium phosphate buffer (PB) overnight. After several washes in 0.1 M cacodylate buffer (pH 7.4), sections were postfixed with 1% osmium tetroxide (Sigma) in 0.1 M cacodylate buffer for 1 h. After six washes with water for 1 h each and dehydration through a graded ethanol series of 10%, 30%, 50%, 70%, 85%, 95% and 100% EtOH (10 min each for 10%-70%, 20 min each for 85%, 30 min each for 95% and 100% (three times)). UCNP-injected tumor or muscle was treated with propylene oxide for 10 min twice and then immersed in freshly prepared 50% (v/v) Durcupan resin in propylene oxide overnight for resin infiltration (Sigma). UCNP-injected tumor or muscle was then immersed in freshly prepared Durcupan resin for six times, 1 hour each. Sections were then transferred to freshly prepared Durcupan resin contained in a tube and left in a 60° C. oven for 2 days for resin curing. The UCNP-injected tumor/muscle was excised out from the flat-embedded sections and glued onto a resin block for ultrathin sectioning. 70 nm ultrathin sections were cut with a diamond knife (Diatome), collected in formvar-coated single-slot copper grid and briefly counterstained with 2% uranyl acetate in 50% ethanol and 0.4% lead citrate. Sections were observed under electron microscope (Philips CM10 Electron Microscope) at 100 KeV accelerating voltage. The sample preparation and imaging were performed in the Electron Microscopy Facility at the University of Massachusetts Medical School.

Sequence Information

CAR is derived from:

Signal peptide from T-cell surface glycoprotein CD8
alpha chain
MALPVTALLLPLALLLHAARP
FMC63 human CD19 single chainvariable fragment
DIQMTQTTSSLSASLGDRVTISCRASQDISKYLNWYQQKPDGTVKLLIYHTSRLHSGVPSRFS
GSGSGTDYSLTISNLEQEDIATYFCQQGNTLPYTFGGGTKLEITGSTSGSGKPGSGEGSTKGE
VKLQESGPGLVAPSQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGTSVTVSS
Transmembrance domain from T-cell surface glycoprotein
CD8 alpha chain
TTTPAPRPPTPAPTIASQPLSLRPEACRPAAGGAVHTRGLDFACDIYIWAPLAGTCGVLLLSL
VITLYC
Human costimulatory domain 4-1BB (CD137)
KRGRKKLLYIFKQPFMRPVQTTQEEDGCSCRFPEEEEGGCE
Mouse costimulatory domain 4-1BB (CD137)
VQNSCDNCQPGTFCRKYNPVCKSCPPSTFSSIGGQPNCNICRVCAGYFRFKKFCSSTHNAECE
CIEGFHCLGPQCTRCEKDCRPGQELTKQGCKTCSLGTFNDQNGTGVCRPWTNCSLDGRSVLKT
GTTEKDVVCGPPVVSFSPSTTISVTPEGGPGGHSLQVLTLFLALTSALLLALIFITLLFSVLK
WIRKKFPHIFKQPFKKTTGAAQEEDACSCRCPQEEEGGGGGYEL
Human T-cell-specific surface glycoprotein CD28
NKILVKQSPMLVAYDNAVNLSCKYSYNLFSREFRASLHKGLDSAVEVCVVYGNYSQQLQVYSK
TGFNCDGKLGNESVTFYLQNLYVNQTDIYFCKIEVMYPPPYLDNEKSNGTIIHVKGKHLCPSP
LFPGPSKPFWVLVVVGGVLACYSLLVTVAFIIFWVRSKRSRLLHSDYMNMTPRRPGPTRKHYQ
PYAPPRDFAAYRS
Murine T-cell-specific surface glycoprotein CD28
IEFMYPPPYLDNERSNGTIIHIKEKHLCHTQSSPKLFWALVVVAGVLFCYGLLVTVALCVIWT
NSRRNRGGQSDYMNMTPRRPGLTRKPYQPYAPARDFAAYRP
Human T-cell surface glycoprotein CD3 zeta chain
LRVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Murine T-cell surface glycoprotein CD3 zeta chain
RAKFSRSAETAANLQDPNQLYNELNLGRREEYDVLEKKRARDPEMGGKQQRRRNPQEGVYNAL
QKDKMAEAYSEIGTKGERRRGKGHDGLYQGLSTATKDTYDALHMQTLAPR
DAP 10
MIHLGHILFLLLLPVAAAQTTPGERSSLPAFYPGTSGSCSGCGSLSLP
LOV2-SsrA
LATTLERIEKNFVITDPRLPDNPIIFASDSFLQLTEYSREEILGRNCRFLQGPETDRATVRKI
RDAIDNQTEVTVQLINYTKSGKKFWNVFHLQPMRDYKGDVQYFIGVQLDGTERLHGAAEREAV
CLIKKTAFQIAEAANDENYF
cpLOV2 v1
EKNFVITDPRLPDNPIIFASDSFLQLTEYSREEILGRNCRFLQGPETDRATVRKIRDAIDNQT
EVTVQLINYTKSGKKFWNLFHLQPMRDQKGDVQYFIGVQLDGTEHVRDAAEREGVMLIKKTAE
NIDEAAKELGGGSGGSGGGLATTLERI
cpLOV2 v2
PDNPIIFASDSFLQLTEYSREEILGRNCRFLQGPETDRATVRKIRDAIDNQTEVTVQLINYTK
SGKKFWNLFHLQPMRDQKGDVQYFIGVQLDGTEHVRDAAEREGVMLIKKTAENIDEAAKELGG
GSGGSGGGLATTLERIEKNFVITDPRL
cpLOV2 v3
PETDRATVRKIRDAIDNQTEVTVQLINYTKSGKKFWNLFHLQPMRDQKGDVQYFIGVQLDGTE
HVRDAAEREGVMLIKKTAENIDEAAKELGGGSGGSGGGLATTLERIEKNFVITDPRLPDNPII
FASDSFLQLTEYSREEILGRNCRFLQG
cpLOV2 v4
SGKKFWNLFHLQPMRDQKGDVQYFIGVQLDGTEHVRDAAEREGVMLIKKTAENIDEAAKELGG
GSGGSGGGLATTLERIEKNFVITDPRLPDNPIIFASDSFLQLTEYSREEILGRNCRFLQGPET
DRATVRKIRDAIDNQTEVTVQLINYTK
cpLOV2 v5
GDVQYFIGVQLDGTEHVRDAAEREGVMLIKKTAENIDEAAKELGGGSGGSGGGLATTLERIEK
NFVITDPRLPDNPIIFASDSFLQLTEYSREEILGRNCRFLQGPETDRATVRKIRDAIDNQTEV
TVQLINYTKSGKKFWNLFHLQPMRDQK
cpLOV2 v6
TEHVRDAAEREGVMLIKKTAENIDEAAKELGGGSGGSGGGLATTLERIEKNFVITDPRLPDNP
IIFASDSFLQLTEYSREEILGRNCRFLQGPETDRATVRKIRDAIDNQTEVTVQLINYTKSGKK
FWNLFHLQPMRDQKGDVQYFIGVQLDG
SspB
SSPKRPKLLREYYDWLVDNSFTPYLVVDATYLGVNVPVEYVKDGQIVLNLSASATGNLQLTND
FIQFNARFKGVSRELYIPMGAALAIYARENGDGVMFEPEEIYDELNIG
CIBN
MNGAIGGDLLLNFPDMSVLERQRAHLKYLNPTFDSPLAGFFADSSMITGGEMDSYLSTAGLNL
PMMYGETTVEGDSRLSISPETTLGTGNFKAAKFDTETKDCNEAAKKMTMNRDDLVEEGEEEKS
KITEQNNGSTKSIKKMKHKAKKEENNFSNDSSKVTKELEKTDYIHV
SPA1
GSNNTNVDSPRAGKFEHLYRLARGSAFRAGDGDLDSQPRDMDQMLSRIRQQLAGAPSERQNLK
PFMSRRSDQNLEAFSERLRAAGENSIMNAPALISEGVQMKTPVSSSNFSQLLLKRAMKGKGVV
GKNQETPPEFVSDQDLGSKEKKLDISKSPTPHDVLPLKSSPKGNG
CRY2PHR
MKMDKKTIVWFRRDLRIEDNPALAAAAHEGSVFPVFIWCPEEEGQFYPGRASRWWMKQSLAHL
SQSLKALGSDLTLIKTHNTISAILDCIRVTGATKVVFNHLYDPVSLVRDHTVKEKLVERGISV
QSYNGDLLYEPWEIYCEKGKPFTSFNSYWKKCLDMSIESVMLPPPWRLMPITAAAEAIWACSI
EELGLENEAEKPSNALLTRACSPGWSNADKLLNEFIEKQLIDYAKNSKKVVGNSTSLLSPYLH
FGEISVRHVFQCARMKQIIWARDKNSEGEESADLFLRGIGLREYSRYICFNFPFTHEQSLLSH
LRFFPWDADVDKFKAWRQGRTGYPLVDAGMRELWATGWMHNRIRVIVSSFAVKFLLLPWKWGM
KYFWDTLLDADLECDILGWQYISGSIPDGHELDRLDNPALQGAKYDPEGEYIRQWLPELARLP
TEWIHHPWDAPLTVLKASGVELGTNYAKPIVDIDTARELLAKAISRTREAQIMIGAA
BIC1 (BLUE-LIGHT INHIBITOR OF CRYPTOCHROMES 1)
MMNIDDTTSPMAHPIGPSQPPSDQTKQDPPSLPQEAASSVSADKKDLALLEEKPKQSQEEDRV
DTGRERLKKHRREIAGRVWIPEIWGQEELLKDWIDCSTFDTCLVPAGISSARTALVEEARRAA
SASGGLHNRCLILR
ER trafficking signal from inward rectifier potassium
channel 2
KSRITSEGEYIPLDQIDINV
ER exporting signal from inward rectifier potassium
channel 2
FCYENEVGSFCYENEV
Nuclear exporting signal
MNELALKLAGLDLGGSDPPVAT

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An isolated nucleic acid sequence or isolated nucleic acid sequences, comprising:
a first nucleic acid sequence encoding at least one extracellular antigen binding domain, a transmembrane domain, at least one first costimulatory domain, and a first part of an optogenetic dimerizer pair (collectively “Component I”); and
a second nucleic acid sequence encoding at least one second costimulatory domain, an intracellular signaling domain, and a second part of the optogenetic dimerizer pair (collectively “Component II”),
wherein when the first part of the optogenetic dimerizer pair and the second part of the optogenetic dimerizer pair form a fusion dimer upon light induction, Component I and Component II together form a chimeric antigen receptor (CAR).

2. The isolated nucleic acid sequence or sequences of claim 1, wherein the first part of the optogenetic dimerizer pair is fused with one of the at least one first costimulatory domain.

3. The isolated nucleic acid sequence or sequences of claim 1, wherein the first part of the optogenetic dimerizer pair is fused with one of the at least one extracellular antigen binding domain.

4. The isolated nucleic acid sequence or sequences of claim 1, wherein the second part of the optogenetic dimerizer pair is fused with one of the at least one second costimulatory domain.

5. The isolated nucleic acid sequence or sequences of claim 1, wherein the second part of the optogenetic dimerizer pair is fused with the intracellular signaling domain.

6. The isolated nucleic acid sequence or sequences of claim 1, wherein the optogenetic dimerizer pair is selected from the group consisting of: CRY2/CIBN pair, LOV2-ssrA/sspB pair or its modified version using circularly permuted LOV2 (cpLOV2), pMag, CRY2/SPA1 pair, and CRY2/BIC1 pair.

7-8. (canceled)

9. The isolated nucleic acid sequence or sequences of claim 1, wherein the optogenetic dimerizer pair fuses upon excitation of a light having a wavelength in the range of about 450 nm to about 500 nm.

10. (canceled)

11. The isolated nucleic acid sequence or sequences of claim 1, wherein the at least one extracellular antigen binding domain binds to an antigen expressed on a target cell.

12. The isolated nucleic acid sequence or sequences of claim 11, wherein the target cell is a tumor cell.

13. The isolated nucleic acid sequence or sequences of claim 11, wherein the tumor cell is a carcinoma.

14-18. (canceled)

19. The isolated nucleic acid sequence or sequences of claim 1, wherein the transmembrane domain comprising a transmembrane domain of a protein selected from the group consisting of the T-cell receptor (TCR) alpha chain, the TCR betachain, the TCR zeta chain, CD28, CD3 epsilon, CD45, CD4, CD5, CD8, CD9, CD16, CD22, CD33, CD37, CD64, CD80, CD86, CD134, CD137, CD154, or any combination thereof.

20. The isolated nucleic acid sequence or sequences of claim 1, wherein the at least one costimulatory domain comprising a functional signaling domain selected from the group consisting of OX40, CD70, CD27, CD28, CDS, ICAM-1, LFA-1 (CD11a/CD18), ICOS (CD278), DAP10, DAP12, 4-1BB (CD137), or any combination thereof.

21. The isolated nucleic acid sequence or sequences of claim 1, wherein the intracellular signaling domain comprising a functional domain selected from the group consisting of a 4-1BB (CD137); CD28, and CD3 zeta signaling domain, or a combination thereof

22. The isolated nucleic acid sequence or sequences of claim 1, wherein the at least one extracellular antigen binding domain is connected to the transmembrane domain by a linker or spacer domain.

23. The isolated nucleic acid sequence or sequences of claim 1, wherein the at least one extracellular antigen binding domain consists of a single extracellular antigen binding domain.

24. The isolated nucleic acid sequence or sequences of claim 1, wherein the at least one extracellular antigen binding domain comprise two or more extracellular antigen binding domains.

25. The isolated nucleic acid sequence or sequences of claim 1, consisting of one isolated nucleic acid sequence comprising the first nucleic acid sequence encoding Component I and the second nucleic acid sequence encoding Component II.

26. The isolated nucleic acid sequence or sequences of any one of claims 1-24, comprising a first isolated nucleic acid sequence comprising the first nucleic acid sequence encoding Component I and a second isolated nucleic acid sequence comprising the second nucleic acid sequence encoding Component II.

27. A vector comprising a nucleic acid sequence or nucleic acid sequences that comprise:
a first nucleic acid sequence encoding at least one extracellular antigen binding domain, a transmembrane domain, at least one first costimulatory domain, and a first part of an optogenetic dimerizer pair (collectively “Component I”); and
a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a chimeric antigen receptor (CAR).

28-49. (canceled)

50. A cell comprising a nucleic acid sequence or sequences, comprising:
a first nucleic acid sequence encoding at least one extracellular antigen binding domain, a transmembrane domain, at least one first costimulatory domain, and a first part of an optogenetic dimerizer pair (collectively “Component I”); and
a second nucleic acid sequence encoding at least one second costimulatory domain, an intracellular signaling domain, and a second part of the optogenetic dimerizer pair (collectively “Component II”), wherein when the first part of the optogenetic dimerizer pair and the second part of the optogenetic dimerizer pair form a fusion dimer upon light induction, Component I and Component II together form a chimeric antigen receptor (CAR).

51-123. (canceled)
